(19) United States
(12) Patent Application Publication
(10) Pub. No.: US 2004/0142950 A1
(43) Pub. Date: Jul. 22, 2004

(54) Amide and ester matrix metalloproteinase inhibitors

(76) Inventors: Amy Mae Bunker, Ann Arbor, MI (US); Joseph Armand Picard, Canton, MI (US)

Correspondence Address: WARNER-LAMBERT COMPANY, 2800 PLYMOUTH RD, ANN ARBOR, MI, 48105, US

(21) Appl. No.: 10/739,261

(22) Filed: Dec. 18, 2003

Related U.S. Application Data
(60) Provisional application No. 60/440,837, filed on Jan. 17, 2003.

(51) Int. Cl. A61K 31/517; C07D 239/72; C 07D 4 3/02

(52) U.S. Cl. 514/266.2; 514/266.3; 544/286; 544/284

(57) ABSTRACT

This invention provides compounds of Formula I

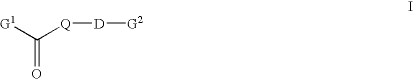

or a pharmaceutically acceptable salt thereof, wherein G¹, Q, D, and G² are as defined above for Formula I. Compounds of Formula I, or a pharmaceutically acceptable salt thereof, are inhibitors of MMP-13. The compounds are useful for treating diseases mediated by MMP-13, including the diseases recited herein such as breast cancer, cartilage damage, rheumatoid arthritis, and osteoarthritis.

### FIELD OF THE INVENTION

This invention relates to a group of amide and ester derivatives that inhibit matrix metalloproteinase enzymes, particularly MMP-13, and are thus useful for treating diseases resulting from tissue breakdown, such as heart disease, including heart failure, multiple sclerosis, arthritis, including osteoarthritis and rheumatoid arthritis, atherosclerosis, including atherosclerotic plaque rupture, age-related macular degeneration, chronic obstructive pulmonary disease, psoriasis, asthma, cardiac insufficiency, inflammation associated with breakdown of extracellular matrix, inflammatory bowel disease, periodontal diseases, and osteoporosis.

### BACKGROUND OF THE INVENTION

Matrix metalloproteinases (sometimes referred to as MMPs) comprise a family of more than twenty naturally occurring enzymes most of which are found in most mammals. Over-expression and activation of MMPs or some other pathological imbalance between MMPs and their naturally occurring inhibitors, namely tissue inhibitors of metalloproteinases (“TIMPs”), has been suggested as factors in the pathogenesis of diseases characterized by the breakdown of extracellular matrix or connective tissues. Pathological imbalance or over-expression and activation of MMP-13 has been directly implicated in diseases such as, for example, osteoarthritis, rheumatoid arthritis, cartilage damage, heart failure, atherosclerotic plaque rupture, inflammation associated with breakdown of extracellular matrix, and breast cancer.

To minimize potential side effects, what is needed to treat patients with MMP-13 mediated diseases is an inhibitor, increasingly preferably a selective, highly selective, or specific inhibitor, of MMP-13. Presently, no selective, highly selective, or specific inhibitor of MMP-13 has been approved by regulatory authorities for treatment of a disease in a mammal. Accordingly, the need continues to find new compounds that are potent and increasingly preferably selective, highly selective, or specific MMP-13 inhibitors. These compounds should also have an acceptable therapeutic index of toxicity versus in vivo efficacy to allow their clinical use for the prevention or treatment of an MMP-13 mediated disease. An object of this invention is to provide a nontoxic group of selective, highly selective, or specific MMP-13 inhibitor compounds characterized as being amides or esters.

### SUMMARY OF THE INVENTION

This invention provides a nontoxic group of amide and ester compounds that are inhibitors, increasingly preferably selective, highly selective, or specific inhibitors, of MMP-13. Particularly, the amide and ester compounds are defined by Formula I below.

1. A compound of Formula I

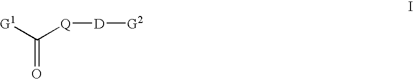

or a pharmaceutically acceptable salt thereof,

wherein:

Each G¹ and G² independently is an unsubstituted or substituted group selected from C₃ to C₇ cycloalkyl-(C₁-C₈ alkylenyl)ₘ-, C₅ or C₆ cycloalkyl-(C₁-C₈ alkylenyl)ₘ-, C₈-C₁₀ bicycloalkyl-(C₁-C₈ alkylenyl)ₘ-, 3- to 7-membered heterocycloalkyl-(C₁-C₈ alkylenyl)ₘ-, 5- or 6-membered heterocycloalkyl-(C₁-C₈ alkylenyl)ₘ-, 8- to 10-membered heterobicycloalkyl-(C₁-C₈ alkylenyl)ₘ-, Phenyl-(C₁-C₈ alkylenyl)ₘ-, Naphthyl-(C₁-C₈ alkylenyl)ₘ-, 5- or 6-membered heteroaryl-(C₁-C₈ alkylenyl)ₘ-, 8- to 10-membered heterobiaryl-(C₁-C₈ alkylenyl)ₘ-, 5- or 6-membered heterocycloalkyl-phenylenyl-(C₁-C₈ alkylenyl)ₘ-, Biphenyl-(C₁-C₈ alkylenyl)ₘ-, 5- or 6-membered heteroaryl-phenylenyl-(C₁-C₈ alkylenyl)ₘ-, 5- or 6-membered heteroaryl-(5- or 6-membered heteroarylenyl)-(C₁-C₈ alkylenyl)ₘ-, Phenyl-L-(phenylenyl)-(C₁-C₈ alkylenyl)ₘ-, Phenyl-L-(5- or 6-membered heteroarylenyl)-(C₁-C₈ alkylenyl)ₘ-, 8- to 10-membered heterobiaryl-phenylenyl-(C₁-C₈ alkylenyl)ₘ-, Phenyl-(5- or 6-membered heteroarylenyl)-(C₁-C₈ alkylenyl)ₘ-, Naphthyl-(5- or 6-membered heteroarylenyl)-(C₁-C₈ alkylenyl)ₘ-, Phenyl-O—(C₁-C₈ alkylenyl)ₘ-, Phenyl-S—(C₁-C₈ alkylenyl)ₘ-, Phenyl-S(O)—(C₁-C₈ alkylenyl)ₘ-, Phenyl-S(O)₂—(C₁-C₈ alkylenyl)ₘ-, Phenyl-(8- to 10-membered heterobiarylenyl)-(C₁-C₈ alkylenyl)ₘ-, and any of the above G¹ and G² groups independently substituted with from 1 to 6 substituents, each independently on a carbon or nitrogen atom, independently selected from C₁-C₆ alkyl, C₁-C₆ alkyl-(G)ₘ-, CN, CF₃, HO, (C₁-C₆ alkyl)-O, (C₁-C₆ alkyl)-S, (C₁-C₆ alkyl)-S(O), (C₁-C₆ alkyl)-S(O)₂, O₂N, H₂N, (C₁-C₆ alkyl)-N(H), (C₁-C₆ alkyl)₂-N, (C₁-C₆ alkyl)-C(O)O-(C₁-C₈ alkylenyl)ₘ-, (C₁-C₆ alkyl)-C(O)O-(1- to 8-membered heteroalkylenyl)ₘ-, (C₁-C₆ alkyl)-C(O)N(H)—(C₁-C₈ alkylenyl)ₘ-, (C₁-C₆ alkyl)-C(O)N(H)-(1- to 8-membered heteroalkylenyl)ₘ-, H₂NS(O)₂—(C₁-C₈ alkylenyl)ₘ-, (C₁-C₆ alkyl)-N(H)S(O)₂—(C₁-C₈ alkylenyl)ₘ-, (C₁-C₆ alkyl)₂-NS(O)₂—(C₁-C₈ alkylenyl)ₘ-, 3- to 6-membered heterocycloalkyl-(G)ₘ-, 5- or 6-membered heteroaryl-(G)ₘ-, (C₁-C₆ alkyl)-S(O)₂—N(H)—C(O)—(C₁-C₈ alkylenyl)ₘ-, and (C₁-C₆ alkyl)-C(O)—N(H)—S(O)₂—(C₁-C₈ alkylenyl)ₘ-;

wherein each substituent on a carbon atom of G¹ or G² may further be independently selected from Halo and HO₂C;

wherein 2 substituents on the same carbon atom of G¹ or G² may be taken together with the carbon atom to which they are both bonded to form the group C═O;

wherein G¹ and G² are not both independently selected from phenyl, benzyl, naphthyl, C₃ to C₇ cycloalkyl-(C₁-C₈ alkenyl)ₘ-, C₈-C₁₀ bicycloalkyl-(C₁-C₈ alkenyl)ₘ-, 3- to 7-membered heterocycloalkyl-(C₁-C₈ alkenyl)ₘ-, and 8- to 10-membered heterobicycloalkyl-(C₁-C₈ alkenyl)ₘ-;

Each m is independently an integer of 0 or 1;

Each G and L is independently selected from CH₂, C(O), N(H), N(C₁-C₆ alkyl), O, S, S(O), and S(O)₂;

Q is O, N(H), or N(C₁-C₆ alkyl);

D is a cyclic diradical group selected from:

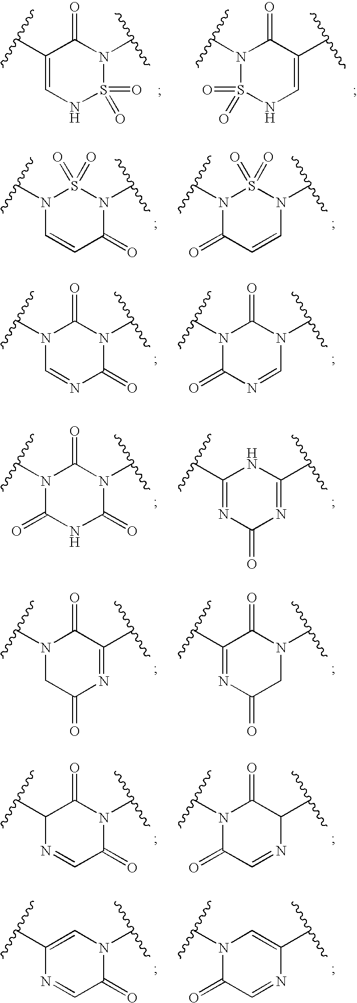

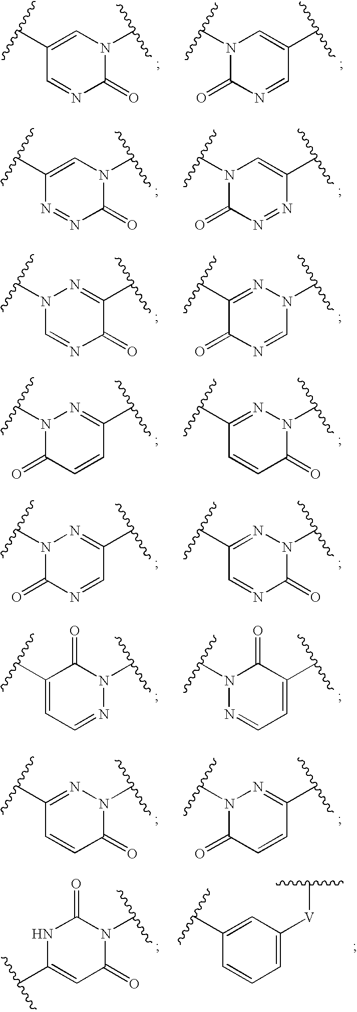

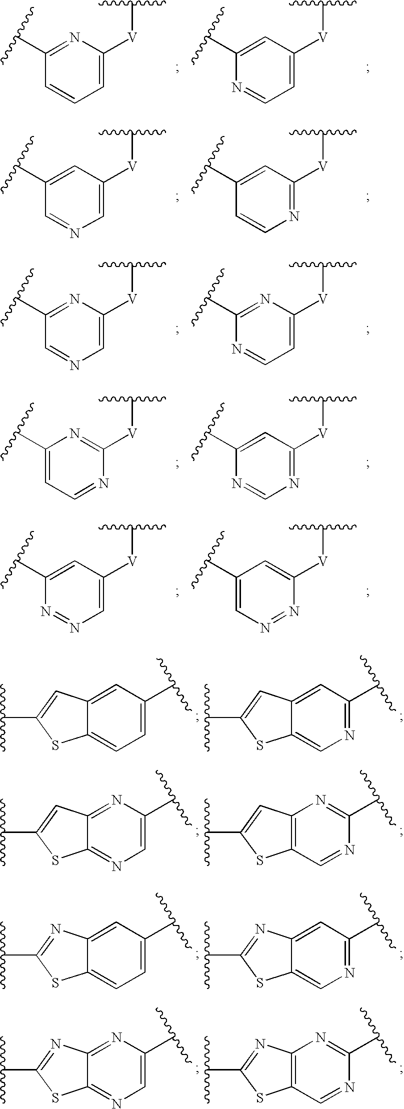

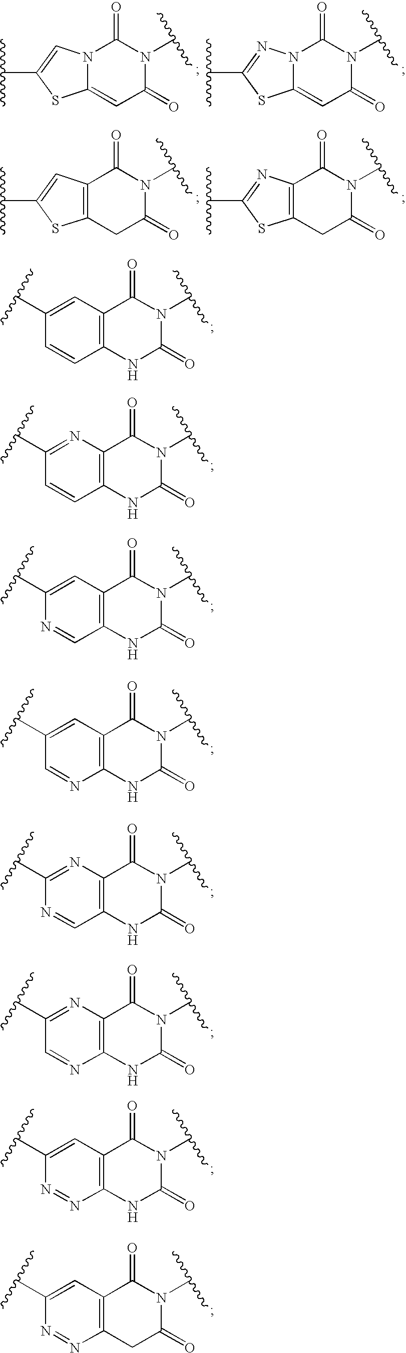

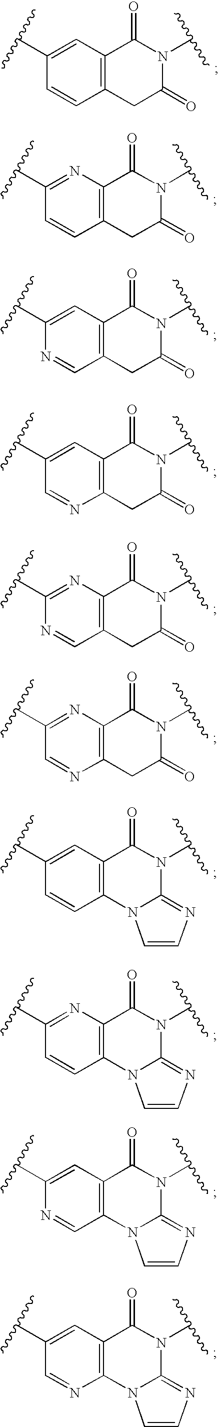

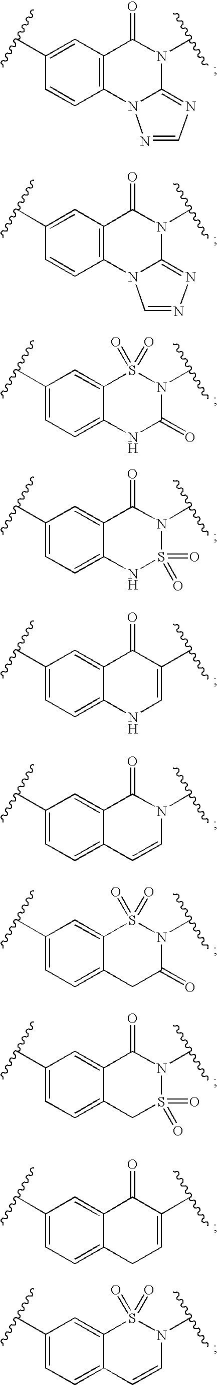

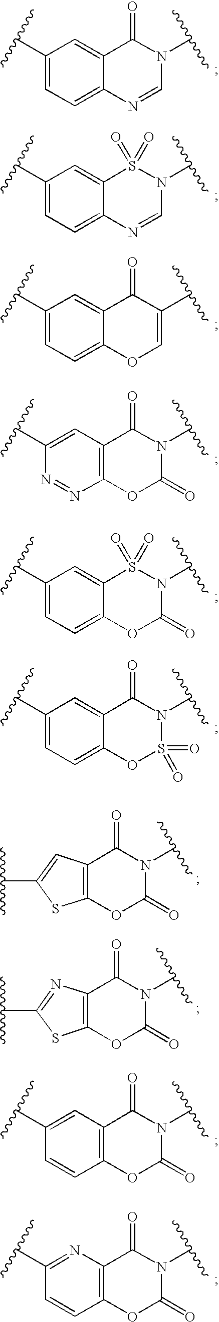

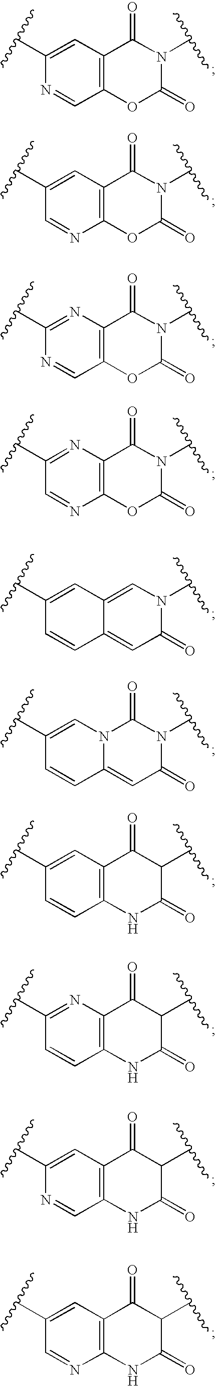

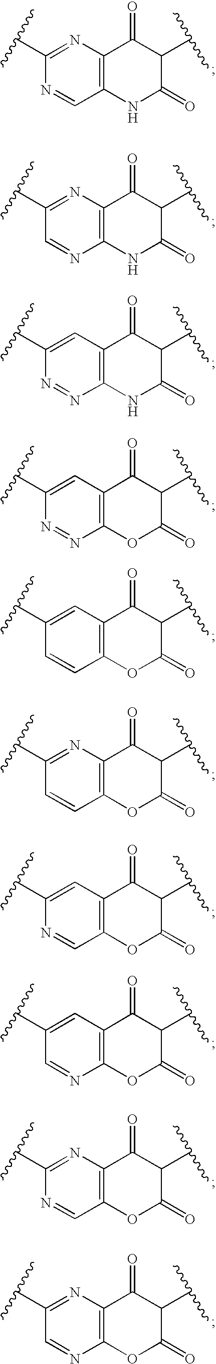

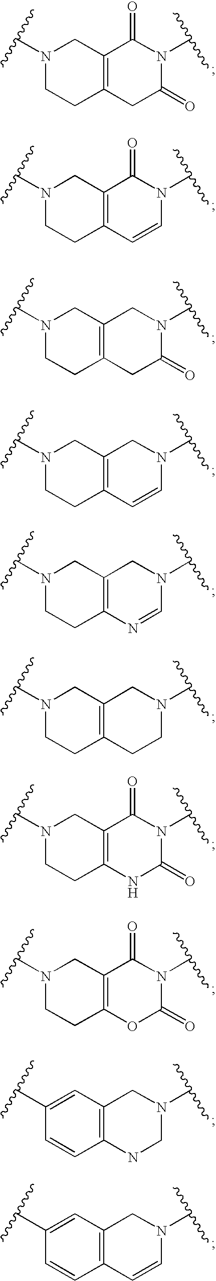

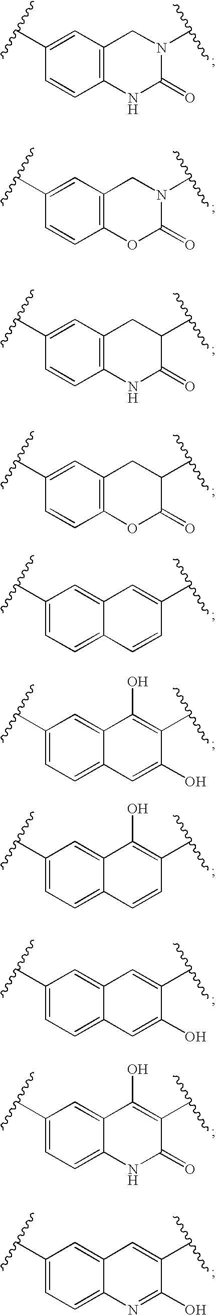

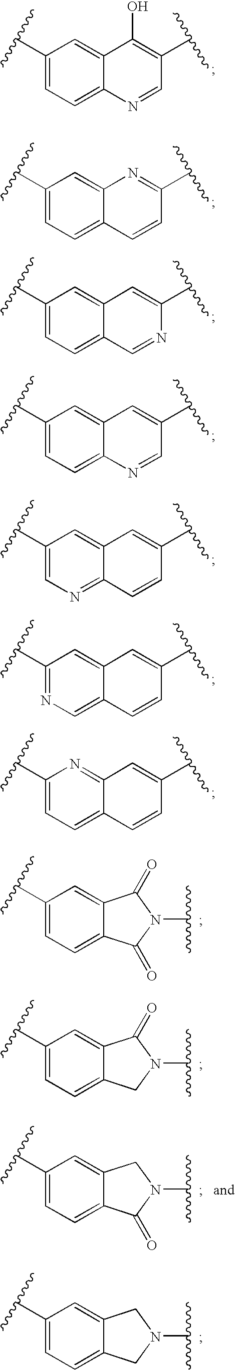

wherein the group D may be unsubstituted or substituted on carbon and nitrogen atoms with 1 or 2 groups independently selected from CH₃, CF₃, N≡C—, CH₃C(O), HO, CH₃O, C(F)H₂O, C(H)F₂O, and CF₃O; and the substituent on a carbon atom in the group D may be further selected from F and Cl; and

V is a 5-membered heteroarylenyl diradical containing carbon atoms and from 1 to 4 heteroatoms selected from 1 O, 1 S, 1 NH, 1 N(C₁-C₆ alkyl), and 4 N, wherein the O and S atoms are not both present, and wherein the heteroarylenyl may optionally be unsubstituted or substituted on a carbon atom or a nitrogen atom with 1 group selected from CH₃, CF₃, N—C—, CH₃C(O), HO, CH₃O, C(F)H₂O, C(H)F₂O, and CF₃O; and the substituent on the carbon atom in the group V may be further selected from F.

2. A further embodiment of this invention is a pharmaceutical composition, comprising a compound of Formula I, or a pharmaceutically acceptable salt thereof, admixed with a pharmaceutically acceptable carrier, excipient, or diluent.

3. Another embodiment of this invention is a method for inhibiting an MMP-13 enzyme in an animal, comprising administering to the animal an MMP-13 inhibiting amount of a compound of Formula I, or a pharmaceutically acceptable salt thereof.

4. A further embodiment is a method for treating a disease mediated by an MMP-13 enzyme, comprising administering to a patient suffering from such a disease an effective amount of a compound of Formula I, or a pharmaceutically acceptable salt thereof.

Further invention embodiments are described as follows:

5. A compound of Formula I, or a pharmaceutically acceptable salt thereof, wherein Q is O.

6. A compound of Formula I, or a pharmaceutically acceptable salt thereof, wherein Q is N(H) or N(C₁-C₆ alkyl).

7. A compound of Formula II

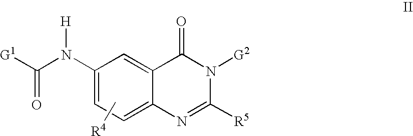

or a pharmaceutically acceptable salt thereof, wherein:

G¹ and G² are as defined above for Formula I; R⁴ is attached at one of the three substitutable benzo carbon atoms of Formula II and R⁴ and R⁵ are each independently selected from H, CH₃, CF₃, N≡C—, CH₃C(O), HO, CH₃O, C(F)H₂O, C(H)F₂O, CF₃O, F, and Cl.

8. A compound of Formula III

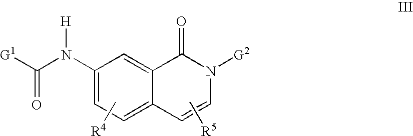

or a pharmaceutically acceptable salt thereof, wherein:

G¹ and G² are as defined above for Formula I; R⁴ is attached at one of the three substitutable benzo carbon atoms of Formula III and R⁵ is attached at one of the two ene carbon atoms of Formula III, and R⁴ and R⁵ are each independently selected from H, CH₃, CF₃, N≡C—, CH₃C(O), HO, CH₃O, C(F)H₂O, C(H)F₂O, CF₃O, F, and Cl.

9. A compound of Formula IV

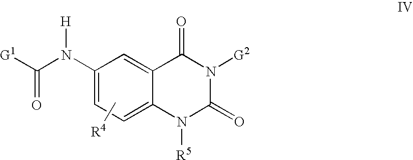

or a pharmaceutically acceptable salt thereof, wherein:

G¹ and G² are as defined above for Formula I; R⁴ is attached at one of the three substitutable benzo carbon atoms of Formula IV and is independently selected from H, CH₃, CF₃, N≡C—, CH₃C(O), HO, CH₃O, C(F)H₂O, C(H)F₂O, CF₃O, F, and Cl; and R⁵ is H, CH₃, CH₃C(O), CN, or CH₃O.

The compound according to any one of Formulas I to IV, or a pharmaceutically acceptable salt thereof, wherein G¹ and G² are as defined in claim 1 except each m is 1 and each C₁-C₈ alkylenyl is independently CH₂ or CH₂ substituted with 1 or 2 substituents independently selected from: C₁-C₆ alkyl-(G)ₘ-; C₁-C₆ alkyl; CN; CF₃; HO; (C₁-C₆ alkyl)-O; (C₁-C₆ alkyl)-S; (C₁-C₆ alkyl)-S(O); (C₁-C₆ alkyl)-S(O)₂; O₂N; H₂N; (C₁-C₆ alkyl)-N(H); (C₁-C₆ alkyl)₂-N; (C₁-C₆ alkyl)-C(O)O-(C₁-C₈ alkylenyl)ₘ-; (C₁-C₆ alkyl)-C(O)O-(1- to 8-membered heteroalkylenyl)ₘ-; (C₁-C₆ alkyl)-C(O)N(H)—(C₁-C₈ alkylenyl)ₘ-; (C₁-C₆ alkyl)-C(O)N(H)-(1- to 8-membered heteroalkylenyl)ₘ-; H₂NS(O)₂—(C₁-C₈ alkylenyl)ₘ-; (C₁-C₆ alkyl)-N(H)S(O)₂—(C₁-C₈ alkylenyl)ₘ-; (C₁-C₆ alkyl)₂-NS(O)₂—(C₁-C₈ alkylenyl)ₘ-; 3- to 6-membered heterocycloalkyl-(G)ₘ-; 5- or 6-membered heteroaryl-(G)ₘ-; (C₁-C₆ alkyl)-S(O)₂—N(H)—C(O)—(C₁-C₈ alkylenyl)ₘ-; (C₁-C₆ alkyl)-C(O)—N(H)—S(O)₂—(C₁-C₈ alkylenyl)ₘ-; Halo; HO₂C; and ═O.

The compound according to any one of the preceding aspects, or a pharmaceutically acceptable salt thereof, wherein G¹ and G² are as defined above except each m is 1 and each C₁-C₈ alkylenyl is independently CH₂, CHF, CF₂, or C(═O).

The compound according to any one of the preceding aspects, or a pharmaceutically acceptable salt thereof, wherein G¹ and G² are as defined above except each m is 1 and each C₁-C₈ alkylenyl is independently CH₂, CHF, CF₂, or C(═O); R⁴ is H or fluoro, and R⁵ is H.

10. A compound of Formula I, or a pharmaceutically acceptable salt thereof, wherein D is selected from:

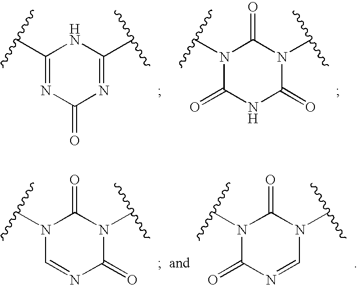

11. A compound of Formula I, or a pharmaceutically acceptable salt thereof, wherein D is selected from:

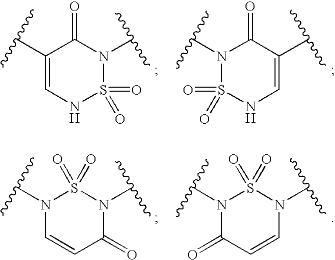

12. A compound of Formula I, or a pharmaceutically acceptable salt thereof, wherein D is selected from:

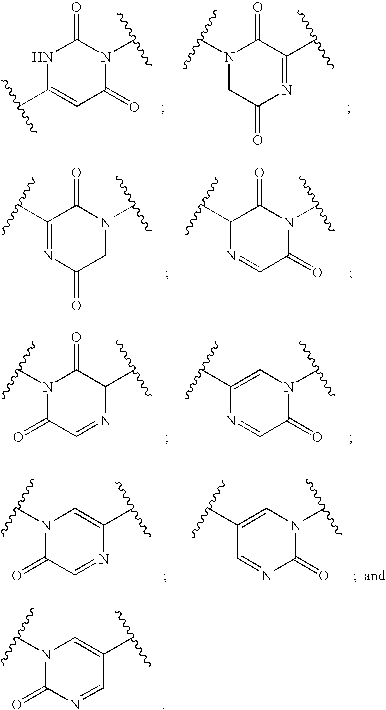

13. A compound of Formula I, or a pharmaceutically acceptable salt thereof, wherein D is selected from:

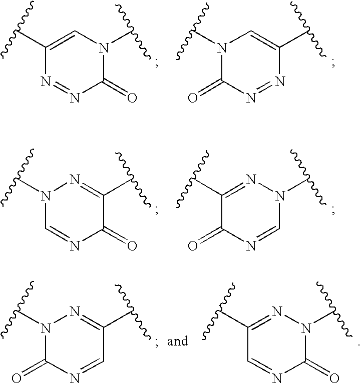

14. A compound of Formula I, or a pharmaceutically acceptable salt thereof, wherein D is selected from:

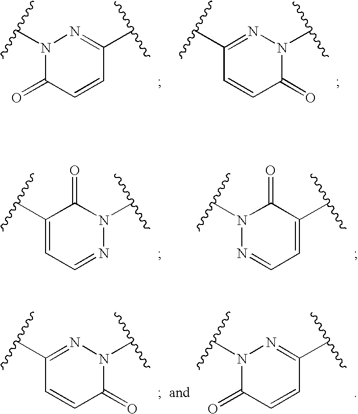

15. A compound of Formula I, or a pharmaceutically acceptable salt thereof, wherein D is selected from:

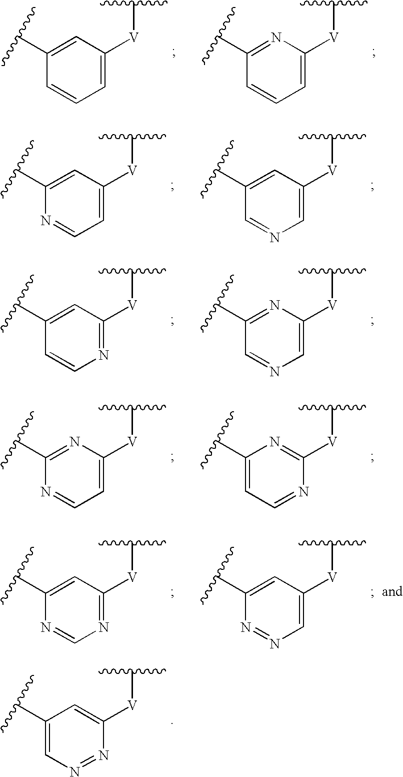

16. A compound of Formula I, or a pharmaceutically acceptable salt thereof, wherein D is selected from:

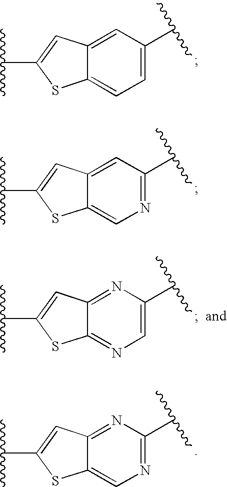

17. A compound of Formula I, or a pharmaceutically acceptable salt thereof, wherein D is selected from:

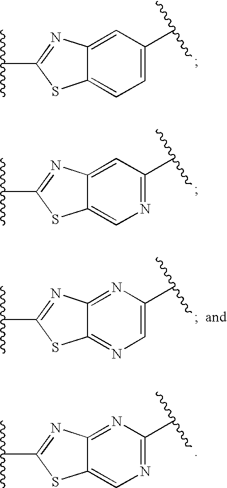

18. A compound of Formula I, or a pharmaceutically acceptable salt thereof, wherein D is selected from:

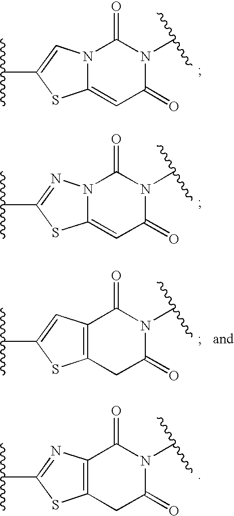

19. A compound of Formula I, or a pharmaceutically acceptable salt thereof, wherein D is selected from:

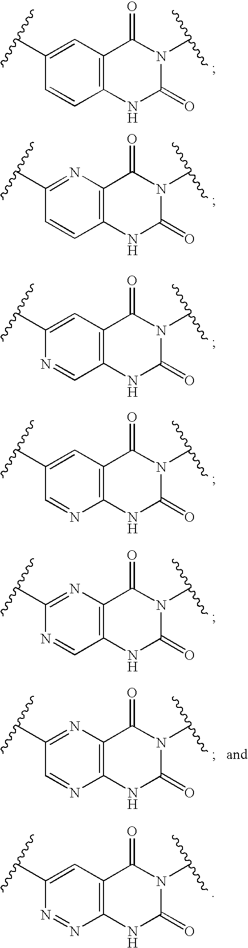

20. A compound of Formula I, or a pharmaceutically acceptable salt thereof, wherein D is selected from:

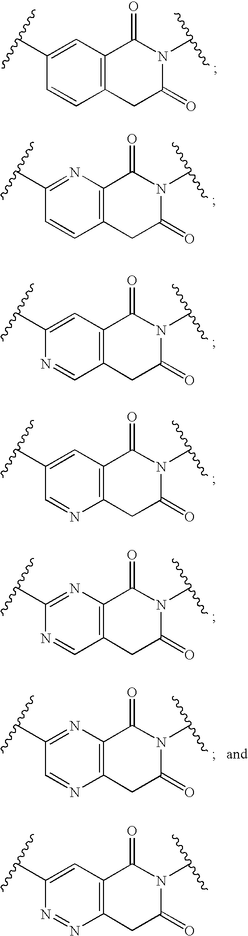

21. A compound of Formula I, or a pharmaceutically acceptable salt thereof, wherein D is selected from:

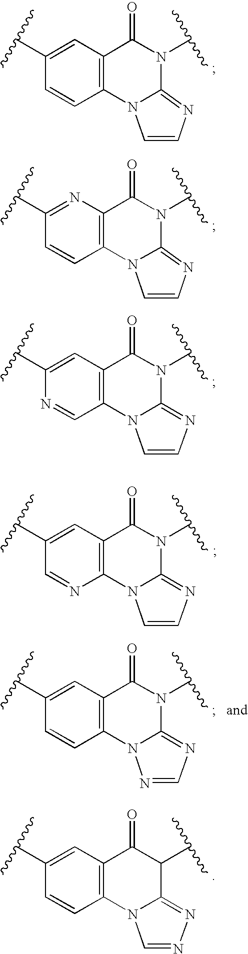

22. A compound of Formula I, or a pharmaceutically acceptable salt thereof, wherein D is selected from:

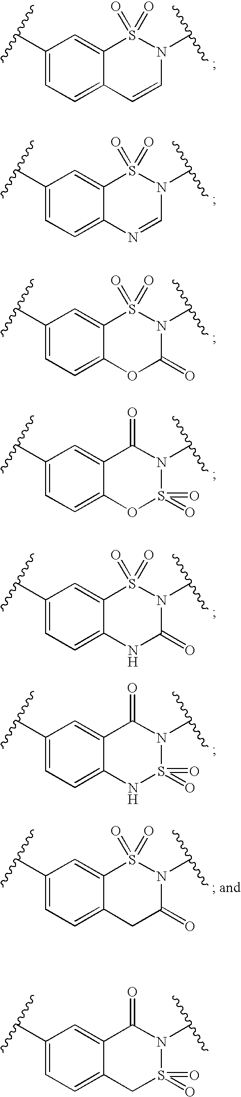

23. A compound of Formula I, or a pharmaceutically acceptable salt thereof, wherein D is selected from:

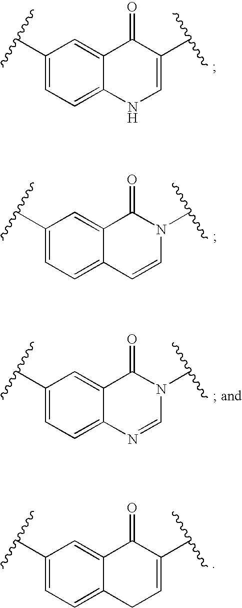

24. A compound of Formula I, or a pharmaceutically acceptable salt thereof, wherein D is selected from:

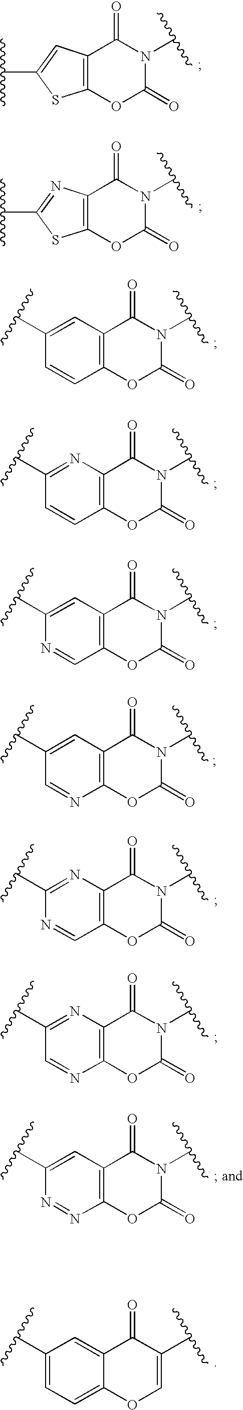

25. A compound of Formula I, or a pharmaceutically acceptable salt thereof, wherein D is selected from:

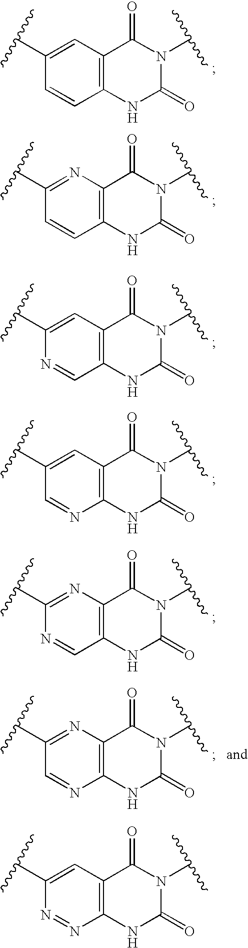

26. A compound of Formula I, or a pharmaceutically acceptable salt thereof, wherein D is selected from:

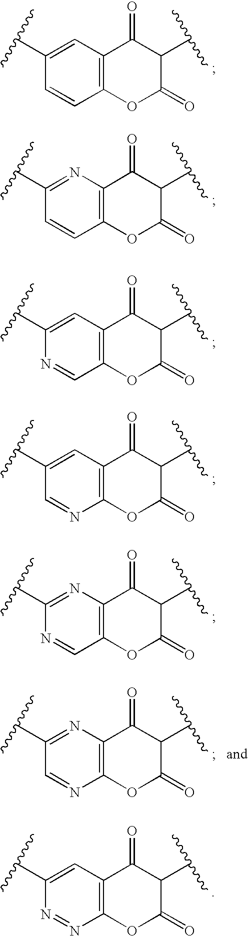

27. A compound of Formula I, or a pharmaceutically acceptable salt thereof, wherein D is selected from:

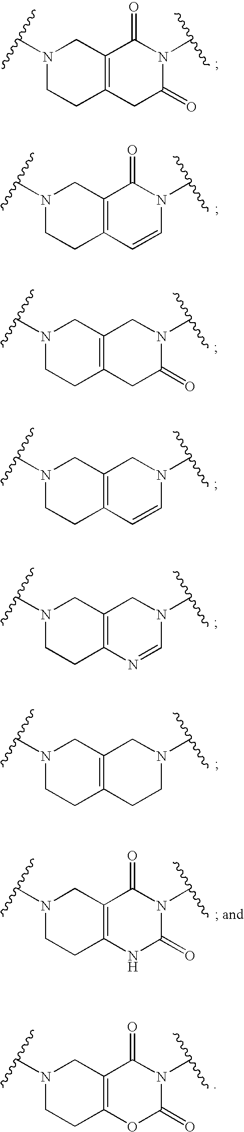

28. A compound of Formula I, or a pharmaceutically acceptable salt thereof, wherein D is selected from:

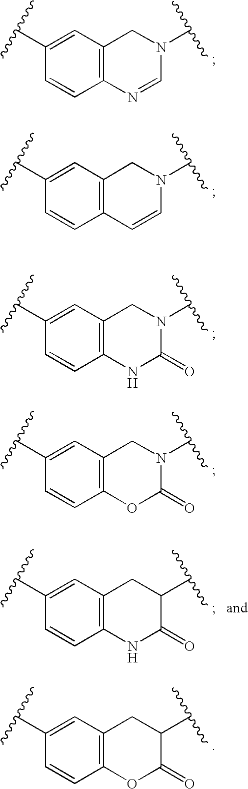

29. A compound of Formula I, or a pharmaceutically acceptable salt thereof, wherein D is selected from:

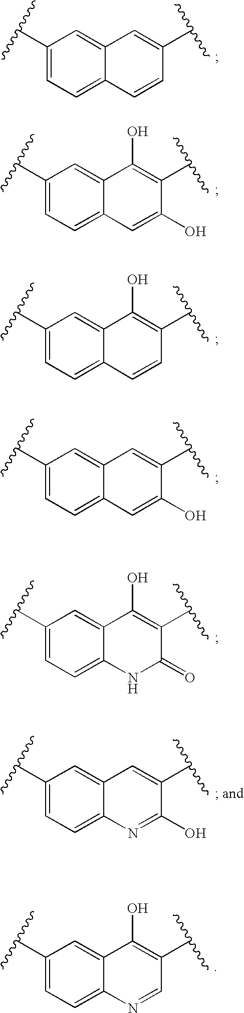

30. A compound of Formula I, or a pharmaceutically acceptable salt thereof, wherein D is selected from:

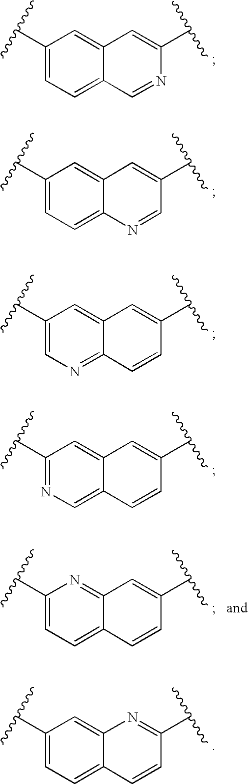

31. A compound of Formula I, or a pharmaceutically acceptable salt thereof, wherein D is selected from:

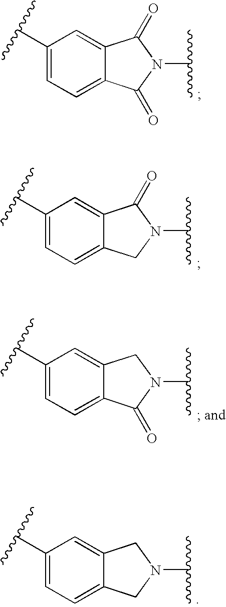

31. A compound of Formula I, or a pharmaceutically acceptable salt thereof, wherein D is selected from:

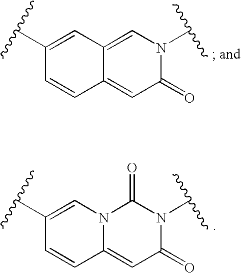

The compound according to any one of Formulas I to IV, or a pharmaceutically acceptable salt thereof, wherein V is selected from the groups:

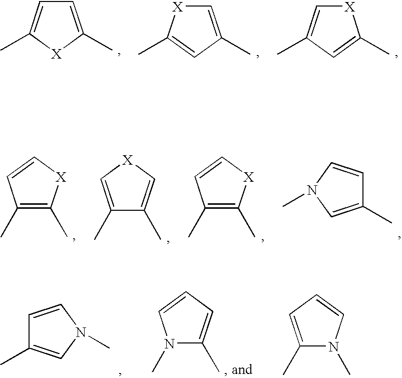

wherein X is O, S, N(H), or N(C₁-C₆ alkyl) and V may optionally be unsubstituted or substituted at C(H) or N(H) with 1 substituent selected from fluoro, methyl, hydroxy, trifluoromethyl, cyano, and acetyl.

The compound according to any one of Formulas I to IV, or a pharmaceutically acceptable salt thereof, wherein V is selected from the groups:

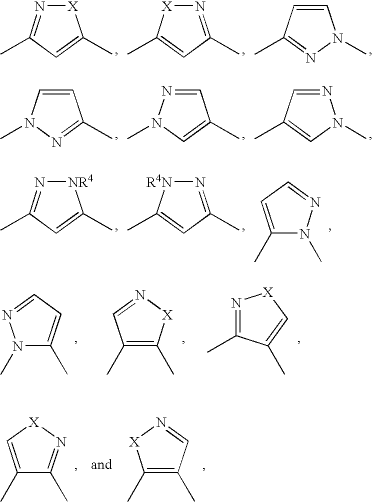

wherein X is O, S, N(H), or N(C₁-C₆ alkyl), R⁴ is H or C₁-C₆ alkyl, and V may optionally be unsubstituted or substituted at C(H) or N(H) with 1 substituent selected from fluoro, methyl, hydroxy, trifluoromethyl, cyano, and acetyl.

The compound according to any one of Formulas I to IV, or a pharmaceutically acceptable salt thereof, wherein V is selected from the groups:

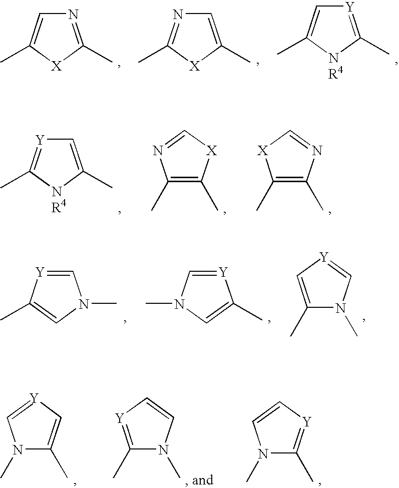

wherein X is O, S, N(H), or N(C₁-C₆ alkyl), Y is O, S, or N, and R⁴ is H or C₁-C₆ alkyl, and V may optionally be unsubstituted or substituted at C(H) or N(H) with 1 substituent selected from fluoro, methyl, hydroxy, trifluoromethyl, cyano, and acetyl.

The compound according to any one of Formulas I to IV, or a pharmaceutically acceptable salt thereof, wherein V is selected from the groups:

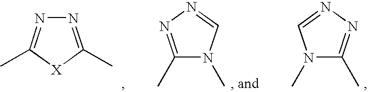

wherein X is O, S, N(H), or N(C₁-C₆ alkyl), and V may optionally be unsubstituted or substituted at C(H) with 1 substituent selected from fluoro, methyl, hydroxy, trifluoromethyl, cyano, and acetyl.

The compound according to any one of Formulas I to IV, or a pharmaceutically acceptable salt thereof, wherein V is selected from the groups:

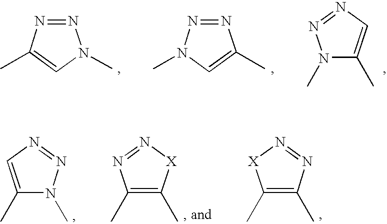

wherein X is O, S, N(H), or N(C₁-C₆ alkyl), and V may optionally be unsubstituted or substituted at C(H) with 1 substituent selected from fluoro, methyl, hydroxy, trifluoromethyl, cyano, and acetyl.

The compound according to any one of Formulas I to IV, or a pharmaceutically acceptable salt thereof, wherein V is selected from the groups:

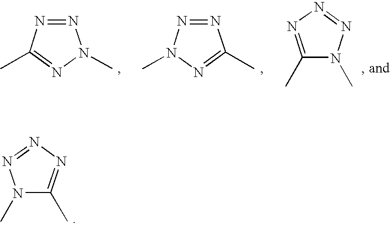

The compound according to any one of Formulas I to IV, or a pharmaceutically acceptable salt thereof, wherein instead of V is a 5-membered heteroarylenyl diradical, V is C(O)N(R⁵), wherein R⁵ is H or CH₃.

The compound according to any one of Formulas I to IV, or a pharmaceutically acceptable salt thereof, wherein instead of V is a 5-membered heteroarylenyl diradical, V is C(O)O.

32. The compound according to any one of Embodiments 1 to 31, or a pharmaceutically acceptable salt thereof, wherein G¹ is 5- or 6-membered heteroaryl-CH₂, 8- to 10-membered heterobiaryl-CH₂, or a substituted phenyl-CH₂; wherein 5- or 6-membered heteroaryl-CH₂ and 8- to 10-membered heterobiaryl-CH₂ may be independently unsubstituted or substituted as described above for Formula I.

33. The compound according to any one of Embodiments 1 to 31, or a pharmaceutically acceptable salt thereof, wherein G² is 5- or 6-membered heteroaryl-CH₂, 8- to 10-membered heterobiaryl-CH₂, or a substituted phenyl-CH₂; wherein 5- or 6-membered heteroaryl-CH₂ and 8- to 10-membered heterobiaryl-CH₂ may be independently unsubstituted or substituted as described above for Formula I.

34. The compound according to any one of Embodiments 1 to 31, or a pharmaceutically acceptable salt thereof, wherein:

G¹ and G² each independently are 5- or 6-membered heteroaryl-CH₂, 8- to 10-membered heterobiaryl-CH₂, or a substituted phenyl-CH₂; wherein 5- or 6-membered heteroaryl-CH₂ and 8- to 10-membered heterobiaryl-CH₂ may be independently unsubstituted or substituted as described above for Formula I.

35. The compound according to any one of Embodiments 1 to 31, or a pharmaceutically acceptable salt thereof, wherein:

Each G¹ and G² independently are 4-methoxyphenylmethyl,

3-methoxyphenylmethyl, 4-fluorophenylmethyl,

3-fluorophenylmethyl, 4-chlorophenylmethyl,

3-chlorophenylmethyl, 4-bromophenylmethyl,

3-bromophenylmethyl, 4-nitrophenylmethyl, 3-nitrophenylmethyl,

4-methylsulfanylphenylmethyl, 3-methylsulfanylphenylmethyl,

4-methylphenylmethyl, 3-methylphenylmethyl,

4-cyanophenylmethyl, 3-cyanophenylmethyl,

4-carboxyphenylmethyl, 3-carboxyphenylmethyl,

4-methanesulfonylphenylmethyl, 3-methanesulfonylphenylmethyl,

pyridin-4-ylmethyl, pyridin-3-ylmethyl, or pyridin-2-ylmethyl, or

2-methoxypyridin-4-ylmethyl.

36. A compound selected from:

4-{6-[2-(4-Methoxy-phenyl)-acetylamino]-1-methyl-2,4-dioxo-1,4-dihydro-2H-quinazolin-3-ylmethyl}-benzoic acid;

4-{6-[2-(3-Methoxy-phenyl)-acetylamino]-1-methyl-2,4-dioxo-1,4-dihydro-2H-quinazolin-3-ylmethyl}-benzoic acid;

4-{6-[2-(4-Fluoro-phenyl)-acetylamino]-1-methyl-2,4-dioxo-1,4-dihydro-2H-quinazolin-3-ylmethyl}-benzoic acid; and

4-{6-[2-(3-Fluoro-phenyl)-acetylamino]-11-methyl-2,4-dioxo-1,4-dihydro-2H-quinazolin-3-ylmethyl}-benzoic acid; or

a pharmaceutically acceptable salt thereof.

37. A compound selected from

4-{7-Fluoro-6-[2-(3-methoxy-phenyl)-acetoxy]-4-oxo-4H-quinazolin-3-ylmethyl}-benzoic acid;

4-{6-[2-(3-Methoxy-phenyl)-acetoxy]-4-oxo-4H-quinazolin-3-ylmethyl}-benzoic acid;

4-{6-[2-(4-Methoxy-phenyl)-acetoxy]-4-oxo-4H-quinazolin-3-ylmethyl}-benzoic acid;

4-{6-[2-(3-Fluoro-phenyl)-acetoxy]-4-oxo-4H-quinazolin-3-ylmethyl}-benzoic acid;

4-{6-[2-(4-Fluoro-phenyl)-acetoxy]-4-oxo-4H-quinazolin-3-ylmethyl}-benzoic acid;

4-{7-[2-(4-Fluoro-phenyl)-acetoxy]-1-oxo-1H-isoquinolin-2-ylmethyl}-benzoic acid;

4-{7-[2-(3-Fluoro-phenyl)-acetoxy]-1-oxo-1H-isoquinolin-2-ylmethyl}-benzoic acid;

4-{7-[2-(4-Methoxy-phenyl)-acetoxy]-1-oxo-1H-isoquinolin-2-ylmethyl}-benzoic acid;

4-{7-[2-(3-Methoxy-phenyl)-acetoxy]-1-oxo-1H-isoquinolin-2-ylmethyl}-benzoic acid;

4-{6-[2-(4-Methoxy-phenyl)-acetoxy]-1-methyl-2,4-dioxo-1,4-dihydro-2H-quinazolin-3-ylmethyl}-benzoic acid;

4-{6-[2-(3-Methoxy-phenyl)-acetoxy]-1-methyl-2,4-dioxo-1,4-dihydro-2H-quinazolin-3-ylmethyl}-benzoic acid;

4-{6-[2-(4-Fluoro-phenyl)-acetoxy]-1-methyl-2,4-dioxo-1,4-dihydro-2H-quinazolin-3-ylmethyl}-benzoic acid; and

4-{6-[2-(3-Fluoro-phenyl)-acetoxy]-1-methyl-2,4-dioxo-1,4-dihydro-2H-quinazolin-3-ylmethyl}-benzoic acid; or

a pharmaceutically acceptable salt thereof.

38. A pharmaceutical composition, comprising a compound according to any one of the above Embodiments 5 to 37 and the below Compound Examples A1 to A8 and B1 to B4, or a pharmaceutically acceptable salt thereof, admixed with a pharmaceutically acceptable carrier, excipient, or diluent.

39. A method for inhibiting an MMP-13 enzyme in an animal, comprising administering to the animal an MMP-13 inhibiting amount of a compound according to any one of the above Embodiments 5 to 37 and the below Compound Examples A1 to A8 and B1 to B4, or a pharmaceutically acceptable salt thereof.

40. A method for treating a disease mediated by an MMP-13 enzyme, comprising administering to a patient suffering from such a disease an effective amount of a compound according to any one of the above Embodiments 5 to 37 and the below Compound Examples A1 to A8 and B1 to B4, or a pharmaceutically acceptable salt thereof.

41. The method according to any one of Embodiments 4 and 40, wherein the disease is selected from breast carcinoma, rheumatoid arthritis, cartilage damage, and osteoarthritis.

42. The method according to any one of Embodiments 4 and 40, wherein the disease is selected from a rheumatic disease, gout, juvenile arthritis, ankylosing spondylitis, reactive arthritis (i.e., Reiter's arthritis), inflammatory bowel disease, and psoriasis.

43. The method according to any one of Embodiments 4 and 40, wherein the disease is selected from heart failure, cardiac insufficiency, heart disease, and atherosclerosis.

44. The method according to any one of Embodiments 4 and 40, wherein the disease is selected from inflammation and chronic obstructive pulmonary disease.

45. The method according to any one of Embodiments 4 and 40, wherein the disease is selected from a cancer mediated by MMP-13 other than breast cancer.

46. The method according to any one of Embodiments 4 and 40, wherein the disease is selected from age-related macular degeneration and osteoporosis.

47. The method according to any one of Embodiments 4 and 40, wherein the disease is selected from multiple sclerosis and a periodontal disease.

48. The method according to any one of Embodiments 4 and 40, wherein the disease is selected from an autoimmune disease, systemic lupus erythematosus, and asthma.

Use of a compound of Formula I, or a pharmaceutically acceptable salt thereof, in the preparation of a medicament useful for treating a disease selected from rheumatoid arthritis, osteoarthritis, breast cancer, heart failure, and atherosclerotic plaque rupture.

### DETAILED DESCRIPTION OF THE INVENTION

As described above, this invention provides a compound of Formula I

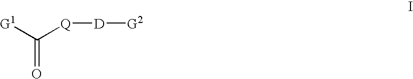

or a pharmaceutically acceptable salt thereof, wherein G¹, Q, D, and G² are as defined above for Formula I. Also described above are a pharmaceutical composition comprising a compound of Formula I, or a pharmaceutically acceptable salt thereof, and methods of inhibiting an MMP-13 enzyme in an animal and treating a disease mediated by an MMP-13 enzyme.

Additional Embodiments:

Nonlimiting examples of additional invention embodiments are described below.

Some of the invention compounds are capable of further forming pharmaceutically acceptable salts, including, but not limited to, acid addition and/or base salts. The acid addition salts are formed from basic invention compounds, whereas the base addition salts are formed from acidic invention compounds. All of these salt forms that are sufficiently nontoxic to a patient at therapeutic doses are within the scope of the compounds useful in the invention.

Useful pharmaceutically acceptable acid addition salts of the basic invention compounds include salts derived from inorganic acids such as hydrochloric, nitric, phosphoric, sulfuric, hydrobromic, hydroiodic, hydrofluoric, phosphorous, and the like, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66:1).

An acid addition salt of a basic invention compound is prepared by contacting the free base form of the compound with a sufficient amount of a desired acid to produce a sufficiently nontoxic salt in the conventional manner. The free base form of the compound may be regenerated by contacting the acid addition salt so formed with a base, and isolating the free base form of the compound in the conventional manner. The free base forms of compounds prepared according to a process of the present invention differ from their respective acid addition salt forms somewhat in certain physical properties such as solubility, crystal structure, hygroscopicity, and the like, but otherwise free base forms of the invention compounds and their respective acid addition salt forms are equivalent for purposes of the present invention.

Useful pharmaceutically acceptable base addition salts of acidic invention compounds include those comprising inorganic cations such as sodium cation (Na⁺), potassium cation (K⁺), magnesium cation (Mg²⁺), calcium cation (Ca²⁺), and the like and those comprising organic cations derived from an organic amine such as N,N′-dibenzylethylenediamine, chloroprocaine, choline, diethanolamine, dicyclohexylamine, ethylenediamine, N-methylglucamine, and procaine. A base addition salt of an acidic invention compound may be prepared by contacting the free acid form of the compound with the metal cation such as an alkali or alkaline earth metal cation, or the amine, especially an organic amine (see, for example, Berge, supra., 1977).

A base addition salt of an acidic invention compound may be prepared by contacting the free acid form of the compound with a sufficient amount of a desired base to produce the salt in the conventional manner. The free acid form of the compound may be regenerated by contacting the salt form so formed with an acid, and isolating the free acid of the compound in the conventional manner. The free acid forms of the invention compounds differ from their respective salt forms somewhat in certain physical properties such as solubility, crystal structure, hygroscopicity, and the like, but otherwise the salts are equivalent to their respective free acid for purposes of the present invention.

Certain invention compounds can exist in unsolvated forms as well as solvated forms, including hydrated forms. In general, the solvated forms, including hydrated forms, are equivalent to unsolvated forms and are encompassed within the scope of the present invention.

Certain invention compounds can exist as crystalline solids. Each invention compound capable of existing as a crystalline solid may crystallize in one or more polymorphic forms depending on the conditions used for crystallization. All polymorphic forms of crystalline invention compounds are encompassed within the scope of the present invention.

Certain of the invention compounds possess one or more chiral centers, and each center may exist in the R or S configuration. An invention compound includes any diastereomeric, enantiomeric, or epimeric form of the compound, as well as mixtures thereof.

Additionally, certain invention compounds may exist as geometric isomers such as the entgegen (E) and zusammen (Z) isomers of 1,2-disubstituted alkenyl groups or cis and trans isomers of disubstituted cyclic groups. An invention compound includes any cis, trans, syn, anti, entgegen (E), or zusammen (Z) isomer of the compound, as well as mixtures thereof.

Certain invention compounds can exist as two or more tautomeric forms. Tautomeric forms of the invention compounds are forms that may interchange by shifting of the position of a hydrogen atom and a bond(s), for example, via enolization/de-enolization, 1,2-hydride, 1,3-hydride, or 1,4-hydride shifts, and the like. Tautomeric forms of an invention compound are isomeric forms of the invention compound that exist in a state of equilibrium, wherein the isomeric forms of the invention compound have the ability to interconvert by isomerization in situ, including in a reaction mixture, in an in vitro biological assay, or in vivo. An invention compound includes any tautomeric form of the compound, as well as mixtures thereof.

Some compounds of the present invention have alkenyl groups, which may exist as entgegen or zusammen conformations, in which case all geometric forms thereof, both entgegen and zusammen, cis and trans, and mixtures thereof, are within the scope of the present invention.

Some compounds of the present invention have cycloalkyl groups, which may be substituted at more than one carbon atom, in which case all geometric forms thereof, both cis and trans, and mixtures thereof, are within the scope of the present invention.

It should be appreciated that a compound of Formula I, or a pharmaceutically acceptable salt thereof, or any form thereof as defined herein, that does not have an IC₅₀ with a human MMP-13 enzyme, determined according to any one of Biological Methods 1 to 10 described below, that is less than, or equal to, 100 micromolar, even though the compound falls within the scope of the genus described above for Formula I, is excluded from this invention. Preferred invention compounds have an IC₅₀ determined in Biological Method 1 or 5 with a human MMP-13 enzyme that is less than, or equal to, 10 micromolar, increasingly more preferably 1 micromolar, 100 nanomolar, and 10 nanomolar.

Further, another embodiment of the present invention is a compound of Formula I, or a pharmaceutically acceptable salt thereof, or any form thereof as defined herein, that is a selective inhibitor of the enzyme MMP-13. A selective inhibitor of MMP-13, as used in the present invention, is a compound that is ≧5 times, increasingly preferably ≧10, ≦20, ≧50, ≧100, or ≧1000, times more potent in vitro versus MMP-13 than versus at least one other matrix metalloproteinase enzyme such as, for example, MMP-1, MMP-2, MMP-3, MMP-7, MMP-8, MMP-9, MMP-12, MMP-14, or MMP-17 according one of the Biological Methods 1 to 10 described below. In other words, the IC₅₀ for the invention compound with an MMP-13 is ⅕, 1/10, 1/20, 1/50, 1/100, or 1/1000, respectively, of the IC₅₀ for the invention compound with the comparator MMP(s). A preferred aspect of the present invention is compounds that are selective inhibitors of MMP-13 versus MMP-1.

Another embodiment of the present invention is a compound of Formula I, or a pharmaceutically acceptable salt thereof, or any form thereof as defined herein, that is a highly selective inhibitor of an enzyme MMP-13. A highly selective inhibitor of MMP-13, as used in the present invention, is a compound that is at least ≧5, increasingly preferably ≧10, ≧20, ≧50, or ≧100, times more potent inhibitor of MMP-13 versus at least 3, preferably 4, increasingly more preferably 5, 6, or 7, 8, 9, or 10 of any other MMP enzymes. Preferably for purposes of determining invention compounds which are highly selective inhibitors of MMP-13, the comparator MMP enzymes are selected from MMP-1, MMP-2, MMP-3, MMP-7, MMP-8, MMP-9, MMP-12, MMP-14, and MMP-17.

Another embodiment of the present invention is a compound of Formula I, or a pharmaceutically acceptable salt thereof, or any form thereof as defined herein, that is a specific inhibitor of the enzyme MMP-13. A specific inhibitor of MMP-13, as used in the present invention, is a compound that is at least ≧5, increasingly preferably ≧10, ≧20, ≧50, or ≧100, times more potent inhibitor of MMP-13 versus at least 5, preferably 6, increasingly more preferably 7, 8, 9, or 10 of any other MMP enzymes. Preferably for purposes of determining invention compounds which are specific inhibitors of MMP-13, the comparator MMP enzymes are selected from MMP-1, MMP-3, MMP-7, MMP-8, MMP-9, MMP-12, MMP-14, and MMP-17.

For purposes of determining inhibitory selectivity or specificity of an invention compound with MMP-13, preferably, the MMP enzymes are human MMPs, including full length MMPs and catalytic domains thereof.

The invention compounds also include isotopically-labelled compounds, which are identical to those recited above, but for the fact that one or more atoms are replaced by an identical atom except the atom has an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention and pharmaceutically acceptable salts of said compounds which contain the aforementioned isotopes and/or other isotopes of other atoms are within the scope of this invention.

Certain isotopically labelled compounds of the present invention, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by carrying out the procedures incorporated by reference above or disclosed in the Schemes and/or in the Examples and Preparations below, by substituting a readily available isotopically labelled reagent for a non-isotopically labelled reagent.

One of ordinary skill in the art will appreciate that the compounds of the present invention are useful in treating a diverse array of diseases wherein inhibition of MMP-13 would be beneficial. One of ordinary skill in the art will also appreciate that when using the compounds of the invention in the treatment of a specific disease that the compounds of the invention may be combined with various existing therapeutic agents used for that disease.

Other mammalian diseases and disorders which are treatable by administration of an invention compound alone, an invention combination, or a pharmaceutical composition comprising the compound or combination as defined below, may include: rheumatic diseases such as arthritis, inflammatory skin diseases such as psoriasis, eczema, atopic dermatitis, discoid lupus, contact dermatitis, bullous pemphigoid, vulgaris, and alopecia areata, fever (including rheumatic fever and fever associated with influenza and other viral infections), common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache and toothache), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

For the treatment of rheumatoid arthritis, the compounds of the present invention may be combined with agents such as TNF-α inhibitors such as anti-TNF monoclonal antibodies such as adalimumab, which is known in the United States by the trade name HUMIRA®, and TNF receptor immunoglobulin molecules such as etanercept, which is marketed in the United States under the trade name Enbrel® for the treatment of rheumatoid arthritis, juvenile rheumatoid arthritis, and psoriatic arthritis, low dose methotrexate, lefunimide, hydroxychloroquine, d-penicillamine, auranofin or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, which is marketed in the United States under the trade name CELEBREX®, valdecoxib, which is marketed in the United States under the trade name BEXTRA®, parecoxib, etoricoxib, which is marketed in the United Kingdom under the trade name ARCOXIA®, and rofecoxib, which is marketed in the United States under the trade name VIOXX®, analgesics, and intraarticular therapies such as corticosteroids and hyaluronic acids such as hyalgan and synvisc.

This invention also relates to a method of or a pharmaceutical composition for treating inflammatory processes and diseases comprising administering a compound of this invention to a mammal, including a human, cat, livestock or dog, wherein said inflammatory processes and diseases are defined as above and said inhibitory compound is used in combination with one or more other therapeutically active agents under the following conditions:

A.) where a joint has become seriously inflamed as well as infected at the same time by bacteria, fungi, protozoa and/or virus, said inhibitory compound is administered in combination with one or more antibiotic, antifungal, antiprotozoal and/or antiviral therapeutic agents;

B.) where a multi-fold treatment of pain and inflammation is desired, said inhibitory compound is administered in combination with inhibitors of other mediators of inflammation, comprising one or more members independently selected from the group consisting essentially of:

(1) NSAIDs;

(2) H₁-receptor antagonists;

(3) kinin-B₁- and B₂-receptor antagonists;

(4) prostaglandin inhibitors selected from the group consisting of PGD-, PGF-PGI₂- and PGE-receptor antagonists;

(5) thromboxane A₂ (TXA₂-) inhibitors;

(6) 5-, 12- and 15-lipoxygenase inhibitors;

(7) leukotriene LTC₄—, LTD4/LTE₄- and LTB₄-inhibitors;

(8) PAF-receptor antagonists;

(9) gold in the form of an aurothio group together with one or more hydrophilic groups;

(10) immunosuppressive agents selected from the group consisting of cyclosporine, azathioprine and methotrexate;

(11) anti-inflammatory glucocorticoids;

(12) penicillamine;

(13) hydroxychloroquine;

(14) anti-gout agents including colchicine; xanthine oxidase inhibitors including allopurinol; and uricosuric agents selected from probenecid, sulfinpyrazone and benzbromarone;

C. where older mammals are being treated for disease conditions, syndromes and symptoms found in geriatric mammals, said inhibitory compound is administered in combination with one or more members independently selected from the group consisting essentially of:

(1) cognitive therapeutics to counteract memory loss and impairment;

(2) anti-hypertensives and other cardiovascular drugs intended to offset the consequences of atherosclerosis, hypertension, myocardial ischemia, angina, congestive heart failure and myocardial infarction, selected from the group consisting of:

a. diuretics;

b. vasodilators;

c. β-adrenergic receptor antagonists;

d. angiotensin-II converting enzyme inhibitors (ACE-inhibitors), alone or optionally together with neutral endopeptidase inhibitors;

e. angiotensin II receptor antagonists;

f. renin inhibitors;

g. calcium channel blockers;

h. sympatholytic agents;

i. α₂-adrenergic agonists;

j. α-adrenergic receptor antagonists; and

k. HMG-CoA-reductase inhibitors (anti-hypercholesterolemics);

(3) antineoplastic agents selected from:

a. antimitotic drugs selected from:

i. vinca alkaloids selected from:

[1] vinblastine and

[2] vincristine;

(4) growth hormone secretagogues;

(5) strong analgesics;

(6) local and systemic anesthetics; and

(7) H₂-receptor antagonists, proton pump inhibitors and other gastroprotective agents.

The active ingredient of the present invention may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leukotriene receptor antagonists, IL-1 processing and release inhibitors, ILra, H₁-receptor antagonists; kinin-B₁- and B₂-receptor antagonists; prostaglandin inhibitors such as PGD-, PGF-PGI₂- and PGE-receptor antagonists; thromboxane A₂ (TXA2-) inhibitors; 5- and 12-lipoxygenase inhibitors; leukotriene LTC₄-, LTD4/LTE₄- and LTB₄-inhibitors; PAF-receptor antagonists; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The compounds of the present invention may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The compounds of the present invention may also be used in combination with anti-hypertensives and other cardiovascular drugs intended to offset the consequences of atherosclerosis, including hypertension, myocardial ischemia including angina, congestive heart failure and myocardial infarction, selected from vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The compounds of the present invention may also be administered in combination with one or more antibiotic, antifungal, antiprotozoal, antiviral or similar therapeutic agents.

The compounds of the present invention may also be used in combination with CNS agents such as alpha-2-delta receptor ligands (such as gabapentin, pregabalin, or CI-1045), antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors such as those recited above, propentofylline or metryfonate.

The compounds of the present invention may also be used in combination with osteoporosis agents such as roloxifene, lasofoxifene, droloxifene or fosomax and immunosuppressant agents such as FK-506 and rapamycin.

The present invention also relates to the formulation of a compound of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

The invention method is useful in human and veterinary medicines for treating mammals suffering from one or more of the above-listed diseases and disorders. In humans, patients in need of treatment with an inhibitor of MMP-13 may be identified by a medical practitioner using conventional means. For example, patients at risk of having asymptomatic cartilage damage (e.g., osteoarthritis patients) may be identified clinically by assaying synovial fluid for the presence of MMP-13 catalyzed breakdown products from the extracellular matrix (for example, proteoglycans, type II cartilage, or hydroxyproline), specialized X-ray techniques, or nuclear magnetic resonance imaging (“MRI”) techniques. Patients at risk for osteoarthritis include elite athletes, laborers such as foundry workers, bus drivers, or coal miners, and patients with a family history of osteoarthritis. Further, patients presenting clinically with joint stiffness, pain, loss of joint function, or joint inflammation may be examined for cartilage damage using the above methods.

All that is required to practice a method of this invention is to administer to a patient a compound of Formula I, or a pharmaceutically acceptable salt thereof, in a sufficiently nontoxic amount that is therapeutically effective for preventing, inhibiting, or reversing the condition being treated. The invention compound can be administered directly or as part of a pharmaceutical composition.

Definitions:

As seen above, the groups of Formula I include “C₁-C₆ alkyl” groups. C₁-C₆ alkyl groups are straight and branched carbon chains having from 1 to 6 carbon atoms. Examples of C₁-C₆ alkyl groups include methyl, ethyl, 1-propyl, 2-propyl, 1-butyl, 2-butyl, 2,2-dimethylethyl, 1-pentyl, 2-pentyl, 2,2-dimethylpropyl, and 1-hexyl.

A substituted C₁-C₆ alkyl is a C₁-C₆ alkyl group as defined above that is substituted with from 1 to 6 substituents independently selected from the list above. Illustrative examples of substituted C₁-C₆ alkyl groups include CH₂OH, CF₂OH, CH₂C(CH₃)₂CO₂CH₃, CF₃, C(O)CF₃, C(O)—CH₃, (CH₂)₄—S—CH₃, CH(CO₂H)CH₂CH₂C(O)NMe₂, (CH₂)₅NH—C(O)—NH₂, CH₂—CH₂—C(H)-(4-fluorophenyl), CH(OCH₃)CH₂CH₃, CH₂SO₂NH₂, and CH(CH₃)CH₂CH₂OC(O)CH₃.

The term “C₃-C₇ cycloalkyl” means an unsubstituted cyclic hydrocarbon group having from 3 to 7 carbon atoms. C₃-C₇ cycloalkyl may optionally contain one carbon-carbon double bond. Similarly, a “C₅ or C₆ cycloalkyl” is an unsubstituted cyclic hydrocarbon group having 5 or 6 carbon atoms. The group C₃-C₇ cycloalkyl includes cyclopropyl, cyclobutyl, cyclopentyl, cyclopenten-1-yl, cyclopenten-4-yl, and cyclohexyl, the latter four groups also being C₅ or C₆ cycloalkyls.

A substituted C₃-C₇ cycloalkyl or substituted C₅ or C₆ cycloalkyl is a C₃-C₇ cycloalkyl or C₅ or C₆ cycloalkyl as defined above, respectively, which is substituted with from 1 to 6 substituents independently selected from the list above. Illustrative examples of substituted C₃-C₇ cycloalkyl groups include 1-hydroxy-cyclopropyl, cyclobutanon-3-yl, 3-(3-phenyl-ureido)-cyclopent-1-yl, and 4-carboxy-cyclohexyl, the latter two groups also being substituted C₅ or C₆ cycloalkyls.

The phrase “3- to 7-membered heterocycloalkyl” means an unsubstituted saturated cyclic group having carbon atoms and 1 or 2 heteroatoms independently selected from 2 O, 1 S, 1 S(O), 1 S(O)₂, 1 N, 2 N(H), and 2 N(C₁-C₆ alkyl), wherein when two O atoms or one O atom and one S atom are present, the two O atoms or one O atom and one S atom are not bonded to each other. Optionally, a 3- to 7-membered heterocycloalkyl may contain one carbon-carbon or carbon-nitrogen double bond. 5- or 6-membered heterocycloalkyl is similarly defined. Illustrative examples of 3- to 7-membered heterocycloalkyl includes aziridin-1-yl, 1-oxa-cyclobutan-2-yl, tetrahyrdofuran-3-yl, morpholin-4-yl, 2-thiacyclohex-1-yl, 2-oxo-2-thiacyclohex-1-yl, 2,2-dioxo-2-thiacyclohex-1-yl, and 4-methyl-piperazin-2-yl, the latter six groups also being 5- or 6-membered heterocycloalkyls.

A substituted 3- to 7-membered heterocycloalkyl or substituted 5- or 6-membered heterocycloalkyl is a 3- to 7-membered heterocycloalkyl or 5- or 6-membered heterocycloalkyl as defined above, respectively, which is substituted with from 1 to 6 substituents independently selected from the list above. Illustrative examples of substituted 3- to 7-membered heterocycloalkyl include 2-hydroxy-aziridin-1-yl, 3-oxo-1-oxacyclobutan-2-yl, 2,2-dimethyl-tetrahydrofuran-3-yl, 3-carboxy-morpholin-4-yl, and 1-cyclopropyl-4-methyl-piperazin-2-yl, the latter three groups also being substituted 5- or 6-membered heterocycloalkyls.

The phrase “C₈-C₁₀ bicycloalkyl” means a cyclopentyl or cyclohexyl fused to another cyclopentyl or cyclohexyl to give a 5,5-, 5,6-, or 6,6-fused bicyclic carbocyclic group, wherein the bicycloalkyl optionally contains 1 carbon-carbon double bond.

The phrase “5- or 6-membered heterocycloalkyl” means a 5- or 6-membered ring containing carbon atoms and 1 or 2 heteroatoms selected from 1 O, 1 S, 1 N, 2 N(H), and 2 N(C₁-C₆ alkyl).

The phrase “3- to 7-membered heterocycloalkyl” means a 3- to 7-membered heterocycloalkyl containing carbon atoms and 1 or 2 heteroatoms selected from 1 O, 1 S, 1 N, 2 N(H), and 2 N(C₁-C₆ alkyl).

The phrase “8- to 10-membered heterobicycloalkyl” means a 5- or 6-membered ring fused to another 5- or 6-membered ring to give a 5,5-, 5,6-, or 6,6-fused bicyclic group containing carbon atoms and from 1 to 4 heteroatoms independently selected from 2 O, 1 S, 1 S(O), 1 S(O)₂, 1 N, 4 N(H), and 4 N(C₁-C₆ alkyl), wherein the bicycloalkyl optionally contains 1 carbon-carbon double bond or 1 carbon-nitrogen double bond.

The term “naphthyl” includes 1-naphthyl and 2-napthyl.

The phrase “5- or 6-membered heteroaryl” means a 5-membered, monocyclic heteroaryl having carbon atoms and from 1 to 4 heteroatoms independently selected from 1 O, 1 S, 1 N(H), 1 N(C₁-C₆ alkyl), and 4 N, or a 6-membered, monocyclic heteroaryl having carbon atoms and 1 or 2 heteroatoms selected from 2 N, and wherein:

(i) The phrase “5-membered, monocyclic heteroaryl” means a 5-membered, monocyclic, aromatic ring group as defined above having carbon atoms and from 1 to 4 heteroatoms selected from 1 O, 1 S, 1 N(H), 1 N(C₁-C₆ alkyl), and 4 N. Illustrative examples of a 5-membered, monocyclic heteroaryl include thiophen-2-yl, furan-2-yl, pyrrol-3-yl, pyrrol-1-yl, imidazol-4-yl, isoxazol-3-yl, oxazol-2-yl, thiazol-4-yl, tetrazol-1-yl, 1,2,4-oxadiazol-3-yl, 1,2,4-triazol-1-yl, and pyrazol-3-yl; and

(ii) The phrase “6-membered, monocyclic heteroaryl” means a 6-membered, monocyclic, aromatic ring group as defined above having carbon atoms and 1 or 2 N. Illustrative examples of a 6-membered, monocyclic heteroaryl include pyridin-2-yl, pyridin-4-yl, pyrimidin-2-yl, pyridazin-4-yl, and pyrazin-2-yl.

The phrase “8- to 10-membered heterobiaryl” means an 8-membered, 5,5-fused bicyclic heteroaryl, a 9-membered, 6,5-fused bicyclic heteroaryl, or a 10-membered, 6,6-fused bicyclic heteroaryl, having carbon atoms and from 1 to 4 heteroatoms independently selected from 1 O, 1 S, 1 N(H), 1 N(C₁-C₆ alkyl), and 4 N, wherein at least one of the 2 fused rings is aromatic, and wherein when the O and S atoms both are present, the O and S atoms are not bonded to each other, which are as defined below:

(i) The phrase “8-membered, 5,5-fused bicyclic heteroaryl” means a an 8-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of an 8-membered, fused-bicyclic heteroaryl include

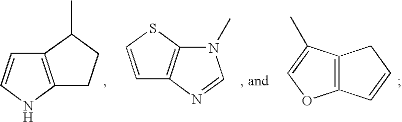

(ii) The phrase “9-membered, 6,5-fused bicyclic heteroaryl” means a 9-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 9-membered, fused-bicyclic heteroaryl include indol-2-yl, indol-6-yl, iso-indol-2-yl, benzimidazol-2-yl, benzimidazol-1-yl, benztriazol-1-yl, benztriazol-5-yl, benzoxazol-2-yl, benzothiophen-5-yl, and benzofuran-3-yl; and

(iii) The phrase “10-membered, 6,5-fused bicyclic heteroaryl” means a 10-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 10-membered, fused-bicyclic heteroaryl include quinolin-2-yl, isoquinolin-7-yl, and benzopyrimidin-2-yl.

The term “phenylenyl” means a diradical group derived from benzene by removing any two hydrogen atoms.

The phrase “5-membered heteroarylenyl” means a 5-membered monocyclic aromatic ring diradical containing carbon atoms and from 1 to 4 heteroatoms independently selected from 1 O, 1 S, 1 N(H), 1 N(C₁-C₆ alkyl), and 4 N. Illustrative examples of 5-membered heteroarylenyl include isoxazol-3,5-diyl, thiazol-2,4-diyl, and tetrazol-2,5-diyl.

The phrase “5- or 6-membered heteroarylenyl” means a 5-membered monocyclic aromatic ring diradical containing carbon atoms and from 1 to 4 heteroatoms independently selected from 1 O, 1 S, 1 N(H), 1 N(C₁-C₆ alkyl), and 4 N or a 6-membered monocyclic aromatic ring diradical containing carbon atoms and 1 or 2 heteroatoms selected from 2 N, respectively, wherein the 10 atom and the 1 S atom may not both be present in a ring. Illustrative examples of 5- or 6-membered heteroarylenyl include:

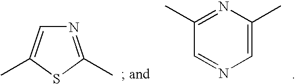

The phrase “8- to 10-membered heterobiarylenyl” means a diradical which is an 8-membered, 5,5-fused bicyclic heteroarylenyl, a 9-membered, 6,5-fused bicyclic heteroarylenyl, or a 10-membered, 6,6-fused bicyclic heteroarylenyl, having carbon atoms and from 1 to 4 heteroatoms independently selected from 1 O, 1 S, 1 N(H), 1 N(C₁-C₆ alkyl), and 4 N, wherein at least one of the 2 fused rings is aromatic, and wherein when the O and S atoms both are present, the O and S atoms are not bonded to each other, which are as defined below:

(i) The phrase “8-membered, 5,5-fused bicyclic heteroarylenyl” means a diradical which is an 8-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of an 8-membered, fused-bicyclic heteroarylenyl include

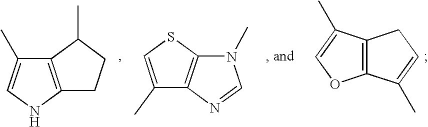

(ii) The phrase “9-membered, 6,5-fused bicyclic heteroarylenyl” means a diradical which is a 9-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 9-membered, fused-bicyclic heteroarylenyl include indol-2-yl, indol-6-yl, iso-indol-2-yl, benzimidazol-2,5-diyl, benzimidazol-1,2-diyl, and benzofuran-3,6-diyl; and

(iii) The phrase “10-membered, 6,5-fused bicyclic heteroarylenyl” means a diradical which is a 10-membered aromatic, fused-bicyclic ring group as defined above having carbon atoms and from 1 to 4 heteroatoms selected from 1 O, 1 S, 1 N(H), 1 N(C₁-C₆ alkyl), and 4 N. Illustrative examples of a 10-membered, fused-bicyclic heteroarylenyl include quinolin-2,5-diyl, isoquinolin-1,7-diyl, and benzopyrimidin-2,3-diyl.

The term “Biphenyl” means a phenyl bonded through a phenylenyl such as biphenyl-2-yl, biphenyl-3-yl, or biphenyl-4-yl.

The term “C₁-C₈ alkylenyl” means a saturated hydrocarbon diradical that is straight or branched and has from 1 to 8 carbon atoms or a saturated diradical having 1 heteroatom selected from O, S, S(O), S(O)₂, N(H), and N(CH₃), and from 0 to 7 carbon atoms. C₁-C₈ alkylenyl having from 2 to 8 atoms may optionally independently contain one carbon-carbon or carbon-nitrogen double bond. Illustrative examples of C₁-C₈ alkylenyl include CH₂, CH₂CH₂, C(CH₃)H, C(H)(CH₃)CH₂CH₂, CH₂C(H)═C(H)CH₂CH₂CH₂CH₂CH₂, O, S, S(O), S(O)₂, NH, N(CH₃), OCH₂, CH₂CH₂O, C(CH₃)HS, and CH₂C(H)═C(H)CH₂N(H)CH₂CH₂CH₂.

A substituted C₁-C₈ alkylenyl is a C₁-C₈ alkylenyl as defined above which is substituted with from 1 to 6 substituents independently selected from the list above. Illustrative examples of a substituted C₁-C₈ alkylenyl includes CF₂, C(O)CH₂, CH₂CH(CO₂H), CH₂CH₂OC(O), CF₂CH₂O, C(CH₃)(CN)SCH₂CH₂, and CH₂C(H)═C(H)CH₂N(OH).

The phrases “C₅ or C₆ cycloalkyl-(C₁-C₈ alkylenyl)”, “C₃-C₇ cycloalkyl-(C₁-C₈ alkylenyl)”, “C₈-C₁₀ bicycloalkyl-(C₁-C₈ alkylenyl)”, et cetera mean a C₅ or C₆ cycloalkyl, C₃-C₇ cycloalkyl, C₈-C₁₀ bicycloalkyl, et cetera as defined above, respectively, bonded through a C₁-C₈ alkylenyl, as defined above. Illustrative examples of C₅ or C₆ cycloalkyl-(C₁-C₈ alkylenyl), C₃-C₇ cycloalkyl-(C₁-C₈ alkylenyl), and C₈-C₁₀ bicycloalkyl-(C₁-C₈ alkylenyl) include 1-cyclopentyl-hex-2-yl; cyclopropylmethyl and 2-cyclobutyl-but-2-yl; and 3-bicyclo[2.2.2]octyl-propyl, respectively.

The phrases “5- or 6-membered heterocycloalkyl-phenylenyl-(C₁-C₈ alkylenyl)”, “5- or 6-membered heteroaryl-phenylenyl-(C₁-C₈ alkylenyl)”, 8- to 10-membered heterobiaryl-phenylenyl-(C₁-C₈ alkylenyl)”, et cetera mean a 5- or 6-membered heterocycloalkyl, 5- or 6-membered heteroaryl, 8- to 10-membered heterobiaryl, et cetera, as defined above, respectively, bonded through a phenylenyl diradical, as defined above, which is in turn bonded through a C₁-C₈ alkylenyl, as defined above.

The phrases “5- or 6-membered heteroaryl-(5- or 6-membered heteroarylenyl)-(C₁-C₈ alkylenyl)”, “Phenyl-(5- or 6-membered heteroarylenyl)-(C₁-C₈ alkylenyl)”, “Naphthyl-(5- or 6-membered heteroarylenyl)-(C₁-C₈ alkylenyl)”, et cetera, as defined above, respectively, mean a 5- or 6-membered heteroaryl, as defined above, phenyl, naphthyl, et cetera bonded through a 5- or 6-membered heteroarylenyl, as defined above, which is in turn bonded through a C₁-C₈ alkylenyl, as defined above.

The phrase “Phenyl-L-(5- or 6-membered heteroarylenyl)-(C₁-C₈ alkylenyl)” means a phenyl, as defined above, bonded through a linker group L, as defined above, which is in turn bonded through a 5- or 6-membered heteroarylenyl, as defined above, which is in turn bonded through a C₁-C₈ alkylenyl, as defined above.

The phrase “Phenyl-(8- to 10-membered heterobiarylenyl)-(C₁-C₈ alkylenyl)” means a phenyl, as defined above, bonded through a 8- to 10-membered heterobiarylenyl, as defined above, which is in turn bonded through a C₁-C₈ alkylenyl, as defined above.

The phrases “(C₁-C₆ alkyl)-O”, “(C₁-C₆ alkyl)-S”, “(C₁-C₆ alkyl)-S(O)”, “(C₁-C₆ alkyl)-S(O)₂”, and “(C₁-C₆ alkyl)-N(H)” mean a C₁-C₆ alkyl group, as defined above, bonded through an oxygen atom, sulfur atom, a sulfur atom that is substituted with one oxygen atom, a sulfur atom that is substituted with two oxygen atoms, or a secondary nitrogen atom, respectively.

The phrase “(C₁-C₆ alkyl)₂—N” means two independently selected C₁-C₆ alkyl groups, as defined above, including cyclic groups wherein the two C₁-C₆ alkyl groups are taken together with the nitrogen atom to which they are both bonded to form a 5- or 6-membered heterocycloalkyl, bonded through a nitrogen atom.

The phases “(C₁-C₆ alkyl)-C(O)O-(C₁-C₈ alkylenyl)” and “(C₁-C₆ alkyl)-C(O)O-(1- to 8-membered heteroalkylenyl)” mean a C₁-C₆ alkyl group, as defined above, bonded through a carbonyl carbon atom, bonded through an oxygen atom, bonded through a C₁-C₈ alkylenyl or 1- to 8-membered heteroalkylenyl, as defined above, respectively.

The phases “(C₁-C₆ alkyl)-C(O)N(H)—(C₁-C₈ alkylenyl)” and “(C₁-C₆ alkyl)-C(O)N(H)-(1- to 8-membered heteroalkylenyl)” mean a C₁-C₆ alkyl group, as defined above, bonded through a carbonyl carbon atom, bonded through a nitrogen atom, which is bonded to a hydrogen atom, bonded through a C₁-C₈ alkylenyl or 1- to 8-membered heteroalkylenyl, as defined above, respectively.

The phrases “(C₁-C₆ alkyl)-N(H)S(O)₂—(C₁-C₈ alkylenyl)” and “(C₁-C₆ alkyl)₂-NS(O)₂—(C₁-C₈ alkylenyl)” mean a C₁-C₆ alkyl or two independently selected C₁-C₆ alkyl groups, including cyclic groups wherein the two C₁-C₆ alkyl groups are taken together with the nitrogen atom to which they are both bonded to form a 5- or 6-membered heterocycloalkyl, as defined above, respectively, each bonded through the nitrogen atom, bonded through a sulfur atom, which in turn is bonded through a C₁-C₈ alkylenyl is as defined above.

The phrases “3- to 6-membered heterocycloalkyl-(G)” and “5- or 6-membered heteroaryl-(G)” mean a 3- to 6-membered heterocycloalkyl or 5- or 6-membered heteroaryl, as defined above, respectively, bonded through a group G, as defined above.

The phrases “Phenyl-O—(C₁-C₈ alkylenyl)”, “Phenyl-S—(C₁-C₈ alkylenyl)”, “Phenyl-S(O)—(C₁-C₈ alkylenyl)”, and “Phenyl-S(O)₂—(C₁-C₈ alkylenyl)” mean a phenyl bonded through an oxygen atom, sulfur atom, a sulfur atom that is also bonded to an oxygen atom, or a sulfur atom that is also bonded to two oxygen atoms, respectively, which in turn is bonded through a C₁-C₈ alkylenyl, wherein C₁-C₈ alkylenyl is as defined above.

The phrases “(C₁-C₆ alkyl)-S(O)₂—N(H)—C(O)—(C₁-C₈ alkylenyl)” and “(C₁-C₆ alkyl)-C(O)—N(H)—S(O)₂—(C₁-C₈ alkylenyl)” mean a C₁-C₆ alkyl group, as defined above, bonded through a sulfur atom that is substituted with two oxygen atoms, which is bonded through a nitrogen atom, which is bonded through a carbonyl carbon atom, which is in turn bonded through a C₁-C₈ alkylenyl group, as defined above, and a C₁-C₆ alkyl group, as defined above, bonded through a carbonyl carbon atom, which is bonded through a nitrogen atom, which is bonded through a sulfur atom that is substituted with two oxygen atoms, which are in turn bonded through a C₁-C₈ alkylenyl group, as defined above.

It should be appreciated that the groups defined above may be substituted with from 1 to 6 substituents as described above for Formula I. An above defined group which is substituted is a group that comprises a radical group, as defined above, and one or more diradical groups, such as those defined above, may be substituted on the radical group alone, on a diradical group alone, on both the radical group and on a diradical group, on two or more diradical groups, if present, or on the radical group and two or more of the diradical groups, if present. For illustration purposes, a substituted C₅ or C₆ cycloalkyl-(C₁-C₈ alkylenyl) is a substituted group comprised of a radical group which is a C₅ or C₆ cycloalkyl and one diradical group which is a C₁-C₈ alkylenyl. A substituted C₅ or C₆ cycloalkyl-(C₁-C₈ alkylenyl) may thus be substituted on either the C₅ or C₆ cycloalkyl alone, on the C₁-C₈ alkylenyl alone, or on both the C₅ or C₆ cycloalkyl and the C₁-C₈ alkylenyl, with from 1 to 6 substituents as described above for Formula I.

It should be appreciated that an above group that comprises, for example, a (radical group)-(C₁-C₈ alkylenyl)ₘ- or a (radical group)-(diradical group)-(C₁-C₈ alkylenyl)ₘ-, wherein m is an integer of 0 or 1, means the radical group or (radical group)-(diradical group), respectively, when m is 0 and the group (radical group)-(C₁-C₈ alkylenyl) or (radical group)-(diradical group)-(C₁-C₈ alkylenyl), respectively, when m is 1. For illustration, C₅ or C₆ cycloalkyl-(C₁-C₈ alkylenyl)ₘ- and 8- to 10-membered heterobiaryl-phenylenyl-(C₁-C₈ alkylenyl)ₘ-mean the groups C₅ or C₆ cycloalkyl and 8- to 10-membered heterobiaryl-phenylenyl, respectively, when m is 0 and the groups C₅ or C₆ cycloalkyl-(C₁-C₈ alkylenyl) and 8- to 10-membered heterobiaryl-phenylenyl-(C₁-C₈ alkylenyl), respectively, when m is 1.

Representative examples of substituted groups are provided below for illustration purposes. The examples of substituted groups are not meant to limit the description of the substituted invention compounds described above for Formula I in any way, but are merely provided for convenience.

Illustrative examples of naphthyl-(C₁-C₈ alkylenyl) include naphth-1-ylmethyl, 2-(naphth-1-yl)ethyl, and 3-(naphth-2-yl)-1-heptyl.

Illustrative examples of substituted phenyl-(C₁-C₈ alkylenyl) include 4-fluoro-phenylmethyl, 2-(4-carboxy-phenyl)-ethyl, 1-(2,4-dimethoxy-phenyl)-2-oxo-propyl, and 1-phenyl-5,5-difluoro-oct-3-yl.

Illustrative examples of substituted phenyl-(C₁-C₈ alkylenyl) include 4-fluoro-(naphth-1-yl)methyl, 2-(4-carboxy-(naphth-1-yl))-ethyl, 1-(2,4-dimethoxy-(naphth-1-yl))-2-oxo-propyl, and 1-(naphth-2-yl)-5,5-difluorohept-2-yl.

Illustrative examples of 5- or 6-membered heteroarylenyl-(C₁-C₈ alkylenyl) include:

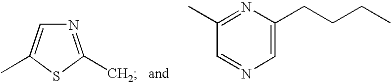

Illustrative examples of phenylenyl-(C₁-C₈ alkylenyl) include:

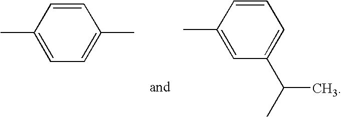

Illustrative examples of substituted 5-membered, monocyclic heteroaryl groups include 2-hydroxy-oxoazol-4-yl, 5-chloro-thiophen-2-yl, 1-methylimidazol-5-yl, 1-propyl-pyrrol-2-yl, 1-acetyl-pyrazol-4-yl, 1-methyl-1,2,4-triazol-3-yl, and 2-hexyl-tetrazol-5-yl.

Illustrative examples of substituted 6-membered, monocyclic heteroaryl groups include 4-acetyl-pyridin-2-yl, 3-fluoro-pyridin-4-yl, 5-carboxy-pyrimidin-2-yl, 6-tertiary butyl-pyridazin-4-yl, and 5-hdyroxymethyl-pyrazin-2-yl.

Illustrative examples of substituted 8-membered, 5,5-fused bicyclic heteroaryl include:

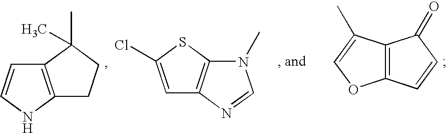

Illustrative examples of substituted 9-membered, 5,6-fused bicyclic heteroaryl include 3-(2-aminomethyl)-indol-2-yl, 2-carboxy-indol-6-yl, 1-(methanesulfonyl)-iso-indol-2-yl, 5-trifluorometyl-6,7-difluoro-4-hydroxymethyl-benzimidazol-2-yl, 4-(3-methylureido)-2-cyano-benzimidazol-1-yl, 1-methylbenzimidazol-6-yl, 1-acetylbenztriazol-7-yl, 1-methanesulfonyl-indol-3-yl, 1-cyano-6-aza-indol-5-yl, and 1-(2,6-dichlorophenylmethyl)-benzpyrazol-3-yl.

Illustrative examples of substituted 10-membered, 6,6-fused bicyclic heteroaryl include 5,7-dichloro-quinolin-2-yl, isoquinolin-7-yl-1-carboxylic acid ethyl ester, and 3-bromo-benzopyrimidin-2-yl.

Illustrative examples of substituted 5-membered heteroaryl-(C₁-C₈ alkylenyl) groups include 2-hydroxy-oxoazol-4-ylmethyl, 4-(5-chloro-thiophen-2-yl)-hex-1-yl, and 2-tetrazol-5-yloctyl.

Illustrative examples of substituted 6-membered heteroaryl-(C₁-C₈ alkylenyl) groups include 4-acetyl-pyridin-2-ylmethyl, 7-(3-fluoro-pyridin-4-yl)-hept-2-yl, and 2-(5-hdyroxymethyl-pyrazin-2-yl)-1,1-difluoro-2-hydroxy-prop-2-yl.

Illustrative examples of substituted 8-membered heterobiaryl-(C₁-C₈ alkylenyl) include:

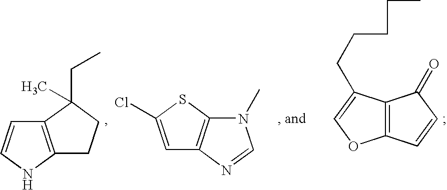

Illustrative examples of substituted 9-membered heterobiaryl-(C₁-C₈ alkylenyl) include 3-(2-aminomethyl)-indol-2-ylmethyl, and 1-(1-(2,6-dichlorophenylmethyl)-benzpyrazol-3-yl)-prop3-yl.

Illustrative examples of substituted 10-membered heterobiaryl-(C₁-C₈ alkylenyl) include 5,7-dichloro-quinolin-2-ylmethyl, and 5-(3-bromo-benzopyrimidin-2-yl)-oct-2-yl.

Illustrative examples of substituted phenyl-O—(C₁-C₈ alkylenyl) include 4-fluorophenoxymethyl and 2-phenoxy-methylcarbonyl.

Illustrative examples of substituted phenyl-S—(C₁-C₈ alkylenyl) include 4-fluorothiophenoxymethyl and 2-thiophenoxy-methylcarbonyl.

Illustrative examples of substituted phenyl-S(O)—(C₁-C₈ alkylenyl) include (4-Fluoro-phenyl)-S(═O)—CH₂ and phenyl-S(═O)—CH₂C(═O).

Illustrative examples of substituted phenyl-S(O)₂—(C₁-C₈ alkylenyl) include (4-Fluoro-phenyl)-S(═O)₂—CH₂ and phenyl-S(═O)₂—CH₂C(═O).

Illustrative examples of phenyl-O—(C₁-C₈ alkylenyl) include phenoxymethyl and 2-phenoxyethyl.

Illustrative examples of phenyl-S—(C₁-C₈ alkylenyl) include thiophenoxymethyl and 2-thiophenoxyethyl.

Illustrative examples of phenyl-S(O)—(C₁-C₈ alkylenyl) include phenyl-S(═O)—CH₂ and phenyl-S(═O)—CH₂CH₂.

Illustrative examples of phenyl-S(O)₂—(C₁-C₈ alkylenyl) include phenyl-S(═O)₂—CH₂ and phenyl-S(═O)₂—CH₂CH₂.

Illustrative examples of (C₁-C₆ alkyl)-S(═O)₂—N(H)—C(O)—(C₁-C₈ alkylenyl)ₘ include CH₃—S(O)₂—N(H)—C(═O) and CH₃—S(O)₂—N(H)—C(═O)—CH₂.

Illustrative examples of (C₁-C₆ alkyl)-C(O)—N(H)—S(O)₂—(C₁-C₈ alkylenyl)ₘ include CH₃—C(═O)—N(H)—S(═O)₂ and CH₃—C(═O)—N(H)—S(═O)₂—CH₂.

Preferred substituents for substituted phenyl, substituted naphthyl (i.e., substituted 1-naphthyl or substituted 2-naphthyl), and preferred substituents at carbon atoms for substituted 5-membered, monocyclic heteroaryl, substituted 6-membered, monocyclic heteroaryl, and substituted 9- or 10-membered, fused-bicyclic heteroaryl are C₁-C₄ alkyl, halo, OH, O—C₁-C₄ alkyl, 1,2-methylenedioxy, CN, NO₂, N₃, NH₂, N(H)CH₃, N(CH₃)₂, C(O)CH₃, OC(O)—C₁-C₄ alkyl, C(O)—H, CO₂H, CO₂—(C₁-C₄ alkyl), C(O)—N(H)OH, C(O)NH₂, C(O)NHMe, C(O)N(Me)₂, NHC(O)CH₃, N(H)C(O)NH₂, SH, S—C₁-C₄ alkyl, C≡CH, C(═NOH)—H, C(═NOH)—CH₃, CH₂OH, CH₂NH₂, CH₂N(H)CH₃, CH₂N(CH₃)₂, C(H)F—OH, CF₂—OH, S(O)₂NH₂, S(O)₂N(H)CH₃, S(O)₂N(CH₃)₂, S(O)—CH₃, S(O)₂CH₃, S(O)₂CF₃, or NHS(O)₂CH₃.

Especially preferred substituents are 1,2-methylenedioxy, methoxy, ethoxy, —O—C(O)CH₃, carboxy, carbomethoxy, and carboethoxy.

The term “1,2-methylenedioxy” means the diradical group —O—CH₂—O—, wherein the substituent 1,2-methylenedioxy is bonded to adjacent carbon atoms of the group which is substituted to form a 5-membered ring. Illustrative examples of groups substituted by 1,2-methylenedioxy include 1,3-benzoxazol-5-yl of formula B

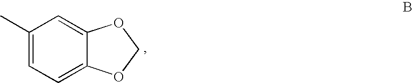

which is a phenyl group substituted by 1,2-methylenedioxy.

A fused-bicyclic group is a group wherein two ring systems share two, and only two, atoms.

It should be appreciated that the groups heteroaryl or heterocycloalkyl may not contain two ring atoms bonded to each other which atoms are oxygen and/or sulfur atoms.

The term “oxo” means ═O. Oxo is attached at a carbon atom unless otherwise noted. Oxo, together with the carbon atom to which it is attached forms a carbonyl group (i.e., C═O).

The term “heteroatom” includes O, S, S(O), S(O)₂, N, N(H), and N(C₁-C₆ alkyl).

The term “halo” includes fluoro, chloro, bromo, and iodo.

The term “amino” means NH₂.

The phrase “substitutable benzo carbon atom” means a carbon atom of a benzene ring that is fused to another ring, wherein the carbon atom is capable of being substituted with hydrogen or the group R⁴.

The phrase “ene carbon atom” means a carbon atom of a carbon-carbon double bond, wherein the carbon atom is capable of being substituted with hydrogen or the group R⁵.

The phrase “two adjacent, substantially sp² carbon atoms” means carbon atoms that comprise a carbon-carbon double bond that is capable of being substituted on each carbon atom, wherein the carbon-carbon double bond is contained in an aromatic or nonaromatic, cyclic or acyclic, or carbocyclic or heterocyclic group.

It should be appreciated that a 5- or 6-membered heteroaryl or an 8- to 10-membered heterobiaryl includes groups such as benzimidazolyl, benzofuranyl, benzofurazanyl, 2H-1-benzopyranyl, benzothiadiazine, benzothiazinyl, benzothiazolyl, benzothiophenyl, benzoxazolyl, chromanyl, cinnolinyl, furazanyl, furopyridinyl, indazolyl, indolinyl, indolizinyl, indolyl, 3H-indolyl, isoindolyl, isoquinolinyl, isothiazolyl, isoxazolyl, naphthyridinyl, oxadiazolyl, oxazolyl, phthalazinyl, pteridinyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, triazolyl, benzofuran, isobenzofuran, benzothiofuran, isobenzothiofuran, indole, indolenine, 2-isobenzazole, 1,5-pyrindine, pyrano[3,4-b]-pyrrole, isoindazole, indoxazine, benzoxazole, anthranil, benzopyran, coumarin, chromone, isocoumarin, 2,3-benzopyrone, quinoline, isoquinoline, cinnoline, quinazoline, naphthyridine, pyrido[3,4-b]-pyridine, pyrido[3,2-b]-pyridine, pyrido[4,3-b]pyridine, and benzoxazine, wherein said group may be optionally substituted on any of the ring carbon atom or nitrogen atom capable of forming an additional bond as described above. The foregoing groups, as derived from the compounds listed above, can be C-attached or N-attached where such is possible. For example, a group derived from pyrrole can be pyrrol-1-yl (N-attached) or pyrrol-4-yl (C-attached).

It should be appreciated that a 5-membered heteroarylenyl includes groups such as isothiazoldiyl, isoxazoldiyl, oxadiazoldiyl, oxazoldiyl, pyrazoldiyl, pyrroldiyl, tetrazoldiyl, thiazoldiyl, thiadiazoldiyl, thiendiyl, triazindiyl, triazoldiyl, and the like, wherein said group may be optionally substituted on any of the ring carbon atom or nitrogen atom capable of forming an additional bond as described above. The foregoing groups, as derived from the compounds listed above, can be C-attached or N-attached where such is possible. For example, a group derived from pyrrole can be pyrrol-1-yl (N-attached) or pyrrol-4-yl (C-attached).

Preferred is V which is a 5-membered heteroarylenyl diradicals selected from:

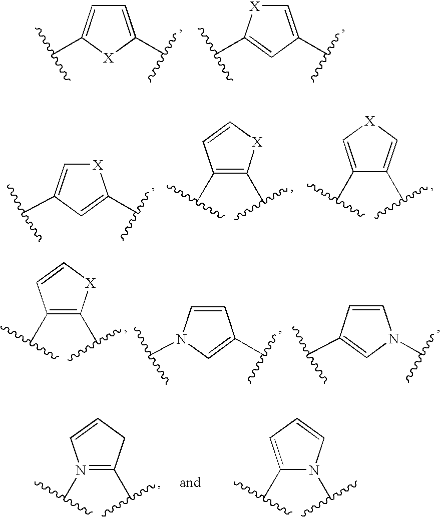

wherein X is O, S, N(H), or N(C₁-C₆ alkyl).

Also preferred is V which is a 5-membered heteroarylenyl diradicals selected from:

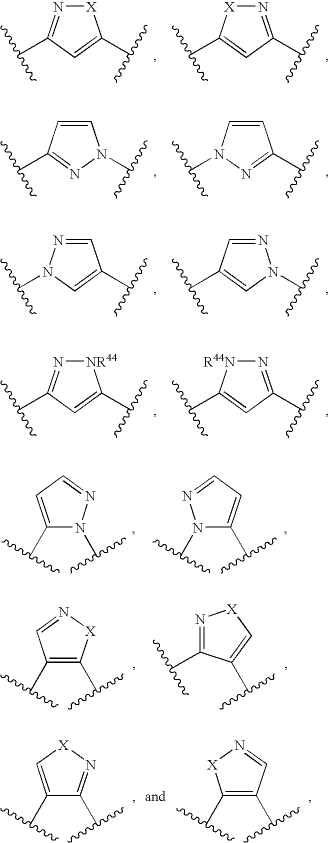

wherein X is O, S, N(H), or N(C₁-C₆ alkyl), R⁴⁴ is H or C₁-C₆ alkyl.

Also preferred is V which is a 5-membered heteroarylenyl diradicals selected from:

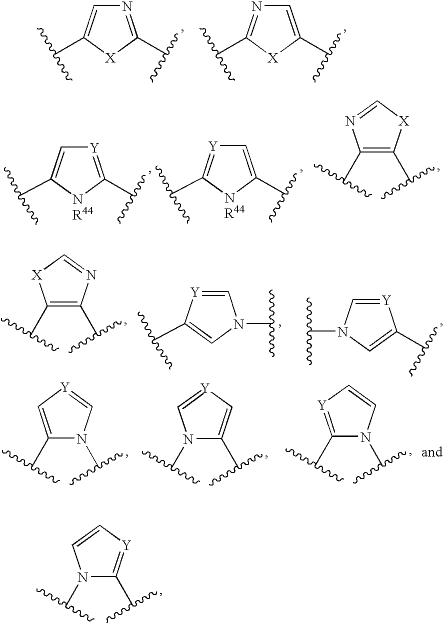

wherein X is O, S, N(H), or N(C₁-C₆ alkyl), Y is O, S, or N, and R⁴⁴ is H or C₁-C₆ alkyl.

Also preferred is V which is a 5-membered heteroarylenyl diradicals selected from:

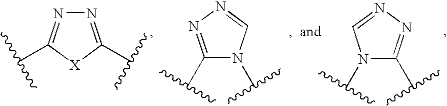

wherein X is O, S, N(H), or N(C₁-C₆ alkyl).

Also preferred is V which is a 5-membered heteroarylenyl diradicals selected from:

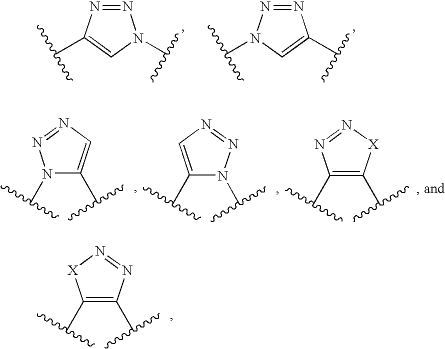

wherein X is O, S, N(H), or N(C₁-C₆ alkyl).

Also preferred is V which is a 5-membered heteroarylenyl diradicals selected from:

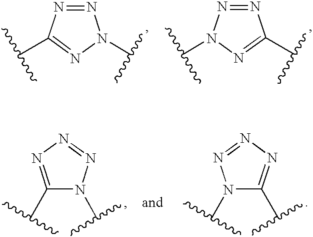

It should be appreciated that a 3- to 7-membered heterocycloalkyl or an 8- to 10-membered heterobicycloalkyl includes 3-azabicyclo[3.1.0]hexanyl, 3-azabicyclo[4.1.0]-heptanyl, azetidinyl, dihydrofuranyl, dihydropyranyl, dihydrothienyl, dioxanyl, 1,3-dioxolanyl, 1,4-dithianyl, hexahydroazepinyl, hexahydropyrimidine, imidazolidinyl, imidazolinyl, isoxazolidinyl, morpholinyl, oxazolidinyl, piperazinyl, piperidinyl, 2H-pyranyl, 4H-pyranyl, pyrazolidinyl, pyrazolinyl, pyrrolidinyl, 2-pyrrolinyl, 3-pyrrolinyl, quinolizinyl, tetrahydrofuranyl, tetrahydropyranyl, 1,2,3,6-tetrahydropyridinyl, tetrahydrothienyl, tetrahydrothiopyranyl, thiomorpholinyl, thioxanyl, and trithianyl. The foregoing groups, as derived from the compounds listed above, can be C-attached or N-attached where such is possible. For example, a group derived from piperidine can be piperidin-1-yl (N-attached) or piperidin-4-yl (C-attached).

It should be appreciated that tautomeric forms (i.e., oxo forms) of substituted 5- or 6-membered heteroaryl or an 8- to 10-membered heterobiaryl groups bearing a hydroxy substituent on a carbon atom are included in the present invention.

It should be appreciated that the invention compounds further comprise compounds of Formula I wherein an indanyl, pentalenyl, indenyl, azulenyl, fluorenyl, or tetrahydronaphthyl group has been inserted in place of a phenyl or naphthyl group defined above for G¹ and G² in Formula I.

It should be appreciated that the invention compounds further comprise compounds of Formula I which are substituted with from 1 to 6 substituents, wherein the substituent(s) is selected from a group containing every chemically and pharmaceutically suitable substituent.

The phrase “chemically and pharmaceutically suitable substituent” is intended to mean a chemically and pharmaceutically acceptable functional group or moiety that does not negate the inhibitory activity of the inventive compounds or impart a degree of toxicity that would make the resulting substituted compound unsuitable for use as a pharmaceutical or veterinary agent. Such suitable substituents include those recited above for Formula I and those which may be routinely selected by those skilled in the art. Illustrative examples of suitable substituents include, but are not limited to halo groups, perfluoroalkyl groups, perfluoroalkoxy groups, alkyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, carboxy groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, an arylsulfonyl groups and the like.

The term “(E)” means entgegen, and designates that the conformation about the double bond to which the term refers is the conformation having the two higher-ranking substituent groups, as determined according to the Cahn-Ingold-Prelog ranking system, on opposite sides of the double bond. An (E) double bond is illustrated below by the compound of Formula (W)

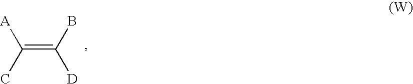

wherein the two higher-ranking substituents are groups A and D.

The term “(Z)” means zusammen, and designates that the conformation about the double bond to which the term refers is the conformation having the two higher-ranking substituent groups, as determined according to the Cahn-Ingold-Prelog ranking system, on the same side of the double bond. A (Z) double bond is illustrated below by the compound of Formula (X)

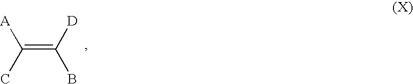

wherein the two higher-ranking substituents are groups A and D.

It should be appreciated that the symbol “ [symbol] ” in front of a bond from a structure indicates that the bond is a radical point of attachment in the structure.

In a compound of Formula I, or a pharmaceutically acceptable salt thereof, it should be appreciated that in any (C₁-C₆ alkyl)₂-N group, the C₁-C₆ alkyl groups may be optionally taken together with the nitrogen atom to which they are attached to form a 5- or 6-membered heterocycloalkyl.

It should be appreciated that each group and each substituent recited above is independently selected unless otherwise indicated.

For the purposes of this invention, the term “arthritis”, which is synonymous with the phrase “arthritic condition”, includes osteoarthritis, inflammatory erosive osteoarthritis, rheumatoid arthritis, degenerative joint disease, spondyloarthropathies, gouty arthritis, systemic lupus erythematosus, juvenile arthritis, and psoriatic arthritis. An inhibitor of MMP-13 having an anti-arthritic effect is a compound as defined above that inhibits the progress, prevents further progress, or reverses progression, in part or in whole, of any one or more symptoms of any one of the arthritic diseases and disorders listed above.

It should be appreciated that osteoarthritis is itself a noninflammatory condition that may be present for years in a patient before any symptoms such as pain are appreciated by the patient.

The term “patient” means a mammal. Preferred patients are humans, cats, dogs, cows, horses, pigs, and sheep.

The term “animal” means a mammal that has an MMP-13 enzyme. Preferred animals include humans, cats, dogs, horses, pigs, sheep, cows, monkeys, rats, mice, guinea pigs, and rabbits.

The term “mammal” includes humans, companion animals such as cats and dogs, primates such as monkeys and chimpanzees, and livestock animals such as horses, cows, pigs, and sheep.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

The phrase “pharmaceutical composition” means a composition suitable for administration to a patient in medical or veterinary use. Pharmaceutical compositions may be in solid or liquid forms. Administration is as described below.

The term “admixed” and the phrase “in admixture” are synonymous and mean in a state of being in a homogeneous or heterogeneous mixture. Preferred is a homogeneous mixture.

The phrases “effective amount” and “therapeutically effective amount” are synonymous and mean an amount of a compound of the present invention, a pharmaceutically acceptable salt thereof, sufficient to prevent the condition being prevented, or inhibit the worsening of, or effect an improvement of, the condition being treated, when administered to a patient suffering from a disease that is mediated by an MMP-13.

The phrase “treating”, which is related to the terms “treat” and “treated”, means administration of an invention compound as defined above that inhibits the progress, prevents further progress, or reverses progression, in part or in whole, of any one or more symptoms or pathological hallmarks of any one of the diseases and disorders listed above.

The phrase “MMP-13 inhibiting amount” means an amount of invention compound, or a pharmaceutically acceptable salt thereof, sufficient to inhibit an enzyme matrix metalloproteinase-13, including a truncated form thereof, including a catalytic domain thereof, in a particular animal or animal population. For example in a human or other mammal, an MMP-13 inhibiting amount can be determined experimentally in a laboratory or clinical setting, or may be the amount required by the guidelines of the United States Food and Drug Administration, or equivalent foreign agency, for the particular MMP-13 enzyme and patient being treated.

The phrase “disease mediated by an MMP-13 enzyme” means any mammalian disease or disorder that exhibits a pathology or symptom that is initiated, worsened, or otherwise promoted by a biological activity of an MMP-13 enzyme, either directly or indirectly. A skilled artisan may readily identify a disease mediated by an MMP-13 enzyme by examining the diseased tissue or fluid, or cells contained therein, for the presence of MMP-13, overactivity therefrom, or excess extracellular matrix cleavage products thereby, such as proteoglycan, hydroxyproline, or type II collagen.

The term “IC₅₀” means the concentration of a compound, usually expressed in micromolar or nanomolar, required to inhibit an enzyme's catalytic activity by 50%.

The terms “ED₄₀” and “ED₃₀” mean the concentrations of an invention compound, usually expressed in micromolar or nanomolar, required to treat a disease in about 40% or 30%, respectively, of a patient group.

As used herein, the phrase “cartilage damage” means a disorder of hyaline cartilage, including articular cartilage, and subchondral bone characterized by hypertrophy of tissues in and around the involved joints, which may or may not be accompanied by deterioration of hyaline cartilage surface. Cartilage damage also refers to a MMP-13 mediated disorder of elastic cartilage (e.g., in an ear or epiglottis) or fibrocartilage (e.g., in intervertebral disks).

The phrases “invention compound” and “compound of Formula I, or a pharmaceutically acceptable salt thereof,” include any tautomer thereof, or any other form thereof, as fully defined above. All of the above-describe forms of an invention compound are included by the phrase “invention compound”, a “compound of the present invention,” a “compound of Formula I”, or a “compound of Formula I, or a pharmaceutically acceptable salt thereof”, or any named species thereof, as being part of an invention embodiment unless specifically excluded therefrom.

The phrase “invention combination” means an invention compound as described above, in combination with another therapeutic agent, as described above.

The term “drug”, which is synonymous with the phrases “active component”, “active compound”, and “active ingredient”, includes celecoxib, or a pharmaceutically acceptable salt thereof, valdecoxib, or a pharmaceutically acceptable salt thereof, or an invention compound, and may further include one or two of the other therapeutic agents described above.

The term “nontoxic” when used alone means the efficacious dose is 10 times or greater than the dose at which a toxic effect is observed in 10% or more of a patient population.

It should be appreciated that an invention compound may be administered in an amount which is “sufficiently nontoxic.” This amount may be an efficacious dose which may potentially produce toxic symptoms in certain patients at certain doses, but because of the pernicious nature of the disease being treated and the risk/benefit value to the patient or patient population of the invention compound being used, it is acceptable to medical or veterinary practitioners and drug regulatory authorities to use a sufficiently nontoxic dose. A sufficiently nontoxic dose may be an efficacious dose at which a majority of patients experience toxicity but wherein the disease being treated is a life-threatening disease such as cancer, including breast cancer, and there are no better treatment options. Alternatively, a sufficiently nontoxic dose may be a generally nontoxic efficacious dose at which a certain majority of patients being treated do not experience drug-related toxicity, although a small percentage of the patient population may be susceptible to an idiosyncratic toxic effect at the dose.

It should be appreciated that COX-2 is also known as prostaglandin synthase-2, prostaglandin PGH₂ synthase, and prostaglandin-H₂ synthase-2.

A selective inhibitor of COX-2 means a compound that inhibits COX-2 selectively versus COX-1 such that a ratio of IC₅₀ for a compound with COX-1 divided by a ratio of IC₅₀ for the compound with COX-2 is greater than, or equal to, 5, where the ratios are determined in one or more assays. All that is required to determine whether a compound is a selective COX-2 inhibitor is to assay a compound in one of a number of well know assays in the art.

The term “NSAID” is an acronym for the phrase “nonsteroidal anti-inflammatory drug”, which means any compound which inhibits cyclooxygenase-1 (“COX-1”) and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetominophen, indomethacin, and phenylbutazone. Selective inhibitors of cyclooxygenase-2 as described above may be considered to be NSAIDs also.

The phrase “tertiary organic amine” means a trisubstituted nitrogen group wherein the 3 substituents are independently selected from C₁-C₆ alkyl, C₃-C₆ cycloalkyl, benzyl, or wherein two of the substituents are taken together with the nitrogen atom to which they are bonded to form a 5- or 6-membered, monocyclic heterocycle containing one nitrogen atom and carbon atoms, and the third substituent is selected from C₁-C₆ alkyl and benzyl, or wherein the three substituents are taken together with the nitrogen atom to which they are bonded to form a 7- to 12-membered bicyclic heterocycle containing 1 or 2 nitrogen atoms and carbon atoms, and optionally a C═N double bond when 2 nitrogen atoms are present. Illustrative examples of tertiary organic amine include triethylamine, diisopropylethylamine, benzyl diethylamino, dicyclohexylmethyl-amine, 1,8-diazabicycle[5.4.0]undec-7-ene (DBU), 1,4-diazabicyclo[2.2.2]octane (TED), and 1,5-diazabicycle[4.3.0]non-5-ene.

It should be appreciated that the matrix metalloproteinases include, but are not limited to, the following enzymes:

MMP-1, also known as interstitial collagenase, collagenase-1, or fibroblast-type collagenase;

MMP-2, also known as gelatinase A or 72 kDa Type IV collagenase;

MMP-3, also known as stromelysin or stromelysin-1;

MMP-7, also known as matrilysin or PUMP-1;

MMP-8, also known as collagenase-2, neutrophil collagenase or polymorphonuclear-type (“PMN-type”) collagenase;

MMP-9, also known as gelatinase B or 92 kDa Type IV collagenase;

MMP-10, also known as stromelysin-2;

MMP-11, also known as stromelysin-3;

MMP-12, also known as macrophage metalloelastase;

MMP-13, also known as collagenase-3;

MMP-14, also known as membrane-type (“MT”) 1-MMP or MT1-MMP;

MMP-15, also known as MT2-MMP;

MMP-16, also known as MT3-MMP;

MMP-17, also known as MT4-MMP;

MMP-18, also known as collagenase-4;

MMP-19, also known as RASI-1 and RASI-6;

MMP-20, also known as enamelysin;

MMP-23, also referred to as “MMP-21” in reproductive tissues;

MMP-24, also known as MT5-MMP;

MMP-25, also known as MT6-MMP and leukolysin;

MMP-26, also known as matrilysin-2 and endometase;

MMP-27; and

MMP-28, also known as epilysin.

It should be appreciated that the S1′ site of MMP-13 was previously thought to be a grossly linear channel which contained an opening at the top that allowed an amino acid side chain from a substrate molecule to enter during binding, and was closed at the bottom. It has been discovered that the S1′ site is actually composed of an S1′ channel angularly connected to a newly discovered pocket which applicant calls the S1″ site. The S1″ site is open to solvent at the bottom, which can expose a functional group of the invention compounds to solvent.

For illustrative purposes, the S1′ site of the MMP-13 enzyme can now be thought of as being like a sock with a hole in the toes, wherein the S1′ channel is the region from approximately the opening to the ankle area of the sock, and the S1′ site is the foot region below the ankle, which foot region is angularly connected to the ankle region. However, the invention compounds do not necessarily bind in the S1′ site of MMP-13.

More particularly, the S1′ channel is a specific part of the S1′ site and is formed largely by Leu218 (leucine 218 of an MMP-13 enzyme), Val219 (valine 219 of an MMP-13 enzyme), His222 (histidine 222 of an MMP-13 enzyme) and by residues from Leu239 (leucine 239 of an MMP-13 enzyme) to Tyr244 (tyrosine 244 of an MMP-13 enzyme). The S1″ binding site which has been newly discovered is defined by residues from Tyr246 (tyrosine 246 of an MMP-13 enzyme)) to Pro255 (proline 255 of an MMP-13 enzyme). The S1″ site contains at least two hydrogen bond donors and aromatic groups which interact with an invention compound.

Without wishing to be bound by any particular theory, the inventors believe that the S1″ site could be a recognition site for triple helix collagen, a natural substrate for MMP-13. It is possible that the conformation of the S1″ site is modified only when an appropriate compound binds to MMP-13, thereby interfering with the collagen recognition process. This newly discovered pattern of binding offers the possibility of greater selectivity than what is achievable with the binding pattern of known selective inhibitors of MMP-13, wherein the known binding pattern requires ligation of the catalytic zinc atom at the active site and occupation the S1′ channel, but not the S1″ site. Alternatively, inhibition of the MMP may result from a suitable electronic interaction (e.g., hydrogen bonding) between an invention compound and one or more of the histidine residues that ligate the catalytic zinc of MMP-13.

The compounds of Formula I, or pharmaceutically acceptable salts thereof, are believed to be allosteric inhibitors of MMP-13. An invention compound which is an allosteric inhibitor of MMP-13 is any compound of Formula I that binds allosterically into the S1′ site of the MMP-13 enzyme, including the S1′ channel, and a newly discovered S1″ site, and ligates, coordinates, or binds the catalytic zinc of the MMP-13 with a group Z, wherein Z is as defined above.

Advantages:

The advantages of using an invention compound in a method of the instant invention include, but are not limited to, the sufficiently nontoxic nature of the compounds at and substantially above therapeutically effective doses, their ease of preparation, the fact that the compounds are well-tolerated, and the ease of administration of the drugs to a patient.

The invention compounds that are selective, highly selective, or specific inhibitors of MMP-13 have a further advantage: The invention compounds can target an MMP-13 mediated disease with fewer side effects than MMP-13 inhibitor compounds which also inhibit other MMP enzymes. The invention compounds are thus particularly advantageous over other MMP-13 inhibitors that also have IC₅₀'s of less than or equal to 1 μM, increasingly preferably 500 nM, 250 nM, 100 nM, and 50 nM, with one, increasingly preferably two, three, four, five, or more additional MMP enzymes selected from: MMP-1, MMP-2, MMP-3, MMP-7, MMP-8, MMP-9, MMP-12, MMP-14, and MMP-17. Without wishing to be bound by a theory, it seems that the instant invention compounds are allosteric inhibitors of MMP-13 in that they bind at a different location from where a natural substrate binds to MMP-13.

It should be appreciated that virtually all MMP inhibitors tested clinically to date have been nonselective MMP inhibitors that bind to multiple MMPs by coordinating to the catalytic zinc cation at the substrate binding site of the MMPs. These nonselective MMP inhibitors typically have exhibited an undesirable side effect known as muscoloskeletal syndrome (“MSS”). The side effect MSS is associated with administering an inhibitor of multiple MMP enzymes or an inhibitor of a particular MMP enzyme such as possibly MMP-1. MSS will be significantly reduced in type and severity by administering an invention compound instead of a nonselective MMP inhibitor.

The invention compounds are thus superior to compounds that interact with the catalytic zinc cation of the MMP-13 enzyme, even if such compounds show some selectivity for the MMP-13 over other MMPs.

The MMP selectivity advantage of the instant compounds will also significantly increase the likelihood that agencies which regulate new drug approvals, such as the United States Food and Drug Administration (“FDA”), will approve the instant compounds versus a competing similar compound that does not allosterically bind to MMP-13 as discussed above even in the unlikely event that the two compounds behaved similarly in clinical trials. These regulatory agencies are increasingly aware that clinical trials, which test drug in limited population groups, do not always uncover safety problems with a drug, and, all other things being equal, the agencies thus favor the most selective drug.

Another important advantage is that the cartilage damage inhibiting properties of the invention compounds provide patients suffering from osteoarthritis a means of inhibiting, or even reversing, the underlying disease pathology of cartilage degradation. There is no currently FDA-approved drug for disease modification of the cartilage damage of osteoarthritis. Osteoarthritis (“OA”) patients given an invention compound may experience improved OA signs and symptoms such as improved joint function or a reduction of joint stiffness, pain, or inflammation, or a combination of the same.

Preparation of Compounds:

An invention compound that is an allosteric inhibitor of MMP-13 may be readily synthesized by one of ordinary skill in the medicinal or organic chemistry arts according to the procedures outlined in the Schemes and Compound Examples below.

Any invention compound is readily available, either commercially, or by synthetic methodology, well known to those skilled in the art of organic chemistry. For specific syntheses, see the examples below and the preparations of invention compound outlined in the Schemes below.

Invention compounds, and intermediates for the syntheses thereof, may be prepared by one of ordinary skill in the art of organic chemistry by adapting various synthetic procedures incorporated by reference above or that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods,1989,by Wiley-Interscience; the text Advanced Organic Chemistry, 4^{th} edition, by Jerry March, Wiley-Interscience, New York,1992; or the Handbook of Heterocyclic Chemistry by Alan R. Katritzky, Pergamon Press Ltd, London, 1985, to name a few. More recent editions of some of the above references may be available.

Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the invention compounds may use starting materials, atmospheres, reagents, solvents, and catalysts that may be purchased from commercial sources or they may be readily prepared by adapting procedures in the references or resources cited above. Commercial sources of starting materials, reagents, solvents, and catalysts useful in preparing invention compounds and intermediates in the syntheses thereof include, for example, The Aldrich Chemical Company, and other subsidiaries of Sigma-Aldrich Corporation, St. Louis, Mo., BACHEM, BACHEM A.G., Switzerland, and Lancaster Synthesis Ltd, United Kingdom.

Syntheses of some invention compounds may utilize starting materials, intermediates, or reaction products that contain a reactive functional group. During chemical reactions, a reactive functional group may be protected from reacting by a protecting group that renders the reactive functional group substantially inert to the reaction conditions employed. A protecting group is introduced onto a starting material prior to carrying out the reaction step for which a protecting group is needed. Once the protecting group is no longer needed, the protecting group can be removed by a conventional method.

It is well within the ordinary skill in the art to introduce protecting groups during a synthesis of a compound of Formula I, or a pharmaceutically acceptable salt thereof, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: New York, 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

More particularly, compounds of Formula I may be prepared according to the synthetic route outlined below in Scheme 1.

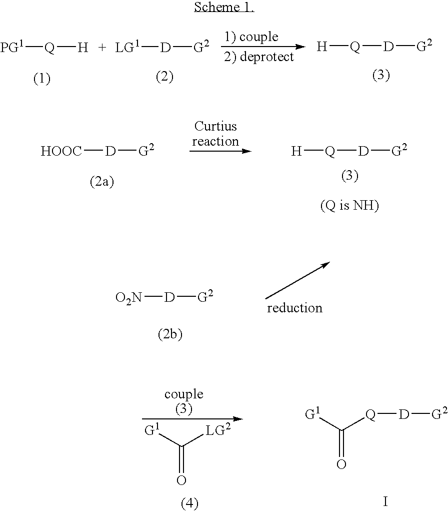

In Scheme 1, a compound of formula (1), wherein Q is O, N(H), or N(C₁-C₆ alkyl), and PG¹ is a suitable alcohol or amine protecting group, is allowed to react with a compound of formula (2), wherein LG¹ is a leaving group selected from Cl, Br, I, and CF₃SO₃, and G² and D are as defined above for Formula I, in the presence of a suitable coupling reagent such as copper bronze, a palladium catalyst, including bis(triphenylphosphinyl) palladium chloride palladium tetrakis triphenylphosphine, palladium acetate, or palladium chloride, in the presence of a base such as a tertiary organic amine, including triethylamine or diisopropylethylamine, or potassium acetate in a suitable aprotic solvent such as tetrahydrofuran (“THF”), heptane, or ethyl acetate, followed by deprotection by removal of PG¹ to yield a compound of formula (3). This coupling has been developed by Professor Buchwald and others. The general coupling reaction works for a variety of D groups, including aryl or heteroaryl D groups, under a variety of conditions. See Comprehensive Organic Transformations, by Richard C. Larock, VCH Publishers, Inc, New York, 1989:397-400 and references cited therein; and Advanced Organic Chemistry by Jerry March, John Wiley & Sons, New York, 4^{th} edition, 1992:717-718, and references cited therein.

Alternatively, the compound of formula (3) wherein Q is NH may be prepared from the corresponding carboxylic acid of formula (2a) via a conventional Curtius rearrangement or by reduction of the corresponding nitro compound of formula (2b).

Alternatively, the compound of formula (3) wherein Q is NH or OH may be purchased from commercial sources.

Alternatively, the compound of formula (3) wherein Q is OH may be prepared from the corresponding des-hydroxy compound by conventional aryl or heteroaryl oxidations using, for example, hydrogen peroxide or trifluoroperacetic acid and a suitable acid such as aluminum trichloride or hydrofluoric acid, or by conventional conversion of the corresponding halo compound via a suitable organo lithium or Grignard intermediate and an oxidant such as, for example, meta-chloroperbenzoic acid (“mCPBA”).

The compound of formula (3) is then allowed to react with a carboxylic acid or derivative thereof of formula (4) such as the corresponding acid halide (especially chloride), anhydride with acetic acid or trifluoroacetic acid, or activated derivative thereof formed by reaction with dicyclohexylcarbodiimide (“DCC”), water soluble versions thereof, N,N′-carbonyldiimidazole (“CDI”), and the like to give a compound of Formula I.

Illustrative syntheses of specific invention compounds are described below in the Compound Examples.

### COMPOUND EXAMPLES

### Compound Example A1

4-{6-[2-(3-Methoxy-phenyl)-acetylamino]-4-oxo-4H-quinazolin-3-ylmethyl}-benzoic Acid

This compound was synthesized as described below in Compound Example B2 using 4-(6-iodo-4-oxo-4H-quinazolin-3-yl)benzoic acid tert-butyl ester and 3-methoxyphenylacetic acid in place of 4-(7-bromo-1-oxo-1H-isoquinolin-2-ylmethyl)-benzoic acid tert-butyl ester and 4-methoxyphenyl-acetic acid, respectively.

¹H-NMR (DMSO); 12.91 (s, 1H), 10.51 (s, 1H), 8.44 (s, 2H), 7.98-7.96 (d, 1H), 7.90-7.88 (d, 2H), 7.66-7.64 (s, 1H), 7.41-7.39 (d, 2H), 7.24-7.19 (t, 1H), 6.90-6.88 (m, 2H), 6.81-6.79 (d, 1H), 5.24 (s, 2H), 3.72 (s, 2H)

MS: M⁺+1=444.1 Da

### Compound Example A2

4-{7-Fluoro-6-[2-(3-methoxy-phenyl)-acetylamino]-4-oxo-4H-quinazolin-3-ylmethyl}-benzoic Acid

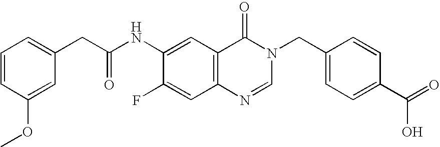

Step (1): Preparation of 4-(7-fluoro-6-nitro-4-oxo-4H-quinazolin-3-ylmethyl)benzoic Acid Tert-butyl Ester

A solution of 7-fluoro-6-nitro-3H-quinazolin-4-one (4.00 g, 19.1 mmol) in DMF (60 mL) was treated with CsCO₃ (8.10 g, 24.9 mmol), then stirred at room temperature for 30 minutes. To the solution was added 4-bromomethylbenzoic acid tert-butyl ester (6.72 g, 24.9 mmol), and the reaction mixture stirred at room temperature for 2 hours, then overnight at 60° C. The DMF was removed by evaporation, the residue dissolved in ethyl acetate (“EtOAc”), washed with 1N HCl, brine, dried over MgSO₄, and evaporated onto silica gel. The silica gel was eluted on a 4.5×20 cm silica gel column with hexanes/EtOAc 1:1. The appropriate fractions were combined and dried to give 2.44 g (31.9%) of 4-(7-fluoro-6-nitro-4-oxo-4H-quinazolin-3-ylmethyl)benzoic acid tert-butyl ester as a yellow solid.

¹H-NMR (CDCl₃); 9.04-9.02 (d, 1H), 8.18 (s, 1H), 8.00-7.97 (d, 2H), 7.55-7.53 (d, 1H), 7.40-7.38 (d, 2H), 5.24 (s, 2H), 1.58 (s, 9H)

MS: M⁺+1=400.0 Da

Step (2): Preparation of 4-(6-amino-7-fluoro-4-oxo-4H-quinazolin-3-ylmethyl)benzoic Acid Tert-butyl Ester

A solution of 4-(7-fluoro-6-nitro-4-oxo-4H-quinazolin-3-ylmethyl)benzoic acid tert-butyl ester (1.06 g, 2.65 mmol, Step (1)) in THF (50 mL) was treated with Raney nickel (“RaNi”) (0.5 g) and hydrogenated under 50 psi of H₂ for 19 hours. The solution was filtered, evaporated onto silica gel, and the silica gel eluted on a 3.5×17 cm silica gel column with hexanes/EtOAc 4:1. The appropriate fractions were combined, evaporated, and dried to give 0.50 g (47.0%) of 4-(6-amino-7-fluoro-4-oxo-4H-quinazolin-3-ylmethyl)benzoic acid tert-butyl ester.

¹H-NMR (CDCl₃); 8.27 (s, 1H), 7.84-7.83 (d, 2H), 7.42-7.37 (m, 3H), 7.33-7.30 (d, 1H), 5.76 (s, 2H), 5.19 (s, 2H), 1.49 (s, 9H)

MS: M⁺+1=370.1 Da

Step (3): Preparation of 4-{6-[2-(3-methoxyphenyl)acetylamino]-4-oxo-4H-quinazolin-3-ylmethyl}benzoic Acid Tert-butyl Ester

A solution of 3-methoxyphenylacetic acid (0.17 g, 1.0 mmol), 1-(3-dimethylaminopropyl)-3-ethyl-carbodiimide hydrochloride (“EDAC.HCl”, also abbreviated as “EDC”, 0.19 g, 1.0 mmol), and HOBT (0.14 g, 1.0 mmol) in DMF (5 mL) was stirred at room temperature for 30 minutes. To this mixture was added 4-(6-amino-7-fluoro-4-oxo-4H-quinazolin-3-ylmethyl)benzoic acid tert-butyl ester (0.25 g, 0.68 mmol, Step (2)), and the reaction mixture stirred for 2 days at room temperature, affording predominately starting material. The reaction mixture was then heated at 80° C. for 2 days, cooled to room temperature, treated with water (3 mL), saturated aqueous NaHCO₃ (3 mL), then water (3 mL), and the mixture stirred at room temperature for 30 minutes. The mixture was diluted with EtOAc, washed with water, brine, dried over MgSO₄, and evaporated onto silica gel. The silica gel was eluted on a 2.5×18 cm silica gel column with EtOAc/hexanes 2:1. The appropriate fractions were combined, evaporated, and dried to give impure 4-{6-[2-(3-methoxyphenyl)acetylamino]-4-oxo-4H-quinazolin-3-ylmethyl}benzoic acid tert-butyl ester, which was used directly in the next reaction.

Step (4): Preparation of 4-{7-fluoro-6-[2-(3-methoxy-phenyl)-acetylamino]-4-oxo-4H-quinazolin-3-ylmethyl}-benzoic Acid

The impure 4-{6-[2-(3-methoxyphenyl)acetylamino]-4-oxo-4H-quinazolin-3-ylmethyl}benzoic acid tert-butyl ester from Step (3) was treated with TFA (6 mL), then stirred at room temperature for 50 minutes. The TFA was evaporated, the resulting solid triturated with EtOAc, collected by filtration, washed with water, followed by EtOAc. Drying afforded 0.0372 g of 4-{7-fluoro-6-[2-(3-methoxy-phenyl)-acetylamino]-4-oxo-4H-quinazolin-3-ylmethyl}-benzoic acid.

¹H-NMR (DMSO); 12.91 (s, 1H), 10.20 (s, 1H), 8.76-8.74 (d, 1H), 8.54 (s, 1H), 7.89-7.86 (d, 2H), 7.60-7.56 (d, 1H), 7.42-7.40 (d, 2H), 7.24-7.20 (t, H), 6.91-6.89 (m, 2H), 6.81-6.79 (d, 1H), 5.23 (s, 2H), 3.72 (s, 2H), 3.71 (s, 3H)

MS: M⁺+1=462.1 Da

### Compound Example A3

4-{6-[2-(4-Methoxy-phenyl)-acetylamino]-4-oxo-4H-quinazolin-3-ylmethyl}-benzoic Acid

The compound was synthesized as described below in Compound Example B2 using 4-(6-iodo-4-oxo-4H-quinazolin-3-yl)benzoic acid tert-butyl ester in place of 4-(7-bromo-1-oxo-1H-isoquinolin-2-ylmethyl)-benzoic acid tert-butyl ester.

¹H-NMR (DMSO); 12.91 (s, 1H), 10.48 (s, 1H), 8.47 (s, 2H), 7.99-7.95 (d, 1H), 7.89-7.86 (d, 2H), 7.67-7.63 (d, 1H), 7.41-7.39 (d, 2H), 7.24-7.19 (t, 1H), 6.92-6.89 (m, 2H), 6.81-6.78 (d, 1H), 5.25 (s, 2H), 3.72 (s, 2H)

MS: M⁺+1=444.1 Da

### Compound Example A4

4-{6-[2-(4-Fluoro-phenyl)-acetylamino]-4-oxo-4H-quinazolin-3-ylmethyl}-benzoic Acid

This compound was synthesized as described below in Compound Example B2 using 4-(6-iodo-4-oxo-4H-quinazolin-3-yl)benzoic acid tert-butyl ester and 4-fluorophenylacetic acid in place of 4-(7-bromo-1-oxo-1H-isoquinolin-2-ylmethyl)-benzoic acid tert-butyl ester and 4-methoxyphenyl-acetic acid, respectively.

¹H-NMR (DMSO); 12.91 (bs, 1H), 10.51 (s, 1H), 8.45 (m, 3H), 7.98-7.96 (d, 1H), 7.90-7.88 (d, 2H), 7.67-7.63 (d, 1H), 7.41-7.33 (m, 4H), 7.15-7.12 (m, 2H), 5.24 (s, 2H), 3.65 (s, 2H)

MS: M⁺+1=432.1 Da

### Compound Example A5

4-{6-[2-(3-Fluoro-phenyl)-acetylamino]-4-oxo-4H-quinazolin-3-ylmethyl}-benzoic Acid

This compound was synthesized as described below in Compound Example B2 using 4-(6-iodo-4-oxo-4H-quinazolin-3-yl)benzoic acid tert-butyl ester and 3-fluorophenylacetic acid in place of 4-(7-bromo-1-oxo-1H-isoquinolin-2-ylmethyl)-benzoic acid tert-butyl ester and 4-methoxypheny-lacetic acid, respectively.

¹H-NMR (DMSO); 12.91 (s, 1H), 10.56 (s, 1H), 8.44 (s, 2H), 7.98-7.96 (d, 1H), 7.90-7.87 (d, 2H), 7.67-7.64 (d, 1H), 7.42-7.33 (m, 3H), 7.18-7.14 (d, 2H), 7.08-7.04 (t, 1H), 5.23 (s, 2H), 3.71 (s, 2H)

MS: M⁺+1=432.1 Da

### Compound Example A6

4-{7-fluoro-6-[2-(4-methoxyphenyl)acetylamino]-4-oxo-4H-quinazolin-3-ylmethyl}benzoic Acid

The title compound was synthesized in a manner analogous to that previously described in Compound Example A2 using 4-(6-amino-7-fluoro-4-oxo-4H-quinazolin-3-ylmethyl)benzoic acid tert-butyl ester and 4-methoxyphenylacetic acid in place of 3-methoxyphenylacetic acid.

¹H-NMR (DMSO); 12.91 (s, 1H), 10.16 (s, 1H), 8.75-8.73 (d, 1H), 8.53 (s, 1H), 7.90-7.87 (d, 2H), 7.59-7.56 (d, 1H), 7.42-7.40 (d, 2H), 7.26-7.23 (d, 2H), 6.89-6.86 (d, 2H), 5.24 (s, 2H), 3.71 (s, 3H), 3.70 (s, 2H)

MS: M⁺+1=462.1 Da

### Compound Example A7

4-[7-fluoro-4-oxo-6-(2-p-tolylacetylamino)-4H-quinazolin-3-ylmethyl]benzoic Acid

The title compound was synthesized in a manner analogous to that previously described in Compound Example A2 using 4-(6-amino-7-fluoro-4-oxo-4H-quinazolin-3-ylmethyl)benzoic acid tert-butyl ester and p-tolylacetic acid in place of 3-methoxyphenylacetic acid.

¹H-NMR (DMSO); 12.91 (bs, 1H), 10.19 (s, 1H), 8.74-8.72 (d, 1H), 8.53 (s, 1H), 7.89-7.86 (d, 2H), 7.59-7.56 (d, 1H), 7.42-7.39 (d, 2H), 7.22-7.19 (d, 2H), 7.12-7.10 (d, 2H), 5.23 (s, 2H), 3.71 (s, 2H), 2.25 (s, 3H)

MS: M⁺+1=446.1 Da

### Compound Example A8

4-{7-fluoro-6-[2-(4-hydroxyphenyl)acetylamino]-4-oxo-4H-quinazolin-3-ylmethyl}benzoic Acid

The title compound was synthesized in a manner analogous to that previously described in Compound Example A2 using 4-(6-amino-7-fluoro-4-oxo-4H-quinazolin-3-ylmethyl)benzoic acid tert-butyl ester and 4-hydroxyphenylacetic acid in place of 3-methoxyphenylacetic acid.

¹H-NMR (DMSO); 12.91 (s, 1H), 10.11 (s, 1H), 9.23 (s, 1H), 8.76-8.73 (d, 1H), 8.52 (s, 1H), 7.89-7.87 (d, 2H), 7.59-7.55 (d, 1H), 7.42-7.40 (d, 2H), 7.12-7.11 (d, 2H), 6.69-6.67 (d, 2H), 5.23 (s, 2H), 3.63 (s, 2H)

MS: M⁺+1=448.0 Da

### Compound Example B1

4-{7-[2-(3-Methoxy-phenyl)-acetylamino]-1-oxo-1H-isoquinolin-2-ylmethyl}-benzoic Acid

The title compound was synthesized as described below in Compound Example B2 using 3-methoxyphenylacetic acid in place of 4-methoxyphenylacetic acid.

¹H-NMR (DMSO); 12.91 (s, 1H), 10.43 (s, 1H), 8.49 (s, 1H), 7.94-7.87 (m, 3H), 7.62-7.59 (d, 1H), 7.46-7.44 (d, 1H), 7.37-7.34 (d, 2H), 7.23-7.20 (t, 1H), 6.92-6.89 (m, 2H), 6.82-6.80 (d, 1H), 6.61-6.58 (d, 1H), 5.22 (s, 2H), 3.73 (s, 3H), 3.62 (s, 2H).

MS: M⁺+1=443.1 Da

### Compound Example B2

4-{7-[2-(4-Methoxy-phenyl)-acetylamino]-1-oxo-1H-isoquinolin-2-ylmethyl}-benzoic Acid

Step (1) Preparation of 4-(7-amino-1-oxo-1H-isoquinolin-2-ylmethyl)benzoic acid tert-butyl ester

Into a sealed reactor was placed 4-(7-bromo-1-oxo-1H-isoquinolin-2-ylmethyl)benzoic acid tert-butyl ester (6.40 g, 15.4 mmol), copper bronze (0.1 g), and liquid NH₃ (80 mL). The reactor was heated to 70° C. for 62 hours, cooled to room temperature, filtered through Celite, and washed with tetrahydrofuran (“THF”). The filtrate was evaporated, the residue dissolved in EtOAc, and evaporated onto silica gel. The silica gel eluted on a 3.5×18 cm silica gel column with ethyl acetate/hexanes 2:1. Evaporation of the appropriate fractions afforded a solid that was triturated with ether, collected, and dried to give 3.69 g (68.2%) of 4-(7-amino-1-oxo-1H-isoquinolin-2-ylmethyl)benzoic acid tert-butyl ester.

¹H-NMR (CDCl₃); 7.94-7.91 (d, 2H), 7.68-7.67 (d, 1H), 7.34-7.31 (m, 3H), 7.03-7.00 (d, 1H), 6.84-6.82 (d, 1H), 6.41-6.39 (d, 1H), 5.23 (s, 2H), 1.56 (s, 9H)

MS: M⁺+1=351.1 Da

Step (2): Preparation of 4-{7-[2-(4-methoxyphenyl)acetylamino]-1-oxo-1H-isoquinolin-2-ylmethyl}benzoic Acid Tert-butyl Ester

A solution of 4-methoxyphenylacetic acid (0.20 g, 1.20 mmol), EDAC.HCl (0.23 g, 1.20 mmol), and 1-hydroxybenzotriazole (“HOBT”, 0.16 g, 1.20 mmol) in dimethylformamide (“DMF”, 5 mL) was stirred at room temperature for 30 minutes. To this was added 4-(7-amino-1-oxo-1H-isoquinolin-2-ylmethyl)benzoic acid tert-butyl ester (0.30 g, 0.86 mmol, Step (1)) and the reaction mixture heated to 100° C. for 2 days. The reaction was cooled to room temperature, treated with water (2 mL), saturated aqueous NaHCO₃ (2 mL), then water (2 mL), and the mixture stirred at room temperature for 1 hour. The precipitated solid was collected by filtration, washed with water and dried. The solid was then triturated with hot hexanes/EtOAc 1:1, cooled to room temperature, and collected. Washing with hexanes/EtOAc and drying afforded 0.27 g (62.1%) of 4-{7-[2-(4-methoxyphenyl)acetylamino]-1-oxo-1H-isoquinolin-2-ylmethyl}benzoic acid tert-butyl ester.

¹H-NMR (DMSO); 10.39 (s, 1H), 8.47 (s, 1H), 7.93-7.92 (d, 1H), 7.84-7.82 (d, 2H), 7.57-7.59 (d, 1H), 7.44-7.42 (d, 1H), 7.37-7.35 (d, 2H), 7.25-7.24 (d, 2H), 6.89-6.86 (d, 2H), 6.60-6.59 (d, 1H), 5.23 (s, 2H), 3.70 (s, 3H), 3.57 (s, 2H), 1.50 (s, 9H).

MS: M⁺+1=499.2 Da

Step (3): Preparation of 4-{7-[2-(4-methoxyphenyl)acetylamino]-1-oxo-1H-isoquinolin-2-ylmethyl}benzoic Acid

The product of Step (2), namely 4-{7-[2-(4-methoxyphenyl)acetylamino-1-oxo-1H-isoquinolin-2-ylmethyl}benzoic acid tert-butyl ester, (0.18 g, 0.36 mmol), was treated with trifluoroacetic acid (“TFA”, 6 mL), then stirred at room temperature for 50 minutes. The TFA was evaporated, and the resulting solid triturated with hexanes/ethyl acetate (1:1), collected by filtration, washed with water, then hexanes/ethyl acetate (1:1). Drying afforded 0.14 g (89.5%) of 4-{7-[2-(4-methoxyphenyl)acetylamino]-1-oxo-1H-isoquinolin-2-ylmethyl} benzoic acid.

¹H-NMR (DMSO); 12.88 (s, 1H), 10.36 (s, 1H), 8.47 (s, 1H), 7.93-7.86 (m, 3H), 7.60-7.59 (d, 1H), 7.46-7.43 (d, 1H), 7.36-7.34 (2H), 7.26-7.23 (d, 2H), 6.87-6.85 (d, 2H), 6.61-6.59 (d, 1H), 5.23 (s, 2H), 3.70 (s, 3H), 3.56 (s, 2H)

MS: M⁺+1=443.1 Da

### Compound Example B3

4-{7-[2-(3-Fluoro-phenyl)-acetylamino]-1-oxo-1H-isoquinolin-2-ylmethyl}-benzoic Acid

The title compound was synthesized as described above in Compound Example B2 using 3-fluorophenylacetic acid in place of 4-methoxyphenylacetic acid.

¹H-NMR (DMSO); 12.88 (s, 1H), 10.46 (s, 1H), 8.47 (s, 1H), 9.93-7.84 (m, 3H), 7.62-7.60 (d, 1H), 7.45-7.44 (d, 1H), 7.36-7.33 (m, 3H), 7.18-7.15 (d, 2H), 7.09-7.04 (t, 1H), 6.61-6.59 (d, 1H), 5.22 (s, 2H), 3.69 (s, 2H)

MS: M⁺+1=431.1 Da

### Compound Example B4

4-{7-[2-(4-Fluoro-phenyl)-acetylamino]-1-oxo-1H-isoquinolin-2-ylmethyl}-benzoic Acid

The title compound was synthesized as described above in Compound Example B2 using 4-fluorophenylacetic acid in place of 4-methoxyphenylacetic acid.

¹H-NMR (DMSO); 12.91 (s, 1H), 10.46 (s, 1H), 8.47 (s, 1H), 7.94-7.86 (m, 3H), 7.62-7.58 (d, 1H), 7.46-7.43 (d, 1H), 7.38-7.33 (m, 4H), 7.16-7.10 (t, 2H), 6.62-6.60 (d, 1H), 5.23 (s, 2H), 3.66 (s, 2H)

MS: M⁺+1=431.1 Da

### Evaluating Biological Activity of Invention Compounds

Invention compounds may be tested by one of ordinary skill in the pharmaceutical or medical arts for inhibition of MMP-13 and other MMPs by assaying the test compound as described below in one or more of Biological Methods 1 to 10, and for allosteric inhibition of MMP-13 by assaying the test invention compound for inhibition of MMP-13 in the presence of an inhibitor to the catalytic zinc of MMP-13 as described below in Biological Methods 5 or 6.

Further, an invention compound having an anti-breast cancer, anti-inflammatory, an analgesic, anti-arthritic, or a cartilage damage inhibiting effect, or any combination of these effects, may be readily identified by one of ordinary skill in the pharmaceutical or medical arts by assaying the invention compound in any number of well known assays for measuring determining the invention compound's effects on breast cancer, cartilage damage, arthritis, inflammation, or pain. These assays include in vitro assays that utilize cartilage samples and in vivo assays in whole animals that measure tissue penetration by cancer cells, cartilage degradation, inhibition of inflammation, or pain alleviation.

For example with regard to assaying cartilage damage in vitro, an amount of an invention compound or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content.

Further, in vivo assays to assay cartilage damage may be performed as follows: an amount of an invention compound or control vehicle may be administered with a cartilage damaging agent to an animal, including a human, and the effects of the invention compound being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content.

Several methods of identifying an invention compound with cartilage damage inhibiting properties are described below. The amount to be administered in an assay is dependent upon the particular assay employed, but in any event is not higher than the maximum amount of a compound that the particular assay can effectively accommodate.

Similarly, invention compounds having pain-alleviating properties may be identified using any one of a number of in vivo animal models of pain.

Still similarly, invention compounds having anti-inflammatory properties may be identified using any one of a number of in vivo animal models of inflammation. For example, for an example of inflammation models, see U.S. Pat. No. 6,329,429, which is incorporated herein by reference.

Still similarly, invention compounds having anti-arthritic properties may be identified using any one of a number of in vivo animal models of arthritis. For example, for an example of arthritis models, see also U.S. Pat. No. 6,329,429.

The ability of collagenase inhibitors to inhibit collagenase activity is well known in the art. The degree of inhibition of a particular MMP for a number of compounds has been well documented in the art and those skilled in the art will know how to normalize different assay results to those assays reported herein.

The invention compounds may be assayed for inhibition of MMP-13 or other MMP enzymes according to Biological Methods 1 to 10, and further assaying the test compound for allosteric inhibition of MMP-13 according to Biological Methods 5 or 6, as described below. Except for the assays of Biological Methods 5 and 6, the assays used to evaluate the MMP biological activity of the invention compounds are well-known and routinely used by those skilled in the study of MMP inhibitors and their use to treat clinical conditions. The assays measure the amount by which a test compound reduces the matrix metalloproteinase enzyme-catalyzed hydrolysis of a substrate such as a thiopeptolide or fluorigenic peptide substrate. Such assays are described in detail by Ye et al., in Biochemistry, 1992;31(45):11231-11235, which is incorporated herein by reference.

Some of the particular methods described below use the catalytic domain of the MMP-13 enzyme, namely matrix metalloproteinase-13 catalytic domain (“MMP-13CD”), rather than the corresponding full-length enzyme, MMP-13. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme.

Biological Methods 1 to 10 are described below for illustrative purposes.

1. In Vitro Biological Methods

### Biological Method 1

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain a suitable amount of the MMP enzyme such as an amount described in Ye Qi-Zhuang, et al., 1996, supra, 50 mM of N-2-hydroxyethylpiperazine-N′-2-ethanesulfonic acid buffer (“HEPES,” pH 7.0), 10 mM CaCl₂, 100 μM thiopeptolide substrate, and 1 mM 5,5′-dithio-bis-(2-nitro-benzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Test compounds are evaluated at various concentrations in order to determine their respective IC₅₀ values, the micromolar concentration of compound required to cause a 50% inhibition of catalytic activity of the respective enzyme.

It should be appreciated that the assay buffer used with MMP-3CD was 50 mM N-morpholinoethane sulfonate (“MES”) at pH 6.0 rather than the HEPES buffer at pH 7.0 described above.

Test compounds can be evaluated according to Biological Method 1 at various concentrations in order to determine their respective IC₅₀ values, typically the micromolar concentration of compound required to cause a 50% inhibition of the hydrolytic activity of the respective enzyme.

Some representative compounds of Formula I have been evaluated according to Biological Method 1 for their ability to inhibit MMP-13 and other MMP enzymes, including MMP-1, MMP-2, MMP-3, MMP-7, MMP-8, MMP-9, MMP-12, MMP14, and MMP-17. Inhibitor activity versus other MMPs with the compounds may be determined using, for example, MMP-1FL, which refers to full length interstitial collagenase; MMP-2FL, which refers to full length Gelatinase A; MMP-3CD, which refers to the catalytic domain of stromelysin; MMP-7FL, which refers to full length matrilysin; MMP-9FL, which refers to full length Gelatinase B; MMP-13CD, which refers to the catalytic domain of collagenase 3; and MMP-14CD, which refers to the catalytic domain of MMP-14.

### Biological Method 2

The method of Biological Method 1, except thiopeptolide substrate is replaced with a fluorigenic peptide such as Fluorigenic peptide-1.

### Biological Method 3

Inhibition of MMP-13:

Human recombinant MMP-13 is activated with 2 mM APMA (p-aminophenyl mercuric acetate) for 1.5 hours, at 37° C. and is diluted to 400 mg/mL in assay buffer (50 mM Tris, pH 7.5, 200 mM sodium chloride, 5 mM calcium chloride, 20 μM zinc chloride, 0.02% brij). Twenty-five microliters of diluted enzyme is added per well of a 96 well microfluor plate. The enzyme is then diluted in a 1:4 ratio in the assay by the addition of inhibitor and substrate to give a final concentration in the assay of 100 mg/mL.

10 mM stock solutions of inhibitors are made up in dimethyl sulfoxide and then diluted in assay buffer as per the inhibitor dilution scheme for inhibition of human collagenase (MMP-1): Twenty-five microliters of each concentration is added in triplicate to the microfluor plate. The final concentrations in the assay are 30 μM, 3 μM, 0.3 μM, and 0.03 μM.

Substrate (Dnp-Pro-Cha-Gly-Cys(Me)-His-Ala-Lys(NMA)-NH₂) is prepared as for inhibition of human collagenase (MMP-1) and 50 μL is added to each well to give a final assay concentration of 10 μM. Fluorescence readings (360 nM excitation; 450 emission) are taken at time 0 and every 5 minutes for 1 hour. Positive controls consist of enzyme and substrate with no inhibitor and blanks consist of substrate only.

IC₅₀'s are determined as per inhibition of human collagenase (MMP-1). If IC₅₀'s are reported to be less than 0.03 μM, inhibitors are then assayed at final concentrations of 0.3 μM, 0.03 μM, 0.003 μM and 0.0003 μM.

### Biological Method 4

Collagen Film MMP-13 Assay

Rat type I collagen is radiolabeled with ¹⁴C acetic anhydride (T. E. Cawston and A. J. Barrett, Anal. Biochem., 99, 340-345 (1979)) and used to prepare 96 well plates containing radiolabeled collagen films (Barbara Johnson-Wint, Anal. Biochem., 104, 175-181 (1980)). When a solution containing collagenase is added to the well, the enzyme cleaves the insoluble collagen which unwinds and is thus solubilized. Collagenase activity is directly proportional to the amount of collagen solubilized, determined by the proportion of radioactivity released into the supernatant as measured in a standard scintillation counter. Collagenase inhibitors are, therefore, compounds which reduce the radioactive counts released with respect to the controls with no inhibitor present. One specific embodiment of this assay is described in detail below.

For determining the selectivity of compounds for MMP-13 versus MMP-1 using collagen as a substrate, the following procedure may be used. Recombinant human proMMP-13 or proMMP-1 is activated according to the procedures outlined above. The activated MMP-13 or MMP-1 is diluted to 0.6 μg/mL with buffer (50 mM Tris pH 7.5, 150 mM NaCl, 10 mM CaCl₂, 1 uM ZnCl₂, 0.05% Brij-35, 0.02% sodium azide).

Stock solutions of test compound (10 mM) in dimethylsulfoxide are prepared. Dilutions of the test compounds in the Tris buffer, above, are made to 0.2, 2.0, 20, 200, 2000 and 20000 nM.

One hundred microliters (100 μL) of appropriate drug dilution and 100 μL of diluted enzyme are pipetted into wells of a 96 well plate containing collagen films labeled with ¹⁴C-collagen. The final enzyme concentration is 0.3 μg/mL while the final drug concentration is 0.1, 1.0, 10, 100, 1000 nM. Each drug concentration and control is analyzed in triplicate. Triplicate controls are also run for the conditions in which no enzyme is present and for enzyme in the absence of any compound.

The plates are incubated at 37° C. for a time period such that around 30-50% of the available collagen is solubilized, as determined by counting additional control wells at various time points. In most cases around 9 hours of incubation are required. When the assay has progressed sufficiently, the supernatant from each well is removed and counted in a scintillation counter. The background counts (determined by the counts in the wells with no enzyme) are subtracted from each sample and the % release calculated in relation to the wells with enzyme only and no inhibitor. The triplicate values for each point are averaged and the data graphed as percent release versus drug concentration. IC₅₀'s are determined from the point at which 50% inhibition of release of radiolabeled collagen is obtained.

To determine the identity of the active collagenases in cartilage conditioned medium, assays were carried out using collagen as a substrate, cartilage conditioned medium containing collagenase activity and inhibitors of varying selectivity. The cartilage conditioned medium was collected during the time at which collagen degradation was occurring and thus is representative of the collagenases responsible for the collagen breakdown. Assays were carried out as outlined above except that instead of using recombinant MMP-13 or recombinant MMP-1, cartilage conditioned medium was the enzyme source.

Allosteric inhibitors of MMP-13 which are compounds of Formula I may be readily identified by assaying a test compound for inhibition of MMP-13 according to the methods described below in Biological Methods 5 and 6.

### Biological Method 5

Fluorigenic peptide-1 substrate based assay for identifying compounds of Formula I as allosteric inhibitors of MMP-13:

Final Assay Conditions:

50 mM HEPES buffer (pH 7.0)

10 mM CaCl₂

10 μM fluorigenic peptide-1 (“FP1”) substrate

0 or 15 mM acetohydroxamic acid (AcNHOH)=1 K_{d}

2% DMSO (with or without inhibitor test compound)

0.5 nM MMP-13CD enzyme

Stock Solutions:

1) 10× assay buffer: 500 mM HEPES buffer (pH 7.0) plus 100 mM CaCl₂

2) 10 mM FP1 substrate: (Mca)-Pro-Leu-Gly-Leu-(Dnp)-Dpa-Ala-Arg-NH₂ (Bachem, M-1895; “A novel coumarin-labeled peptide for sensitive continuous assays of the matrix metalloproteinases,” Knight C. G., Willenbrock F., and Murphy, G., FEBS Lett., 1992;296:263-266). Is prepared 10 mM stock by dissolving 5 mg FP1 in 0.457 mL DMSO.

3) 3 M AcNHOH: Is prepared by adding 4 mL H₂O and 1 mL 10× assay buffer to 2.25 g AcNHOH (Aldrich 15,903-4). Adjusting pH to 7.0 with NaOH. Diluting volume to 10 mL with H₂O. Final solution will contain 3 M AcNHOH, 50 mM HEPES buffer (pH 7.0), and 10 mM CaCl₂.

4) AcNHOH dilution buffer: 50 mM HEPES buffer (pH 7.0) plus 10 mM CaCl₂

5) MMP-13CD enzyme: Stock concentration=250 nM.

6) Enzyme dilution buffer: 50 mM HEPES buffer (pH 7.0), 10 mM CaCl₂, and 0.005% BRIJ 35 detergent (Calbiochem 203728; Protein Grade, 10%)

Procedure (For One 96-Well Microplate):

A. Prepared Assay Mixture:

1100 μL 10× assay buffer

11 μL 10 mM FP1

55 μL 3 M AcNHOH or 55 μL AcNHOH dilution buffer

8500 μL H₂O

B. Diluted MMP-13CD to 5 nM Working Stock:

22 μL MMP-13CD (250 nM)

1078 μL enzyme dilution buffer

C. Ran Kinetic Assay:

1. Dispense 2 μL inhibitor test sample (in 100% DMSO) into well.

2. Add 88 μL assay mixture and mix well, avoiding bubbles.

3. Initiate reactions with 10 μL of 5 nM MMP-13CD; mix well, avoid bubbles.

4. Immediately measure the kinetics of the reactions at room temperature.

Fluorimeter: Fₘₐₓ Fluorescence Microplate Reader & SOFTMAX PRO Version 1.1 software (Molecular Devices Corporation; Sunnyvale, Calif. 94089).

Protocol menu:

| excitation: | 320 nm | emission: | 405 nm |
|---|---|---|---|
| run time: | 15 min | interval: | 29 sec |
| RFU min: | −10 | RFU max: | 200 |
| Vₘₐₓ points: | 32/32 | | |

D. Compared % of control activity and/or IC₅₀ with inhibitor test compound ±AcNHOH.

Hydrolysis of the fluorigenic peptide-1 substrate, [(Mca)Pro-Leu-Gly-Leu-Dpa-Ala-Arg-NH₂; Bachem, catalog number M-1895], wherein “Mca” is (7-methoxy-coumarin-4-yl)acetyl and “Dpa” is (3-[2,4-dinitrophenyl]-L-2,3-diaminopropionyl), is used to screen for MMP-13 catalytic domain (CD) inhibitors. (Dpa may also be abbreviated as “Dnp”.) Reactions (100 μL) contain 0.05 M Hepes buffer (pH 7), 0.01 M calcium chloride, 0.005% polyoxyethylene (23) lauryl ether (“Brij 35”), 0 or 15 mM acetohydroxamic acid, 10 μM FP1, and 0.1 mM to 0.5 nM inhibitor in DMSO (2% final).

After recombinant human MMP-13CD (0.5 nM final) is added to initiate the reaction, the initial velocity of FP1 hydrolysis is determined by monitoring the increase in fluorescence at 405 nm (upon excitation at 320 nm) continuously for up to 30 minutes on a microplate reader at room temperature. Alternatively, an endpoint read can also be used to determine reaction velocity provided the initial fluorescence of the solution, as recorded before addition of enzyme, is subtracted from the final fluorescence of the reaction mixture. The inhibitor is assayed at different concentration values, such as, for example, 100 μM, 10 μM, 1 μM, 100 nM, 10 nM, and 1 nM. Then the inhibitor concentration is plotted on the X-axis against the percentage of control activity observed for inhibited experiments versus uninhibited experiments (i.e., (velocity with inhibitor) divided by (velocity without inhibitor)×100) on the Y-axis to determine IC₅₀ values. This determination is done for experiments done in the presence, and experiments done in the absence, of acetohydroxamic acid. Data are fit to the equation: percent control activity=100/[1+(([I]/IC₅₀)^{slope})], where [I] is the inhibitor concentration, IC₅₀ is the concentration of inhibitor where the reaction rate is 50% inhibited relative to the control, and slope is the slope of the IC₅₀ curve at the curve's inflection point, using nonlinear least-squares curve-fitting equation regression.

Results may be expressed as an IC₅₀ Ratio (+/−) ratio, which means a ratio of the IC₅₀ of the inhibitor with MMP-13 and an inhibitor to the catalytic zinc of MMP-13, divided by the IC₅₀ of the inhibitor with MMP-13 without the inhibitor to the catalytic zinc of MMP-13. Compounds of Formula I which are allosteric inhibitors of MMP-13 are expected to have an IC₅₀ Ratio (+/−) ratio of less than 1, and are expected to be synergistic with the inhibitor to the catalytic zinc of MMP-13 such as, for example, AcNHOH. Compounds of Formula I which are not allosteric inhibitors of MMP-13 will be inactive in the assay or will have an IC₅₀ Ratio (+/−) of greater than 1, unless otherwise indicated. Results can be confirmed by kinetics experiments which are well known in the biochemical art.

### Biological Method 6

Fluorigenic peptide-1 based assay for identifying allosteric compound inhibitors of matrix metalloproteinase-13 catalytic domain (“MMP-13CD”):

In a manner similar to Biological Method 5, an assay is run wherein 1,10-phenanthroline is substituted for acetohydroxamic acid to identify compounds of Formula I.

Other methods of assaying for inhibitor activity of a test compound with an MMP enzyme are described below in Biological Methods 7 to 10.

### Biological Method 7

Inhibition of Human Collagenase (MMP-1):

Human recombinant collagenase is activated with trypsin. The amount of trypsin is optimized for each lot of collagenase-1 but a typical reaction uses the following ratio: 5 μg trypsin per 100 μg of collagenase. The trypsin and collagenase are incubated at room temperature for 10 minutes then a five-fold excess (50 mg/10 mg trypsin) of soybean trypsin inhibitor is added.

Stock solutions (10 mM) of inhibitors are made up in dimethylsulfoxide and then diluted using the following scheme:

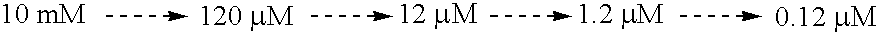

Twenty-five microliters of each concentration is then added in triplicate to appropriate wells of a 96 well microfluor plate. The final concentration of inhibitor will be a 1:4 dilution after addition of enzyme and substrate. Positive controls (enzyme, no inhibitor) are set up in wells D7-D12 and negative controls (no enzyme, no inhibitors) are set in wells D1-D6.

Collagenase-1 is diluted to 240 ng/mL and 25 μL is then added to appropriate wells of the microfluor plate. Final concentration of collagenase in the assay is 60 ng/mL.

Substrate (DNP-Pro-Cha-Gly-Cys(Me)-His-Ala-Lys(NMA)-NH₂) is made as a 5 mM stock in dimethylsulfoxide and then diluted to 20 μM in assay buffer. The assay is initiated by the addition of 50 μL substrate per well of the microfluor plate to give a final concentration of 10 μM.

Fluorescence readings (360 nM excitation, 460 nm emission) are taken at time 0 and then at 20 minute intervals. The assay is conducted at room temperature with a typical assay time of 3 hours

Fluorescence versus time is then plotted for both the blank and collagenase containing samples (data from triplicate determinations is averaged). A time point that provides a good signal (at least five fold over the blank) and that is on a linear part of the curve (usually around 120 minutes) is chosen to determine IC₅₀ values. The zero time is used as a blank for each compound at each concentration, and these values are subtracted from the 120 minute data. Data is plotted as inhibitor concentration versus % control (inhibitor fluorescence divided by fluorescence of collagenase alone×100). IC₅₀'s are determined from the concentration of inhibitor that gives a signal that is 50% of the control.

If IC₅₀'s are reported to be less than 0.03 μM then the inhibitors are assayed at concentrations of 0.3 μM, 0.03 μM, and 0.003 μM.

### Biological Method 8

Inhibition of Gelatinase (MMP-2):

Human recombinant 72 kD gelatinase (MMP-2, gelatinase A) is activated for 16-18 hours with 1 mM p-aminophenyl-mercuric acetate (from a freshly prepared 100 mM stock in 0.2 N NaOH) at 4° C., rocking gently. 10 mM dimethylsulfoxide stock solutions of inhibitors are diluted serially in assay buffer (50 mM TRIS, pH 7.5, 200 mM NaCl, 5 mM CaCl₂, 20 μM ZnCl₂ and 0.02% BRIJ-35 (vol./vol.)) using the following scheme:

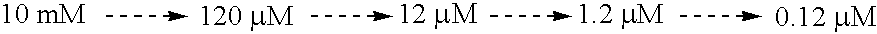

Further dilutions are made as necessary following this same scheme. A minimum of four inhibitor concentrations for each compound are performed in each assay. 25 μL of each concentration is then added to triplicate wells of a black 96 well U-bottomed microfluor plate. As the final assay volume is 100 μL, final concentrations of inhibitor are the result of a further 1:4 dilution (i.e. 30 μM→3 μM→0.3 μM→0.03 μM, etc.). A blank (no enzyme, no inhibitor) and a positive enzyme control (with enzyme, no inhibitor) are also prepared in triplicate.

Activated enzyme is diluted to 100 ng/mL in assay buffer, 25 μL per well is added to appropriate wells of the microplate. Final enzyme concentration in the assay is 25 ng/mL (0.34 nM).

A five mM dimethylsulfoxide stock solution of substrate (Mca-Pro-Leu-Gly-Leu-Dpa-Ala-Arg-NH₂) is diluted in assay buffer to 20 μM. The assay is initiated by addition of 50 μL of diluted substrate yielding a final assay concentration of 10 μM substrate. At time zero, fluorescence reading (320 excitation; 390 emission) is immediately taken and subsequent readings are taken every fifteen minutes at room temperature with a PerSeptive Biosystems CytoFluor Multi-Well Plate Reader with the gain at 90 units.

The average value of fluorescence of the enzyme and blank are plotted versus time. An early time point on the linear part of this curve is chosen for IC₅₀ determinations. The zero time point for each compound at each dilution is subtracted from the latter time point and the data then expressed as percent of enzyme control (inhibitor fluorescence divided by fluorescence of positive enzyme control ×100). Data is plotted as inhibitor concentration versus percent of enzyme control. IC₅₀'s are defined as the concentration of inhibitor that gives a signal that is 50% of the positive enzyme control.

### Biological Method 9

Inhibition of Stromelysin Activity (MMP-3):

Human recombinant stromelysin (MMP-3, stromelysin-1) is activated for 20-22 hours with 2 mM p-aminophenyl-mercuric acetate (from a freshly prepared 100 mM stock in 0.2 N NaOH) at 37° C. 10 mM dimethylsulfoxide stock solutions of inhibitors are diluted serially in assay buffer (50 mM TRIS, pH 7.5, 150 mM NaCl, 10 mM CaCl₂ and 0.05% BRIJ-35 (vol./vol.)) using the following scheme:

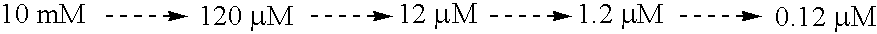

Further dilutions are made as necessary following this same scheme. A minimum of four inhibitor concentrations for each compound are performed in each assay. 25 μL of each concentration is then added to triplicate wells of a black 96 well U-bottomed microfluor plate. As the final assay volume is 100 μL, final concentrations of inhibitor are the result of a further 1:4 dilution (i.e. 30 μM→3 μM→0.3 μM→0.03 μM, etc.). A blank (no enzyme, no inhibitor) and a positive enzyme control (with enzyme, no inhibitor) are also prepared in triplicate.

Activated enzyme is diluted to 200 ng/mL in assay buffer, 25 μL per well is added to appropriate wells of the microplate. Final enzyme concentration in the assay is 50 ng/mL (0.875 nM).

A 10 mM dimethylsulfoxide stock solution of substrate (Mca-Arg-Pro-Lys-Pro-Val-Glu-Nva-Trp-Arg-Lys(Dnp)-NH₂) is diluted in assay buffer to 6 μM. The assay is initiated by addition of 50 μL of diluted substrate yielding a final assay concentration of 3 μM substrate. At time zero, fluorescence reading (320 excitation; 390 emission) is immediately taken and subsequent readings are taken every fifteen minutes at room temperature with a PerSeptive Biosystems CytoFluor Multi-Well Plate Reader with the gain at 90 units.

The average value of fluorescence of the enzyme and blank are plotted versus time. An early time point on the linear part of this curve is chosen for IC₅₀ determinations. The zero time point for each compound at each dilution is subtracted from the latter time point and the data then expressed as percent of enzyme control (inhibitor fluorescence divided by fluorescence of positive enzyme control ×100). Data is plotted as inhibitor concentration versus percent of enzyme control. IC₅₀'s are defined as the concentration of inhibitor that gives a signal that is 50% of the positive enzyme control.

### Biological Method 10

Inhibition of Human 92 kD Gelatinase (MMP-9):

Inhibition of 92 kD gelatinase (MMP-9) activity is assayed using the Mca-Pro-Leu-Gly-Leu-Dpa-Ala-Arg-NH₂ substrate (10 μM) under similar conditions as described above for the inhibition of human collagenase (MMP-1).

Human recombinant 92 kD gelatinase (MMP-9, gelatinase B) is activated for 2 hours with 1 mM p-aminophenyl-mercuric acetate (from a freshly prepared 100 mM stock in 0.2 N NaOH) at 37 C.

10 mM dimethylsulfoxide stock solutions of inhibitors are diluted serially in assay buffer (50 mM TRIS, pH 7.5, 200 mM NaCl, 5 mM CaCl₂, 20 μM ZnCl₂, 0.02% BRIJ-35 (vol./vol.)) using the following scheme:

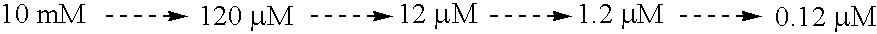

Further dilutions are made as necessary following this same scheme. A minimum of four inhibitor concentrations for each compound are performed in each assay. 25 μL of each concentration is then added to triplicate wells of a black 96 well U-bottomed microfluor plate. As the final assay volume is 100 μL, final concentrations of inhibitor are the result of a further 1:4 dilution (i.e. 30 μM→3 μM→0.3 μM→0.03 μM, etc.). A blank (no enzyme, no inhibitor) and a positive enzyme control (with enzyme, no inhibitor) are also prepared in triplicate.

Activated enzyme is diluted to 100 ng/mL in assay buffer, 25 μL per well is added to appropriate wells of the microplate. Final enzyme concentration in the assay is 25 ng/m]L (0.27 nM).

A 5 mM dimethylsulfoxide stock solution of substrate (Mca-Pro-Leu-Gly-Leu-Dpa-Ala-Arg-NH₂) is diluted in assay buffer to 20 μM. The assay is initiated by addition of 50 μL of diluted substrate yielding a final assay concentration of 10 μM substrate. A 0-time fluorescence reading (320 excitation; 390 emission) is immediately taken and subsequent readings are taken every fifteen minutes at room temperature with a PerSeptive Biosystems CytoFluor Multi-Well Plate Reader with the gain at 90 units.

The average value of fluorescence of the enzyme and blank are plotted versus time. An early time point on the linear part of this curve is chosen for IC₅₀ determinations. The 0 time point for each compound at each dilution is subtracted from the latter time point and the data then expressed as percent of enzyme control (inhibitor fluorescence divided by fluorescence of positive enzyme control ×100). Data is plotted as inhibitor concentration versus percent of enzyme control. IC₅₀'s are defined as the concentration of inhibitor that gives a signal that is 50% of the positive enzyme control.

2. In Vivo Biological Methods

Animal models may be used to establish that the instant compounds of Formula I, or a pharmaceutically acceptable salt thereof, are useful for treating osteoarthritis, cartilage damage, rheumatoid arthritis, and breast cancer. For example, animal models for preventing, treating, and inhibiting cartilage damage, and thus for preventing or treating osteoarthritis are described below in Biological Methods 11 to 13. The ability or inability of the compounds or the pharmaceutically acceptable salts thereof to inhibit the production of TNF may be determined according to the method of Biological Method 14. The invention compounds may also be assayed for their ability to inhibit aggrecanase-mediated release of proteoglycan in IL-1 stimulated cells, as described below in Biological Method 15.

### Biological Method 11

Monosodium Iodoacetate-Induced Osteoarthritis in Rat Model of Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions.

Generally, In the MIA Rat model on Day 0, the hind-paw weight differential between the right arthritic joint and the left healthy joint of male Wistar rats (150 g) are determined with an incapacitance tester, model 2KG (Linton Instrumentation, Norfolk, United Kingdom). The incapacitance tester has a chamber on top with an outwardly sloping front wall that supports a rat's front limbs, and two weight sensing pads, one for each hind paw, that facilitates this determination. Then the rats are anesthetized with isofluorine, and the right, hind leg knee joint is injected with 1.0 mg of mono-iodoacetate (“MIA”) through the infrapatellar ligament. Injection of MIA into the joint results in the inhibition of glycolysis and eventual death of surrounding chondrocytes. The rats are further administered either an invention compound or vehicle (in the instant case, water) daily for 14 days or 28 days.

The invention compound is typically administered at a dose of 30 mg per kilogram of rat per day (30 mg/kg/day), but the invention compound may be administered at other doses such as, for example, 10 mg/kg/day, 60 mg/kg/day, 90-mg/kg/day, or 100 mg/kg/day according to the requirements of the compound being studied. It is well within the level of ordinary skill in the pharmaceutical arts to determine a proper dosage of an invention compound in this model. Administration of the invention compound in this model is optionally by oral administration or intravenous administration via an osmotic pump.

After 7 and 14 days for a two-week study, or 7, 14, and 28 days for a four-week study, the hind-paw weight distribution is again determined. Typically, the animals administered vehicle alone place greater weight on their unaffected left hind paw than on their right hind paw, while animals administered an invention compound show a more normal (i.e., more like a healthy animal) weight distribution between their hind paws. This change in weight distribution was proportional to the degree of joint cartilage damage. Percent inhibition of a change in hind paw joint function is calculated as the percent change in hind-paw weight distribution for treated animals versus control animals. For example, for a two week study,

Percent inhibition of a change in hind paw joint function

$$Percent inhibition of a change in hind paw joint$$

$$function = \{ 1 - [ \frac{( Δ {W}_{G} )}{( Δ {W}_{C} )} ] \} \times 100$$

wherein:

ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an invention compound, as measured on Day 14.

In order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an invention compound, and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {[(proteoglycan loss from joint (%) with vehicle)−(proteoglycan loss from joint with an invention compound)]÷(proteoglycan loss from joint (%) with vehicle)}×100.

The MIA Rat data that are expected from the analysis of proteoglycan loss would establish that an invention compound is effective for inhibiting cartilage damage and inflammation and/or alleviating pain in mammalian patients, including human.

The results of these studies with oral dosing may be presented in tabular format in the columns labelled “IJFL (%+/−SEM)”, wherein IJFL means Inhibition of Joint Function Limitation, “SDCES”, wherein SDCES means Significant Decrease In Cartilage Erosion Severity, and “SIJWHLE”, wherein SIJWHLE means Significant Increase in Joints Without Hind Limb Erosion.

The proportion of subjects without hind limb erosions may be analyzed via an Exact Sequential Cochran-Armitage Trend test (SAS® Institute, 1999). The Cochran-Armitage Trend test is employed when one wishes to determine whether the proportion of positive or “Yes” responders increases or decreases with increasing levels of treatment. For the particular study, it is expected that the number of animals without joint erosions increased with increasing dose.

The ridit analysis may be used to determine differences in overall erosion severity. This parameter takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

Another animal model for measuring effects of an invention compound on cartilage damage and inflammation and/or pain is described below in Biological Method 12.

### Biological Method 12

Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or an invention compound dosed three times per day with 30-mg/kg/dose or 10-mg/kg/dose. The invention compound may be administered at other doses such as, for example, 3 times 20 mg/kg/day or 3 times 60 mg/kg/day according to the requirements of the invention compound being studied. The rabbits are euthanized 8 weeks after surgery and the proximal end of the tibia and the distal end of the femur are removed from each animal.

Macroscopic Grading:

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading:

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe histologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis:

Mean values and SEM is calculated and statistical analysis was done using the Mann-Whitney U-test.

The results of these studies would be expected to show that an invention compound would reduce the size of the lesion on the tibial plateaus, and perhaps the damage in the tibia or on the femoral condyles. In conclusion, these results would show that an invention compound would have significant inhibition effects on the damage to cartilage.

### Biological Method 13

IL-1 Induced Cartilage Collagen Degradation from Bovine Nasal Cartilage:

This assay uses bovine nasal cartilage explants which are commonly used to test the efficacy of various compounds to inhibit either IL-1 induced proteoglycan degradation or IL-1 induced collagen degradation. Bovine nasal cartilage is a tissue that is very similar to articular cartilage, i.e. chondrocytes surrounded by a matrix that is primarily type II collagen and aggrecan. The tissue is used because it: (1) is very similar to articular cartilage, (2) is readily available, (3) is relatively homogeneous, and (4) degrades with predictable kinetics after IL-1 stimulation.

Two variations of this assay have been used to assay compounds. Both variations give similar data. The two variations are described below:

### Variation 1

Three plugs of bovine nasal cartilage (approximately 2 mm diameter×1.5 mm long) are placed into each well of a 24 well tissue culture plate. One mL of serumless medium is then added to each well. Compounds are prepared as 10 mM stock solutions in DMSO and then diluted appropriately in serumless medium to final concentrations, e.g., 50, 500 and 5000 nM. Each concentration is assayed in triplicate.

Human recombinant IL-1α (5 ng/mL) (IL-1) is added to triplicate control wells and to each well containing drug. Triplicate control wells are also set up in which neither drug nor IL-1 are added. The medium is removed, and fresh medium containing IL-1 and the appropriate drug concentrations is added on days 6, 12, 18 and 24 or every 3-4 days if necessary. The media removed at each time point is stored at −20° C. for later analysis. When the cartilage in the IL-1 alone wells has almost completely resorbed (about Day 21), the experiment is terminated. The medium is removed and stored. Aliquots (100 μL) from each well at each time point are pooled, digested with papain and then analyzed for hydroxyproline content. Background hydroxyproline (average of wells with no IL-1 and no drug) is subtracted from each data point and the average calculated for each triplicate. The data is then expressed as a percent of the IL-1 alone average value and plotted. The IC₅₀ is determined from this plot.

### Variation 2

The experimental set-up is the same as outlined above in Variation 1, until day 12. On day 12, the conditioned medium from each well is removed and frozen. Then 1 mL of phosphate buffered saline (PBS) containing 0.5 μg/mL trypsin is added to each well and incubation continued for a further 48 hours at 37° C. After 48 hours incubation in trypsin, the PBS solution is removed. Aliquots (50 μl) of the PBS/trypsin solution and the previous two time points (Days 6 and 12) are pooled, hydrolyzed and hydroxyproline content determined. Background hydroxyproline (average of wells with no IL-1 and no drug) is subtracted from each data point and the average calculated for each triplicate. The data is then expressed as a percent of the IL-1 alone average value and plotted. The IC₅₀ is determined from this plot.

In this variation, the time course of the experiment is shortened considerably. The addition of trypsin for 48 hours after 12 days of IL-1 stimulation likely releases any type II collagen that has been damaged by collagenase activity but not yet released from the cartilage matrix. In the absence of IL-1 stimulation, trypsin treatment produces only low background levels of collagen degradation in the cartilage explants.

The invention compounds may also be assayed for their ability to inhibit production of tumor necrosis factor alpha (“TNF”) as described below in Biological Method 14.

### Biological Method 14

Inhibition of TNF Production:

The ability or inability of the compounds or the pharmaceutically acceptable salts thereof to inhibit the production of TNF is shown by the following in vitro assay:

### Human Monocyte Assay

Human mononuclear cells were isolated from anti-coagulated human blood using a one-step Ficoll-hypaque separation technique. (2) The mononuclear cells were washed three times in Hanks balanced salt solution (HBSS) with divalent cations and resuspended to a density of 2×10⁶/mL in HBSS containing 1% BSA. Differential counts determined using the Abbott Cell Dyn 3500 analyzer indicated that monocytes ranged from 17 to 24% of the total cells in these preparations. 180 μL of the cell suspension was aliquoted into flat bottom 96 well plates (Costar). Additions of compounds and LPS (100 ng/mL final concentration) gave a final volume of 200 μL. All conditions were performed in triplicate. After a four hour incubation at 37° C. in an humidified CO₂ incubator, plates were removed and centrifuged (10 minutes at approximately 250×g) and the supernatants removed and assayed for TNF α using the R&D ELISA Kit.

The invention compounds may also be assayed for their ability to inhibit aggrecanase-mediated release of proteoglycan in IL-1 stimulated cells, as described below in Biological Method 15.

### Biological Method 15

Aggrecanase Assay:

Primary porcine chondrocytes from articular joint cartilage are isolated by sequential trypsin and collagenase digestion followed by collagenase digestion overnight and are plated at 2×10⁵ cells per well into 48 well plates with 5 μCi/mL ³⁵S (1000 Ci/mmol) sulphur in type I collagen coated plates. Cells are allowed to incorporate label into their proteoglycan matrix (approximately 1 week) at 37° C., under an atmosphere of 5% CO₂.

The night before initiating the assay, chondrocyte monolayers are washed two times in DMEM/1% PSF/G and then allowed to incubate in fresh DMEM/1% FBS overnight.

The following morning chondrocytes are washed once in DMEM/1% PSF/G. The final wash is allowed to sit on the plates in the incubator while making dilutions.

Media and dilutions can be made as described in the table below.

| | |
|---|---|
| Control | DMEM alone (control media) |
| Media | |
| IL-1 Media | DMEM + IL-1 (5 ng/mL) |
| Drug | Make all compounds stocks at 10 mM in DMSO. |
| Dilutions | Make a 100 uM stock of each compound in DMEM in 96 |
| | well plate. Store in freezer overnight. |
| | The next day perform serial dilutions in DMEM with IL-1 |
| | to 5 uM, 500 nM, and 50 nM. |
| | Aspirate final wash from wells and add 50 μL of |
| | compound from above dilutions to 450 μL of IL-1 media in |
| | appropriate wells of the 48 well plates. |
| | Final compound concentrations equal 500 nM, 50 nM, and |
| | 5 nM. All samples completed in triplicate with Control |
| | and IL-1 alone samples on each plate. |

Plates are labeled and only the interior 24 wells of the plate are used. On one of the plates, several columns are designated as IL-1 (no drug) and Control (no IL-1, no drug). These control columns are periodically counted to monitor ³⁵S-proteoglycan release. Control and IL-1 media are added to wells (450 μL) followed by compound (50 mL) so as to initiate the assay. Plates are incubated at 37° C., with a 5% CO₂ atmosphere.

At 40-50% release (when CPM from IL-1 media is 4-5 times control media) as assessed by liquid scintillation counting (LSC) of media samples, the assay is terminated (9-12 hours). Media is removed from all wells and placed in scintillation tubes. Scintillate is added and radioactive counts are acquired (LSC). To solubilize cell layers, 500 μL of papain digestion buffer (0.2 M Tris, pH 7.0, 5 mM EDTA, 5 mM DTT, and 1 mg/mL papain) is added to each well. Plates with digestion solution are incubated at 60° C. overnight. The cell layer is removed from the plates the next day and placed in scintillation tubes. Scintillate is then added, and samples counted (LSC).

The percent of released counts from the total present in each well is determined. Averages of the triplicates are made with control background subtracted from each well. The percent of compound inhibition is based on IL-1 samples as 0% inhibition (100% of total counts).

Biological Data for Invention Compounds

The invention compounds were evaluated in standard assays of Biological Methods 1, 2, or 5 for their ability to specifically inhibit the catalytic activity of MMP-13, particularly MMP-13 catalytic domain (“MMP-13CD”) over MMP-1 full-length (“MMP-1FL”), MMP-3 catalytic domain (“MMP-3CD”), MMP-7 full-length (“MMP-7FL”), MMP-8 full-length (“MMP-8FL”), MMP-9 full-length (“MMP-9FL”), MMP-12 catalytic domain (“MMP-12CD”), MMP-14 catalytic domain (“MMP-14CD”), and MMP-17 catalytic domain (“MMP-17CD”). Shown below in Table 1 are the inhibition of MMP-13 catalytic domain, expressed as IC₅₀'s, for the compounds of Compound Examples A1 to A8, B1 to B4, and C1 in the column labelled “MMP-13CD IC₅₀ (μM)”.

TABLE 1

| Compound | |
|---|---|
| Example | MMP-13CD |
| No. | IC₅₀ (μM) |
| A1 | 0.038 |
| A2 | 0.053 |
| A3 | 0.035 |
| A4 | 0.036 |
| A5 | 0.25 |
| A6 | 0.0053 |
| A7 | 0.10 |
| A8 | 0.021 |
| B1 | 0.053 |
| B2 | 0.013 |
| B3 | 0.292 |
| B4 | 0.048 |

Not shown in Table 1, the IC₅₀'s of the compounds of Compound Examples A1, B1, B2, B3, and B4, each with MMP-3CD were each greater than 65 μM, and with MMP-8FL, MMP-9FL, MMP-12CD, MMP-14CD, or MMP-17CD are each greater than 30 μM. The IC₅₀'s of the compound of Compound Example A2 with MMP-1FL, MMP-3CD, MMP-7FL, MMP-8FL, MMP-14CD, or MMP-17CD was each greater than 100 μM. The IC₅₀'s of the compounds of Compound Example A3, A4, and A5, each with MMP-1FL, MMP-3CD, MMP-7FL, MMP-8FL, MMP-9FL, or MMP-17CD were each greater than 100 μM, and with MMP-14CD were each greater than 30 μM. The compounds of Compound Examples A1 to A5 and B1 to B4 have been thus been shown to be specific inhibitors of MMP enzymes.

Selectivities of the invention compounds for MMP-13 versus another MMP enzymes were determined with the above data by dividing the IC₅₀ for an invention compound with a comparator MMP enzyme by the IC₅₀ of the invention compound with MMP-13. Selectivities of the invention compounds determined using the data provided above ranged from 50 fold to greater than 5,000 fold potency with MMP-13 versus with at least six other MMP enzymes.

The foregoing data establish that the invention compounds are potent and specific inhibitors of MMP-13. Because of this potency and specificity for MMP-13, the invention compounds are especially useful to treat diseases mediated by MMP-13 enzymes, and including those mediated by human MMP-13 such as osteoarthritis, cartilage damage, rheumatoid arthritis, and breast cancer.

If the invention compounds were to be tested in the in vivo assays described above, the in vivo studies would establish that an invention compound is effective for the inhibition of cartilage damage and inflammation and/or alleviating pain, and thus useful for the treatment of osteoarthritis or rheumatoid arthritis, improving joint function, reducing joint stiffness, in human and other mammals. As described above, such a treatment offers a distinct advantage over existing treatments that only modify pain or inflammation or and other secondary symptoms. The effectiveness of an invention compound in this model would indicate that the invention compound will have clinically useful effects in preventing and/or treating cartilage damage, pain and/or inflammation.

Administration of Invention Compounds:

For these purposes, the compounds of the present invention can be prepared and administered to patients in a wide variety of dosage forms, including oral, parenteral, and the like. Thus, the compounds of the present invention can be administered orally, buccally, sublingually, transdermally, topically to the skin, mucosa, dermally or transdermally, intranasally, rectally, by vaginal route, occularly, by inhalation, by injection, that is, intravenously, intramuscularly, intracutaneously, intraduodenally, intraperitoneally, intraarterially, intrathecally, intraventicularly, intraurethrally intrastemally, intracranally, intraspinally, or subcutaneously, or they may be administered by infusion, needle-free injectors, or implant injection techniques. Also, the compounds of the present invention can be administered by inhalation, for example, intranasally. It will be obvious to those skilled in the art that the following dosage forms may comprise as the active component, either a compound of Formula I, or a corresponding pharmaceutically acceptable salt thereof. The active compound generally is present in a concentration of about 5% to about 95% by weight of the formulation.

Administration includes delivery to the patient by viral or non-viral techniques. Viral delivery mechanisms include, but are not limited to, adenoviral vectors, adeno-associated viral (“AAV”) vectors, herpes viral vectors, retroviral vectors, lentiviral vectors, and baculoviral vectors. Non-viral delivery mechanisms include lipid-mediated transfection, liposomes, immunoliposomes, lipofectin, cationic facial amphiphiles (“CFAs”), and combinations thereof.

The invention compounds may be administered to a patient alone or in a pharmaceutical composition comprising a sufficiently nontoxic, therapeutically effective amount of the compound. The invention compounds may also be used in combination with a cyclodextrin, which are known to form inclusion and non-inclusion complexes with drugs that may modify solubility, dissolution rate, taste-masking, bioavailability, and/or a stability property of a drug.

A sufficiently nontoxic, therapeutically effective amount, or, simply, effective amount, of an invention compound will generally be from about 1 to about 300 mg/kg/day of subject body weight of the compound of Formula I, or a pharmaceutically acceptable salt thereof. Typical doses will be from about 10 to about 5000 mg/day for an adult subject of normal weight for the invention compound or each component of an invention combination. In a clinical setting, regulatory agencies such as, for example, the Food and Drug Administration (“FDA”) in the U.S. may require a particular therapeutically effective amount.

In determining what constitutes a sufficiently nontoxic therapeutically effective amount of an invention compound for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, ie, either below or above those ranges, depending upon the requirements of the individual subject, the severity of the condition being treated, and the particular therapeutic formulation being employed.

Determination of a proper dose for a particular situation is within the skill of the medical or veterinary arts. Generally, treatment may be initiated using smaller dosages of the invention compound that are less than optimum for a particular subject. Thereafter, the dosage can be increased by small increments until the optimum effect under the circumstance is reached. For convenience, the total daily dosage may be divided and administered in portions during the day, if desired.

Preferred routes of administration of an invention compound are oral or parenteral. However, another route of administration may be preferred depending upon the condition being treated. For exampled, topical administration or administration by injection may be preferred for treating conditions localized to the skin or a joint. Administration by transdermal patch may be preferred where, for example, it is desirable to effect sustained dosing.

It should be appreciated that the different routes of administration may require different dosages. For example, a useful intravenous (“IV”) dose is between 5 and 50 mg, and a useful oral dosage is between 20 and 800 mg, of a compound of Formula I, or a pharmaceutically acceptable salt thereof. The dosage is within the dosing range used in treatment of the above-listed diseases, or as would be determined by the needs of the patient as described by the physician.

The invention compounds may be administered in any pharmaceutically acceptable form. Preferably, administration is in unit dosage form. A unit dosage form of the invention compound to be used in this invention may also comprise other compounds useful in the therapy of diseases described above.

In therapeutic use as agents to inhibit a matrix metalloproteinase enzyme for the treatment of atherosclerotic plaque rupture, aortic aneurism, heart failure, restenosis, periodontal disease, corneal ulceration, cancer metastasis, tumor angiogenesis, arthritis, or other autoimmune or inflammatory disorders dependent upon breakdown of connective tissue, the compounds utilized in the pharmaceutical method of this invention are administered at a dose that is effective to inhibit the hydrolytic activity of one or more matrix metalloproteinase enzymes. The initial dosage of about 1 mg/kg to about 100 mg/kg daily will be effective. A daily dose range of about 25 mg/kg to about 75 mg/kg is preferred.

The dosages, however, may be varied depending upon the requirements of the patient, the severity of the condition being treated, and the compound being employed. Generally, treatment is initiated with smaller dosages that are less than the optimum dose of the compound. Thereafter, the dosage is increased by small increments until the optimum effect under the circumstance is reached.

For convenience, the total daily dosage may be divided and administered in portions during the day if desired. Typical dosages will be from about 0.1 mg/kg to about 500 mg/kg, and ideally about 25 mg/kg to about 250 mg/kg, such that it will be an amount that is effective to treat the particular disease being prevented or controlled.

The active components of invention combinations, may be formulated together or separately and may be administered together or separately. The particular formulation and administration regimens used may be tailored to the particular patient and condition being treated by a practitioner of ordinary skill in the medical or pharmaceutical arts.

Formulation of Invention Compounds and Combinations:

An invention pharmaceutical composition may be produced by formulating the invention compound or combination with a pharmaceutically acceptable carrier. Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the active ingredients of the invention combinations may be formulated separately.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component.

In tablets, the active component is mixed with the carrier having the necessary binding properties in suitable proportions and compacted in the shape and size desired.

The powders and tablets preferably contain from 5% or 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be prepared by dispersing the finely divided active component in water with viscous material, such as natural or synthetic gums, resins, methylcellulose, sodium carboxymethylcellulose, and other well-known suspending agents.

Also included in the invention are solid form preparations that are intended to be converted, shortly before use, to liquid form preparations for oral or intravenous administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In a unit dosage form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 1 to 1000 mg, preferably 10 to 100 mg according to the particular application and the potency of the active component. The composition can, if desired, also contain other compatible therapeutic agents as described above.

The percentage of the active ingredients of a compound of Formula I, or a pharmaceutically acceptable salt thereof, in the foregoing compositions can be varied within wide limits, but for practical purposes it is preferably present in a total concentration of at least 10% in a solid composition and at least 2% in a primary liquid composition. The most satisfactory compositions are those in which a much higher proportion of the active ingredients are present, for example, up to about 95%.

It should be appreciated that determination of proper dosage forms, dosage amounts, and routes of administration, is within the level of ordinary skill in the pharmaceutical and medical arts.

The following formulation examples illustrate typical formulations provided by the invention.

### Formulation Example 1

Tablet Formulation

| Ingredient | Amount (mg) |
|---|---|
| Compound of Example A1 | 25 |
| Lactose | 50 |
| Corn starch (for mix) | 10 |
| Corn starch (paste) | 10 |
| Magnesium stearate (1%) | 5 |
| Total | 100 |

The amide of Compound Example A1, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of breast cancer, osteoarthritis, cartilage damage, or rheumatoid arthritis.

### Formulation Example 2

Preparation for Oral Solution

| Ingredient | Amount | |
|---|---|---|
| Compound of Example B1 | 400 | mg |
| Sorbitol solution (70% N.F.) | 40 | mL |
| Sodium benzoate | 20 | mg |
| Saccharin | 5 | mg |
| Red dye | 10 | mg |
| Cherry flavor | 20 | mg |
| Distilled water q.s. | 100 | mL |

The sorbitol solution is added to 40 mL of distilled water, and the amide of Compound Example B 1 is dissolved therein. The saccharin, sodium benzoate, flavor, and dye are added and dissolved. The volume is adjusted to 100 mL with distilled water. Each milliliter of syrup contains 4 mg of the invention compound.

### Formulation Example 3

Parenteral Solution

In a solution of 700 mL of propylene glycol and 200 mL of water for injection is suspended 20 g of the compound of Compound Example C1. After suspension is complete, the pH is adjusted to 6.5 with 1N sodium hydroxide, and the volume is made up to 1000 mL with water for injection. The formulation is sterilized, filled into 5.0-mL ampoules each containing 2.0 mL, and sealed under nitrogen.

As matrix metalloproteinase inhibitors, the invention compounds are useful as agents for the treatment of breast cancer, cartilage damage, osteoarthritis, and rheumatoid arthritis. They are also useful as agents for the treatment of multiple sclerosis, atherosclerotic plaque rupture, restenosis, periodontal disease, corneal ulceration, treatment of burns, decubital ulcers, wound repair, heart failure, cancer metastasis, tumor angiogenesis, arthritis, and other inflammatory disorders dependent upon tissue invasion by leukocytes. They are also useful for treating other diseases that are described above.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All of the references cited above are hereby incorporated by reference herein in their entireties and for all purposes.

Having described the invention, various embodiments of the invention are hereupon claimed.

What is claimed is:

1. A compound of Formula II 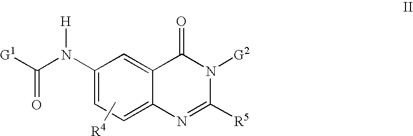
or a pharmaceutically acceptable salt thereof, wherein:
Each G¹ and G² independently is an unsubstituted or substituted group selected from:
C₃ to C₇ cycloalkyl-(C₁-C₈ alkylenyl)ₘ-;
C₅ or C₆ cycloalkyl-(C₁-C₈ alkylenyl)ₘ-;
C₈-C₁₀ bicycloalkyl-(C₁-C₈ alkylenyl)ₘ-;
3- to 7-membered heterocycloalkyl-(C₁-C₈ alkylenyl)ₘ-;
5- or 6-membered heterocycloalkyl-(C₁-C₈ alkylenyl)ₘ-;
8- to 10-membered heterobicycloalkyl-(C₁-C₈ alkylenyl)ₘ-;
Phenyl-(C₁-C₈ alkylenyl)ₘ-;
Naphthyl-(C₁-C₈ alkylenyl)ₘ-;
5- or 6-membered heteroaryl-(C₁-C₈ alkylenyl)ₘ-;
8- to 10-membered heterobiaryl-(C₁-C₈ alkylenyl)ₘ-;
5- or 6-membered heterocycloalkyl-phenylenyl-(C₁-C₈ alkylenyl)ₘ-;
Biphenyl-(C₁-C₈ alkylenyl)ₘ-;
5- or 6-membered heteroaryl-phenylenyl-(C₁-C₈ alkylenyl)ₘ-;
5- or 6-membered heteroaryl-(5- or 6-membered heteroarylenyl)-(C₁-C₈ alkylenyl)ₘ-;
Phenyl-L-(phenylenyl)-(C₁-C₈ alkylenyl)ₘ-;
Phenyl-L-(5- or 6-membered heteroarylenyl)-(C₁-C₈ alkylenyl)ₘ-;
8- to 10-membered heterobiaryl-phenylenyl-(C₁-C₈ alkylenyl)ₘ-;
Phenyl-(5- or 6-membered heteroarylenyl)-(C₁-C₈ alkylenyl)ₘ-;
Naphthyl-(5- or 6-membered heteroarylenyl)-(C₁-C₈ alkylenyl)ₘ-;
Phenyl-O—(C₁-C₈ alkylenyl)ₘ-;
Phenyl-S—(C₁-C₈ alkylenyl)ₘ-;
Phenyl-S(O)—(C₁-C₈ alkylenyl)ₘ-;
Phenyl-S(O)₂—(C₁-C₈ alkylenyl)ₘ-;
Phenyl-(8- to 10-membered heterobiarylenyl)-(C₁-C₈ alkylenyl)ₘ-;
and any of the above G¹ and G² groups independently substituted with from 1 to 6 substituents, each independently on a carbon or nitrogen atom, independently selected from:
C₁-C₆ alkyl-(G)ₘ-;
C₁-C₆ alkyl;
CN;
CF₃;
HO;
(C₁-C₆ alkyl)-O;
(C₁-C₆ alkyl)-S;
(C₁-C₆ alkyl)-S(O);
(C₁-C₆ alkyl)-S(O)₂;
O₂N;
H₂N;
(C₁-C₆ alkyl)-N(H);
(C₁-C₆ alkyl)₂—N;
(C₁-C₆ alkyl)-C(O)O-(C₁-C₈ alkylenyl)ₘ-;
(C₁-C₆ alkyl)-C(O)O-(1- to 8-membered heteroalkylenyl)ₘ-;
(C₁-C₆ alkyl)-C(O)N(H)—(C₁-C₈ alkylenyl)ₘ-;
(C₁-C₆ alkyl)-C(O)N(H)-(1- to 8-membered heteroalkylenyl)ₘ-;
H₂NS(O)₂—(C₁-C₈ alkylenyl)ₘ-;
(C₁-C₆ alkyl)-N(H)S(O)₂—(C₁-C₈ alkylenyl)ₘ-;
(C₁-C₆ alkyl)₂—NS(O)₂—(C₁-C₈ alkylenyl)ₘ-;
3- to 6-membered heterocycloalkyl-(G)ₘ-;
5- or 6-membered heteroaryl-(G)ₘ-;
(C₁-C₆ alkyl)-S(O)₂—N(H)—C(O)—(C₁-C₈ alkylenyl)ₘ-; and
(C₁-C₆ alkyl)-C(O)—N(H)—S(O)₂—(C₁-C₈ alkylenyl)ᵣ—;
wherein each substituent on a carbon atom of G¹ or G² may further be independently selected from Halo and HO₂C;
wherein 2 substituents on the same carbon atom of G¹ or G² may be taken together with the carbon atom to which they are both bonded to form the group C═O;
wherein G¹ and G² are not both independently selected from phenyl, benzyl, naphthyl, C₃ to C₇ cycloalkyl-(C₁-C₈ alkenyl)ₘ-, C₈-C₁₀ bicycloalkyl-(C₁-C₈ alkenyl)ₘ-, 3- to 7-membered heterocycloalkyl-(C₁-C₈ alkenyl)ₘ-, and 8- to 10-membered heterobicycloalkyl-(C₁-C₈ alkenyl)ₘ-;
Each m is independently an integer of 0 or 1;
Each G and L is independently selected from CH₂, C(O), N(H), N(C₁-C₆ alkyl), O, S, S(O), and S(O)₂;
R⁴ is attached at one of the three substitutable benzo carbon atoms of Formula II and R⁴ and R⁵ are each independently selected from H, CH₃, CF₃, N≡C—, CH₃C(O), HO, CH₃O, C(F)H₂O, C(H)F₂O, CF₃O, F, and Cl.

2. The compound according to claim 1, or a pharmaceutically acceptable salt thereof, wherein G¹ and G² are as defined in claim 1 except each m is 1 and each C₁-C₈ alkylenyl is independently CH₂ or CH₂ substituted with 1 or 2 substituents independently selected from: C₁-C₆ alkyl-(G)ₘ-; C₁-C₆ alkyl; CN; CF₃; HO; (C₁-C₆ alkyl)-O; (C₁-C₆ alkyl)-S; (C₁-C₆ alkyl)-S(O);
(C₁-C₆ alkyl)-S(O)₂; O₂N; H₂N; (C₁-C₆ alkyl)-N(H); (C₁-C₆ alkyl)₂-N; (C₁-C₆ alkyl)-C(O)O—(C₁-C₈ alkylenyl)ₘ-; (C₁-C₆ alkyl)-C(O)O-(1- to 8-membered heteroalkylenyl)ₘ-; (C₁-C₆ alkyl)-C(O)N(H)—(C₁-C₈ alkylenyl)ₘ-; (C₁-C₆ alkyl)-C(O)N(H)-(1- to 8-membered heteroalkylenyl)ₘ-; H₂NS(O)₂—(C₁-C₈ alkylenyl)ₘ-; (C₁-C₆ alkyl)-N(H)S(O)₂—(C₁-C₈ alkylenyl)ₘ-; (C₁-C₆ alkyl)₂-NS(O)₂—(C₁-C₈ alkylenyl)ₘ-; 3- to 6-membered heterocycloalkyl-(G)ₘ-; 5- or 6-membered heteroaryl-(G)ₘ-; (C₁-C₆ alkyl)-S(O)₂—N(H)—C(O)—(C₁-C₈ alkylenyl)ₘ-; (C₁-C₆ alkyl)-C(O)—N(H)—S(O)₂—(C₁-C₈ alkylenyl)ₘ-; Halo; HO₂C; and ═O.

3. The compound according to claim 1, or a pharmaceutically acceptable salt thereof, wherein G¹ and G² are as defined in claim 1 except each m is 1 and each C₁-C₈ alkylenyl is independently CH₂, CHF, CF₂, or C(═O); R⁴ is H or fluoro, and R⁵ is H.

4. The compound according to claim 1, or a pharmaceutically acceptable salt thereof, wherein G¹ is 5- or 6-membered heteroaryl-CH₂, 8- to 10-membered heterobiaryl-CH₂, or a substituted phenyl-CH₂; wherein 5- or 6-membered heteroaryl-CH₂ and 8- to 10-membered heterobiaryl-CH₂ may be independently unsubstituted or substituted as described above for Formula II.

5. The compound according to claim 1, or a pharmaceutically acceptable salt thereof, wherein G² is 5- or 6-membered heteroaryl-CH₂, 8- to 10-membered heterobiaryl-CH₂, or a substituted phenyl-CH₂; wherein 5- or 6-membered heteroaryl-CH₂ and 8- to 10-membered heterobiaryl-CH₂ may be independently unsubstituted or substituted as described above for Formula II.

6. The compound according to claim 1, or a pharmaceutically acceptable salt thereof, wherein:
G¹ and G² each independently are 5- or 6-membered heteroaryl-CH₂, 8- to 10-membered heterobiaryl-CH₂, or a substituted phenyl-CH₂; wherein 5- or 6-membered heteroaryl-CH₂ and 8- to 10-membered heterobiaryl-CH₂ may be independently unsubstituted or substituted as described above for Formula II.

7. The compound according to claim 1, or a pharmaceutically acceptable salt thereof, wherein:
Each G¹ and G² independently are 4-methoxyphenylmethyl,
3-methoxyphenylmethyl, 4-fluorophenylmethyl,
3-fluorophenylmethyl, 4-chlorophenylmethyl,
3-chlorophenylmethyl, 4-bromophenylmethyl,
3-bromophenylmethyl, 4-nitrophenylmethyl, 3-nitrophenylmethyl,
4-methylsulfanylphenylmethyl, 3-methylsulfanylphenylmethyl,
4-methylphenylmethyl, 3-methylphenylmethyl,
4-cyanophenylmethyl, 3-cyanophenylmethyl,
4-carboxyphenylmethyl, 3-carboxyphenylmethyl,
4-methanesulfonylphenylmethyl, 3-methanesulfonylphenylmethyl,
pyridin-4-ylmethyl, pyridin-3-ylmethyl, or pyridin-2-ylmethyl, or
2-methoxypyridin-4-ylmethyl.

8. The compound according to claim 1 selected from:
4-{6-[2-(3-Methoxy-phenyl)-acetylamino]-4-oxo-4H-quinazolin-3-ylmethyl}-benzoic acid;
4-{7-Fluoro-6-[2-(3-methoxy-phenyl)-acetylamino]-4-oxo-4H-quinazolin-3-ylmethyl}-benzoic acid;
4-{6-[2-(4-Methoxy-phenyl)-acetylamino]-4-oxo-4H-quinazolin-3-ylmethyl}-benzoic acid;
4-{6-[2-(4-Fluoro-phenyl)-acetylamino]-4-oxo-4H-quinazolin-3-ylmethyl}-benzoic acid;
4-{6-[2-(3-Fluoro-phenyl)-acetylamino]-4-oxo-4H-quinazolin-3-ylmethyl}-benzoic acid;
4-{7-fluoro-6-[2-(4-methoxyphenyl)acetylamino]-4-oxo-4H-quinazolin-3-ylmethyl}benzoic acid;
4-[7-fluoro-4-oxo-6-(2-p-tolylacetylamino)-4H-quinazolin-3-ylmethyl]benzoic acid; and
4-{7-fluoro-6-[2-(4-hydroxyphenyl)acetylamino]-4-oxo-4H-quinazolin-3-ylmethyl}benzoic acid; or
a pharmaceutically acceptable salt thereof.

9. A pharmaceutical composition, comprising a compound according to claim 1, or a pharmaceutically acceptable salt thereof, admixed with a pharmaceutically acceptable carrier, excipient, or diluent.

10. A method of treating a disease selected from rheumatoid arthritis, osteoarthritis, breast cancer, heart failure, and atherosclerotic plaque rupture in a patient in need thereof, comprising administering to said patient a therapeutically effective amount of a compound according to claim 1, or a pharmaceutically acceptable salt thereof.
